SEQUENCE LISTING (14 sequences)

SEQ ID NO: 1 - DNA, 20 bp, synthetic construct
  misc_feature 1..20: note = Synthetic polynucleotide
  modified_base 1..20: mod_base = OTHER; note = Modified by a phosphorothioate substitution
  misc_feature 20: note = Modified by (SP18)-(SP18)-Cholesterol; SP18 is a hexaethylene glycol spacer 18 molecule
  source 1..20: mol_type = other DNA
tccatgacgttcctgacgtt

SEQ ID NO: 2 - DNA, 12 bp, synthetic construct
  misc_feature 1..12: note = Syntehtic polynucleotide
  source 1..12: mol_type = other DNA
cgacgttcgtcg

SEQ ID NO: 3 - DNA, 13 bp, synthetic construct
  misc_feature 1..13: note = Synthetic polynucleotide
  source 1..13: mol_type = other DNA
cggcgccgtgccg

SEQ ID NO: 4 - DNA, 24 bp, synthetic construct
  misc_feature 1..24: note = Synthetic polynucleotide
  misc_feature 24: note = Modified by (SP18)-(SP18)-TEG-Cholesterol; SP18 is a hexaethylene glycol spacer 18 molecule; TEG is a tetraethyleneglycol linker
  source 1..24: mol_type = other DNA
tcgtcgttttgtcgttttgtcgtt

SEQ ID NO: 5 - DNA, 24 bp, synthetic construct
  misc_feature 1..24: note = Synthetic polynucleotide
  source 1..24: mol_type = other DNA
tcgtcgttttgtcgttttgtcgtt

SEQ ID NO: 6 - DNA, 20 bp, synthetic construct
  misc_feature 1..20: note = Synthetic polynucleotide
  misc_feature 20: note = Modified by (SP18)-(SP18)-TEG-Cholesterol; SP18 is a hexaethylene glycol spacer 18 molecule; TEG is a tetraethyleneglycol linker
  source 1..20: mol_type = other DNA
tccatgacgttcctgacgtt

SEQ ID NO: 7 - DNA, 20 bp, synthetic construct
  misc_feature 1..20: note = Synthetic polynucleotide
  source 1..20: mol_type = other DNA
tccatgacgttcctgacgtt

SEQ ID NO: 8 - DNA, 24 bp, synthetic construct
  misc_feature 1..24: note = Synthetic polynucleotide
  misc_feature 24: note = Modified by (SP18)-(SP18)-TEG-Cholesterol; SP18 is a hexaethylene glycol spacer 18 molecule; TEG is a tetraethyleneglycol linker
  source 1..24: mol_type = other DNA
tgctgcttttgtgcttttgtgctt

SEQ ID NO: 9-14 - skipped (6 entries)

(19) United States
(12) Patent Application Publication
(10) Pub. No.: US 2025/0304974 A1
(43) Pub. Date: Oct. 2, 2025

(54) TLR9-TARGETED SPHERICAL NUCLEIC ACIDS HAVING POTENT ANTITUMOR ACTIVITY

(71) Applicant: Flashpoint Therapeutics, Inc., Palo Alto, CA (US)

(72) Inventors: Subbarao Nallagatla, Chicago, IL (US); Bart Anderson, Chicago, IL (US); Ekambar Kandimalla, Hopkinton, MA (US)

(73) Assignee: Flashpoint Therapeutics, Inc., Palo Alto, CA (US)

(21) Appl. No.: 19/236,381

(22) Filed: Jun. 12, 2025

Related U.S. Application Data
(62) Division of application No. 16/099,409, filed on Nov. 6, 2018, now abandoned.
(60) Provisional application No. 62/333,139, filed on May 6, 2016.
(60) Provisional application No. 62/480,936, filed on Apr. 3, 2017.

(51) Int. Cl. C12N 15/117; A61K 9/00; A61K 31/7125; A61K 39/00; A61K 39/395; A61P 35/00

(52) U.S. Cl. CPC C12N 15/117; A61K 9/0019; A61K 31/7125; A61K 39/3955; A61P 35/00; A61K 2039/54; A61K 2039/545; C12N 2310/17; C12N 2310/315; C12N 2310/51; C12N 2310/532; C12N 2320/31

(57) ABSTRACT

Aspects of the invention relate to immunostimulatory spherical nucleic acids (IS-SNA) for the treatment of a disorder, such as cancer. The IS-SNA may be administered together with a checkpoint inhibitor.

### RELATED APPLICATIONS

This Application is a Divisional of U.S. application Ser. No. 16/099,409, filed Nov. 6, 2018, which is a national stage filing under 35 U.S.C. § 371 of International Patent Application Serial No. PCT/US2017/031423, filed May 5, 2017, which claims the benefit under 35 U.S.C. § 119(e) of U.S. Application Ser. No. 62/480,936, filed Apr. 3, 2017, entitled “TLR-TARGETED SPHERICAL NUCLEIC ACIDS HAVING POTENT ANTITUMOR ACTIVITY” and U.S. Application Ser. No. 62/333,139, filed May 6, 2016, entitled “TLR-TARGETED SPHERICAL NUCLEIC ACIDS HAVING POTENT ANTITUMOR ACTIVITY”. The entire contents of these applications are incorporated herein by reference in their entireties.

### REFERENCE TO AN ELECTRONIC SEQUENCE LISTING

The contents of the electronic sequence listing (F095370011US03-SEQ-DQB.xml; Size: 14,688 bytes; and Date of Creation: Jun. 12, 2025) is herein incorporated by reference in its entirety.

### BACKGROUND OF INVENTION

The immune system is a highly evolved, exquisitely precise endogenous mechanism for clearing foreign, harmful, and unnecessary material including pathogens and senescent or malignant host cells. It is known that modulating the immune system for therapeutic or prophylactic purposes is possible by introducing compounds that modulate the activity of specific immune cells. Among the immunostimulatory compounds being developed, agonists of Toll-like receptors (TLR) have demonstrated outstanding potential. Agonists of TLR4, such as monophosphoryl lipid A (MPL) have reached late stages of clinical trials and approval in various countries in some instances. Despite these promising results, there is still a clear and significant need for compounds which can safely and effective induce responses that can clear intracellular pathogens and cancers, such as inducers of cell-mediated immunity. Agonists of TLR 3, TLR 7/8 and TLR 9 have excellent potential due to their potent ability to induce Th1 cell-mediated immune responses. A synthetic TLR 7/8 agonist, imiquimod, has been approved to treat various skin diseases, including superficial carcinomas and genital warts, and is being developed for a variety of other indications. Similarly, agonists of TLR 9 are in various stages of clinical development, for treatment of various diseases with large unmet medical needs. However, concerns due to lack of efficacy, off-target phosphorothioate effects, and toxicity have slowed effective clinical translation of TLR 7/8 and 9 agonists.

### SUMMARY OF INVENTION

Some aspects of the present disclosure include an immunostimulatory spherical nucleic acid (IS-SNA), comprising a core having an oligonucleotide shell comprised of immunostimulatory oligonucleotides positioned on the exterior of the core and a checkpoint inhibitor.

In some embodiments, the core is a solid or hollow core. In another embodiment, the core is a solid core comprised of noble metals, including gold and silver, transition metals including iron and cobalt, metal oxides including silica, polymers or combinations thereof. In other embodiments, the core is a solid polymeric core and wherein the polymeric core is comprised of amphiphilic block copolymers, hydrophobic polymers including polystyrene, poly(lactic acid), poly(lactic co-glycolic acid), poly(glycolic acid), poly(caprolactone) and other biocompatible polymers.

In some embodiments, the core is a liposomal core. In another embodiment, the liposomal core is comprised of one or more lipids selected from: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof. In other embodiments, the liposomal core is comprised of one type of lipid. In another embodiment, the liposomal core is comprised of 2-10 different lipids.

In some embodiments, the checkpoint inhibitor is incorporated into the liposomal core. In another embodiment, the checkpoint inhibitor is coformulated in a composition with the IS-SNA. In other embodiments, the checkpoint inhibitor is selected from the group consisting of a monoclonal antibody, a humanized antibody, a fully human antibody, a fusion protein or a combination thereof or a small molecule. In another embodiment, the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. The checkpoint inhibitor, in some embodiments, is an anti-PD-1 antibody. In some embodiments, the anti-PD-1 antibody is BMS-936558 (nivolumab). In some embodiments, the checkpoint inhibitor is an anti-PDL1 antibody. In another embodiment, the anti-PDL1 antibody is MPDL3280A (atezolizumab). In another embodiment, the checkpoint inhibitor is an anti-CTLA-4 antibody. In other embodiments, the anti-CTLA-4 antibody is ipilimumab.

In some embodiments, one or more of the immunostimulartory oligonucleotides comprises a sequence selected from the group consisting of SEQ ID NO: 4, SEQ ID NO: 5, SEQ ID NO:6 and SEQ ID NO: 7.

Some aspects of the disclosure include a method for treating cancer, including administering by intravenous injection to a subject having cancer an immunostimulatory spherical nucleic acid (IS-SNA), comprising a core and an oligonucleotide shell comprised of immunostimulatory oligonucleotides positioned on the exterior of the core in an effective amount to treat the cancer.

In some embodiments, the IS-SNA is administered to the subject at least 4 times, each administration separated by at least 3 days. In other embodiments, the IS-SNA is administered to the subject weekly for 4-12 weeks.

In some embodiments, the method further includes administering to the subject a checkpoint inhibitor. In other embodiments, the IS-SNA and check point inhibitor are administered on the same days. In another embodiment, the IS-SNA and checkpoint inhibitor are administered on different days. In some embodiments, the checkpoint inhibitor is administered before the IS-SNA.

In some embodiments, the IS-SNA induces cytokine secretion. In some embodiments, the IS-SNA induces TH1-type cytokine secretion. In certain embodiments, the immunostimulatory oligonucleotide in the IS-SNA increases the ratio of T-effector cells to T-regulatory cells relative to a linear immunostimulatory oligonucleotide not linked to an IS-SNA.

In some embodiments, the IS-SNA is any of the IS-SNA described herein. In some embodiments, the IS-SNA targets a TLR9 receptor in a cell in the subject.

In some embodiments, the subject is a mammal. In certain embodiments, the subject is human.

In some embodiments, the cancer is selected from the group consisting of biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non small cell); melanoma; neuroblastomas; oral cancer; ovarian cancer; pancreas cancer; prostate cancer; rectal cancer; sarcomas; skin cancer; testicular cancer; thyroid cancer; and renal cancer.

Other aspects of the disclosure provide a method for treating cancer, including administering to a subject having cancer in an effective amount to treat the cancer an immunostimulatory spherical nucleic acid (IS-SNA), comprising a core and an oligonucleotide shell comprised of immunostimulatory oligonucleotides positioned on the exterior of the core and a checkpoint inhibitor.

In some embodiments, the combined administration of IS-SNA and checkpoint inhibitor produces a synergistic effect on survival of the subject.

In other embodiments, the IS-SNA and checkpoint inhibitor are administered on the same days. In another embodiment, the IS-SNA and checkpoint inhibitor are administered on different days. In other embodiments, the checkpoint inhibitor is administered before the IS-SNA.

In some embodiments, the IS-SNA induces cytokine secretion. In some embodiments, the IS-SNA induces TH1-type cytokine secretion. In certain embodiments, the immunostimulatory oligonucleotide in the IS-SNA increases the ratio of T-effector cells to T-regulatory cells relative to a linear immunostimulatory oligonucleotide not linked to an IS-SNA.

In some embodiments, the IS-SNA is any of the IS-SNA described herein. In some embodiments, the IS-SNA targets a TLR9 receptor in a cell in the subject.

In some embodiments, the subject is a mammal. In certain embodiments, the subject is human.

In some embodiments, the checkpoint inhibitor is selected from the group consisting of a monoclonal antibody, a humanized antibody, a fully human antibody, a fusion protein or a combination thereof or a small molecule. In another embodiment, the checkpoint inhibitor inhibits a checkpoint protein selected from the group consisting of CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an anti-PD-1 antibody. In another embodiment, the anti-PD-1 antibody is BMS-936558 (nivolumab). In some embodiments, the checkpoint inhibitor is an anti-PDL1 antibody. In another embodiment, the anti-PDL1 antibody is MPDL3280A (atezolizumab). In other embodiments, the checkpoint inhibitor is an anti-CTLA-4 antibody. In some embodiments, the anti-CTLA-4 antibody is ipilimumab.

In some embodiments, the IS-SNA induces cytokine secretion. In some embodiments, the IS-SNA induces TH1-type cytokine secretion. In certain embodiments, the immunostimulatory oligonucleotide in the IS-SNA increases the ratio of T-effector cells to T-regulatory cells relative to a linear immunostimulatory oligonucleotide not linked to an IS-SNA.

In some embodiments, the IS-SNA is any of the IS-SNA described herein. In some embodiments, the IS-SNA targets a TLR9 receptor in a cell in the subject.

In some embodiments, the subject is a mammal. In certain embodiments, the subject is human.

The present disclosure, in other aspects, provides a method for treating cancer, including administering by intratumoral or subcutaneous injection to a subject having cancer an immunostimulatory spherical nucleic acid (IS-SNA), comprising a core and an oligonucleotide shell comprised of immunostimulatory oligonucleotides positioned on the exterior of the core in an effective amount to treat the cancer, wherein the IS-SNA is administered to the subject at least 4 times, each administration separated by at least 3 days.

In some embodiments, the core is a solid or hollow core. In other embodiments, the core is a solid core comprised of noble metals, including gold and silver, transition metals including iron and cobalt, metal oxides including silica, polymers or combinations thereof. In another embodiment, the core is a solid polymeric core and wherein the polymeric core is comprised of amphiphilic block copolymers, hydrophobic polymers including polystyrene, poly(lactic acid), poly(lactic co-glycolic acid), poly(glycolic acid), poly(caprolactone) and other biocompatible polymers.

In some embodiments, the core is a liposomal core. In other embodiments, the liposomal core is comprised of one or more lipids selected from: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and derivatives thereof. In some embodiments, the liposomal core is comprised of one type of lipid. In other embodiments, the liposomal core is comprised of 2-10 different lipids.

In some embodiments, the immunostimulatory oligonucleotides are CpG oligonucleotides. In other embodiments, the CpG oligonucleotides are B-class CpG oligonucleotides. In another embodiment, the CpG oligonucleotides are C-class CpG oligonucleotides. In some embodiments, the CpG oligonucleotides are A-class CpG oligonucleotides. In other embodiments, the CpG oligonucleotides are a mixture of A-class CpG oligonucleotides, B-class CpG oligonucleotides and C-class CpG oligonucleotides. In a further embodiment, the CpG oligonucleotides are 4-100 nucleotides in length.

In some embodiments, the oligonucleotides of the oligonucleotide shell are oriented radially outwards. In other embodiments, the oligonucleotide shell has a density of 5-1,000 oligonucleotides per SNA. In another embodiment, the oligonucleotide shell has a density of 100-1,000 oligonucleotides per SNA. In still another embodiment, the oligonucleotide shell has a density of 500-1,000 oligonucleotides per SNA.

In some embodiments, the oligonucleotides have at least one internucleoside phosphorothioate linkage. In other embodiments, each of the internucleoside linkages of the CpG oligonucleotides are phosphorothioate.

In some embodiments, the IS-SNA induces cytokine secretion. In some embodiments, the IS-SNA induces TH1-type cytokine secretion. In certain embodiments, the immunostimulatory oligonucleotide in the IS-SNA increases the ratio of T-effector cells to T-regulatory cells relative to a linear immunostimulatory oligonucleotide not linked to an IS-SNA.

In some embodiments, the IS-SNA is any of the IS-SNA described herein. In some embodiments, the IS-SNA targets a TLR9 receptor in a cell in the subject.

In some embodiments, the subject is a mammal. In certain embodiments, the subject is human.

The present disclosure, in other aspects, provides a method for treating a disorder, including nasally or intramuscularly administering to a subject having the disorder in an effective amount to treat the disorder an immunostimulatory spherical nucleic acid (IS-SNA), including a core and an oligonucleotide shell comprised of immunostimulatory oligonucleotides positioned on the exterior of the core and a checkpoint inhibitor. In certain embodiments, the disorder is cancer.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is a schematic diagram of the study design for subcutaneous and intratumoral delivery of IS-SNA (3.2 and 6.4 mg/kg) in CT26 tumor-containing Balb/c mice.

FIG. 2 shows the resulting tumor growth and survival (mean±SD, N=8 per group) after subcutaneous delivery of IS-SNA (3.2 and 6.4 mg/kg) in CT26 tumor-containing Balb/c mice.

FIG. 3 shows the resulting tumor growth and survival (mean±SD, N=8 per group) after intratumoral delivery of IS-SNA (3.2 and 6.4 mg/kg) in CT26 tumor-containing Balb/c mice.

FIG. 4 is a schematic diagram of the study design for intratumoral delivery of IS-SNA (0.8, 3.2 and 6.4 mg/kg) in MC38 tumor-containing C57bl/6 mice.

FIG. 5 shows the resulting tumor growth curves (mean±SD, N=10 per group) after intratumoral delivery of IS-SNA (0.8, 3.2 and 6.4 mg/kg) in MC38 tumor-containing C57bl/6 mice.

FIG. 6 shows the resulting survival curves (mean±SD, N=10 per group) after intratumoral delivery of IS-SNA (0.8, 3.2 and 6.4 mg/kg) in MC38 tumor-containing C57bl/6 mice.

FIG. 7 is a schematic diagram of the study design for intravenous delivery of IS-SNA (0.8 mg/kg) in EMT-6 tumor-containing Balb/c mice.

FIG. 8 shows the resulting tumor growth curves (mean±SD, N=8 per group) after intravenous delivery of IS-SNA (0.8 mg/kg) in EMT-6 tumor-containing Balb/c mice.

FIG. 9 shows the resulting survival curves (mean±SD, N=8 per group) after intravenous delivery of IS-SNA (0.8 mg/kg) in EMT-6 tumor-containing Balb/c mice.

FIG. 10 is a schematic diagram of the study design for the subcutaneous delivery of IS-SNA (0.8 mg/kg) in EMT-6 tumor-bearing Balb/c mice.

FIG. 11 shows the resulting tumor growth curves (mean±SD, N=8 per group) after subcutaneous delivery of IS-SNA (0.8 mg/kg) in EMT-6 tumor-bearing Balb/c mice.

FIG. 12 shows the ratios of effector to regulatory T cells in the draining lymph nodes of EMT-6 tumor-bearing Balb/c mice (mean±SD, N=8 per group) following the subcutaneous delivery of IS-SNA (0.8 mg/kg).

FIG. 13 is a schematic diagram of the study design for the subcutaneous delivery of IS-SNA (0.8 mg/kg) in B16F10 melanoma-containing C57bl/6 mice.

FIG. 14 shows the resulting tumor growth curves (mean±SD, N=10 per group) after the subcutaneous delivery of IS-SNA (0.8 mg/kg) in B16F10 melanoma-containing C57bl/6 mice.

FIGS. 15A-15C show uptake and TLR9 activation by TLR9 agonist SNAs. In FIG. 15A, human PBMCs were treated with fluorescein-labeled SNA1 or linear oligo 2 TLR9 agonist oligonucleotides. After 24 hours, the fraction of cells with cell-associated fluorescein-labeled compound was assessed by flow cytometry. FIG. 15B shows activation of human TLR9 in reporter cells by TLR9 agonists. hTLR9-HEK-Blue reporter cells were treated with SNA1, Linear oligo 2, or Control SNA5 (containing GpC in place of CpG) for 4 hours. The media was replaced and cells were incubated an additional 20 hours. NF-κB activation was assessed using the QUANTI-Blue reporter assay. Mean±SEM of three independent experiments are shown. P-values: *<0.05, **<0.005, ****<0.0001. FIG. 15C shows specificity of TLR9 agonist SNAs. HEK-Blue reporter cells overexpressing no TLR (null1), hTLR3, hTLR7, hTLR8, or hTLR9 were treated with 5 μM SNA1 or 85 nM poly I:C (hTLR3), 0.5 μM SNA1 or 1 μM R848 (hTLR7, hTLR8), 5 μM SNA1 or 5 μM Control SNA5 (hTLR9), and 5 μM SNA1 or 10 μg/mL PMA (null1) for 24 hours. NF-κB activation was assessed as described in FIG. 17B legend. Mean+SEM of n=3 or 4 independent repetitions is displayed. ***P<0.001, ****P<0.0001.

FIG. 16 shows uptake of TLR9 agonist oligonucleotides in SNA and linear formats by human PBMC. Human PBMC were treated with fluorescein-labeled SNA1 or linear oligo 2. After 24 hours, flow cytometry was used to assess the amount of cell-associated oligos per cell. Mean+SEM, n=4 donors. P-values: **<0.01, ****<0.0001.

FIGS. 17A-17D show cytokine induction in primary leukocytes and in vivo in mice by TLR9 agonist SNAs compared with linear oligonucleotides. Multiplex ELISAs were used to quantify cytokines in the cell culture supernatant of primary leukocytes treated for 24 hours with TLR9 agonists (FIGS. 17A and 17B) or in mouse serum following subcutaneous administration of TLR9 agonists (FIGS. 17C and 17D); mean+SEM of four mice is shown. FIG. 17A shows mouse splenocytes treated with SNA3, Linear oligo 4, or PBS. Cells were treated with 10 μM oligonucleotide, or 1 μM oligonucleotide for IFN-γ. Mean+SD of duplicate wells is displayed and is representative of n=3 independent experiments. FIG. 17B shows human PBMC treated with 2.5 μM SNA1, linear oligo 2, control SNA5, or PBS. Mean and individual responses of 7-13 independent donors are shown. Paired T-test p-values*<0.05, **<0.01. FIG. 17C shows the time course of serum cytokine response at 3 mg/kg SNA3 in mice. FIG. 17D shows dose-dependent serum cytokine response to SNA3 in mice.

FIGS. 18A-18B show cytokine induction in primary leukocytes by TLR9 agonist SNAs. Multiplex ELISAs were used to quantify cytokines in the cell culture supernatant of primary leukocytes treated for 24 hours with TLR9 agonists. FIG. 18A shows TH2 and TH17 cytokine induction in mouse splenocytes treated with SNA3, Linear oligo 4, or PBS. Cells were treated with 10 μM oligonucleotide. Mean+SD of duplicate wells is displayed and is representative of n=3 independent experiments. FIG. 18B shows dose response of cytokine induction in primary hPBMC by SNA1 and Control SNA5. Mean+SEM of duplicate wells from one donor is shown and is representative of seven independent experiments (donors).

FIGS. 19A-19B show in vivo murine serum cytokine responses to TLR9 agonist SNAs. Multiplex ELISAs were used to quantify cytokines in murine serum following subcutaneous administration. Mean+SEM of four mice is shown. FIG. 19A shows time course following administration of 7.5 mg/kg of SNA1. FIG. 19B shows dose-response to SNA1 and Control SNA5.

FIGS. 20A-20C show in vivo response to subcutaneously administered SNA1 and control SNA5 in non-human primates. Cynomolgus monkeys were administered with SNA1 or control SNA5 at indicated doses. Mean+SEM of n=4 monkeys is displayed. FIG. 20A shows immune cell activation as measured by flow cytometry of PBMCs 24 hr post dosing. FIG. 20B shows serum cytokine levels at 12 hr post dosing. FIG. 20C shows the time course of serum cytokine induction at 1 mg/kg dose.

FIG. 21 shows in vivo hematological changes to subcutaneously administered SNA1 in non-human primates. Cynomolgus monkeys were injected subcutaneously with SNA1 at indicated doses. Mean+SEM of n=4 monkeys is displayed.

FIGS. 22A-22F show SNA monotherapy and combination with anti-PD-1 in mice bearing MC38 tumors. Mice were inoculated subcutaneously with MC38 colorectal cells to establish flank tumors. Dosing of SNA and anti-PD-1 began after tumors reached 100 mm3 and occurred every three days for a total of five doses (indicated by arrows). SNAs were injected intratumorally at the indicated dose level. Anti-PD-1 was administered intraperitoneally at 5 mg/kg. Mean tumor volume+SEM of n-8 mice is displayed. **** P<0.0001 vs. vehicle on day 23. Tumor growth inhibition (TGI) compared to vehicle on day 23. FIG. 22A shows SNA3 monotherapy. FIG. 22B shows SNA3 combination with anti-PD-1. FIGS. 22C and 22D show SNA3 monotherapy and combination therapy with once or twice weekly dosing. Once weekly dosing indicated by hooks. FIG. 22E shows SNA1 or SNA3 monotherapy. FIG. 22F shows survival of mice previously treated with SNA3 (1.6 mg/kg twice weekly) in combination with anti-PD-1 following intraperitoneal (IP) challenge with MC38 colorectal cells. SNA3+anti-PD-1 n=4 mice, naïve mice n=6.

FIG. 23 shows cytokine response to SNA3 administration in mice bearing MC38 tumors. Four hours following the first (day 9) dose of SNA3, serum cytokine responses were assessed in mice bearing MC38 tumors. Mean and individual responses of n=4 mice are displayed. P-values: *<0.05, **<0.01, ***<0.001, ****<0.0001.

FIGS. 24A-24F show EMT6 tumors treated with SNA as monotherapy and in combination with anti-PD-1. In mice bearing EMT6 flank tumors, beginning at 100 mm³ Mverage tumor volume (MTV) (FIG. 24A-24C) or three days after tumor inoculation (d3) (FIG. 24D), SNA3, SNA1, control SNA5, or linear oligo 4 was injected subcutaneously every three days (FIG. 24A, 24B, 24D) or weekly (FIG. 24C) (5 total doses indicated by arrows). FIG. 24A shows SNA3 monotherapy. MTV+SEM, n=8 mice. *P<0.05, ****P<0.0001 vs vehicle d27. FIG. 24B shows SNA3 monotherapy in mice bearing tumors on both flanks. MTV+SEM, n=16. *P<0.05, ****P<0.0001 vs vehicle d34. FIG. 24C shows SNA1 or control SNA5 monotherapy. MTV+SEM, n=10. ****P<0.0001 vs vehicle d25. FIG. 24D shows SNA3+anti-PD-1 combination. Beginning d3, SNA3 or Linear oligo 4 injected subcutaneously and 10 mg/kg anti-PD-1 injected intraperitoneally every 5 days (3 doses; open arrows). MTV+SEM, n=8. TGI vs vehicle d27. FIG. 24E shows mice subsequently rechallenged on opposite flank with the same tumor cell line (EMT6). MTV+SEM, n=7. FIG. 24F shows mice subsequently challenged with distinct tumor cell lines from the same tissue (4T1 breast) or a dissimilar tissue (CT26 colorectal). MTV+SEM, n=3 each.

FIGS. 25A-25D show biomarkers of SNA-induced anti-tumor immunity in mice bearing EMT6 tumors. Mice were inoculated subcutaneously with EMT6 breast tumor cells to establish flank tumors. Beginning three days after tumor inoculation, SNA3 or Linear oligo 4 was injected subcutaneously every three days and anti-PD-1 was injected every 5 days. FIG. 25A shows tumor growth. Mean tumor volume+SEM is displayed. P-value and TGI are compared to PBS on day 27. ****p<0.0001. FIGS. 25B-25D: From five mice on day 10 following tumor inoculation, FIG. 25B: the tumors were removed for examination by immunohistochemistry, FIG. 25C: the draining lymph nodes were removed for flow cytometry assessment, and FIG. 25D: the tumors were examined for mMDSC by flow cytometry assessment. P values: *<0.05, **<0.01, ***<0.001 vs PBS; #<0.05, ##<0.01, ###<0.001 vs anti-PD-1; \hat{ }\hat{ }\hat{ }<0.001 vs SNA3.

FIG. 26 shows serum cytokine response in mice to intravenously administered SNA. Multiplex ELISAs were used to quantify cytokines in murine serum following subcutaneous (s.c.) or intravenous (i.v.) administration of 7.5 mg/kg SNA1. Mean and individual responses of n=4 mice is displayed. P-values vs PBS: *<0.05, **<0.01, ***<0.001, ****<0.0001.

FIGS. 27A-27C show intravenous administration of SNA in mice bearing EMT6 tumors. Mice were inoculated subcutaneously with EMT6 breast tumor cells to establish flank tumors. Three days after tumor inoculation, SNA3 was injected intravenously at the indicated dose level every three days for a total of five doses (dosing events indicated by arrows) as a monotherapy (FIG. 27A) and in combination with anti-PD-1 antibody (FIG. 27B). Mean+SEM of n=8 mice is displayed. P-values vs vehicle on day 20: **<0.01, ***<0.001, ****<0.0001. TGI compared to vehicle on day 20. FIG. 27C shows EMT6 tumor rechallenge in mice treated with intravenous administration of SNA combination therapy. On day 65, the surviving mice in SNA+anti-PD-1 combination therapy groups were subcutaneously rechallenged with 1× (1 million) or 2× (2 million) EMT6 cells on the contralateral flank. Mean+SEM of n=6 mice is displayed. P-values vs naïve mice on day 95: ****<0.0001.

### DETAILED DESCRIPTION

The use of Immunostimulatory Spherical Nucleic Acid, referred herein as IS-SNA, for treating cancer as a monotherapy and/or in combination with checkpoint inhibitors and other therapeutics is described herein. IS-SNAs are a novel class of agent that consists of immunostimulatory oligonucleotides densely packed and radially oriented around a spherical lipid bilayer. These structures exhibit the ability to enter cells without the need for auxiliary delivery vehicles or transfection reagents, by engaging scavenger receptors and lipid rafts.

It was discovered, surprisingly, according to the invention that IS-SNA are capable of effectively delivering immunostimulatory oligonucleotides to a tumor when administered by an intravenous route. Prior studies of linear TLR9 targeting immunostimulatory oligonucleotides did not produce therapeutic immune responses in healthy human volunteers in a clinical trial (1). Thus, it was quite surprising when it was discovered herein that not only can immunostimulatory oligonucleotides be delivered to a subject by an intravenous route and produce an immune response, but such intravenously administered oligonucleotides showed potent antitumor activity. As shown in the Examples, set forth herein, intravenous administration of IS-SNA in an EMT-6 tumor model showed significant reductions in tumor volume compared to a negative control. These findings demonstrate the feasibility of intravenous delivery of IS SNA for the treatment of cancer.

The antitumor effects of IS-SNA as a monotherapy in various syngeneic mouse tumor models, such as CT26 colorectal cancer, MC38 colon cancer, EMT-6 breast cancer and B16F10 melanoma, and as combination therapy with a-PD-1 in EMT-6 and B16F10 models, have been investigated. Several routes of administration (subcutaneous, intratumoral and intravenous) of IS-SNA have been used herein in tumor models for assessing whether different routes of administration are amenable in treating cancer patients. Interestingly, subcutaneous and intratumoral delivery of IS-SNA in an in vivo tumor model showed similar robust antitumor activity, suggesting that both routes of administration of IS-SNA are desirable. In addition, intratumoral delivery of IS-SNA at 6.4 mg/kg dose in an MC38 tumor model led to tumor regression.

It has also been discovered herein that the combination of IS-SNA and checkpoint inhibitors results in a synergistic therapeutic response when administered in vivo. Checkpoint inhibitors such as PD-1 have been shown to play a role in immune regulation and the maintenance of peripheral tolerance (2). Interactions of PD-L1 expressed on tumor cells with PD-1 on T-cells have been shown to attenuate T-cell activation, thereby impairing the antitumor activity of T cells on tumors. Several monoclonal antibodies that inhibit PD-1 and PD-L1 interaction have demonstrated antitumor activity in many tumors. However, the response rate is lower in certain tumor types-for example, only 18% response rate in triple negative breast cancer patients (3). The combined therapy of the invention will provide immense benefit to cancer patients by improving the efficacy of checkpoint inhibitor therapy. In particular it was demonstrated herein that the combination of IS-SNA and checkpoint inhibitors (i.e. PD1 inhibitors) in two animal models that are resistant to a-PD-1 activity (EMT-6 breast cancer and B16F10 melanoma mouse tumor models) produced potent anti-tumor responses. The results shown in the examples demonstrate that IS-SNA in combination with PD-1 inhibitor provide more potent antitumor effects than IS-SNA alone in both of these models. The results were synergistic in both a decrease in tumor volume and an increase in survival time. Together these studies demonstrate the utility of IS-SNAs as immuno-oncology agents in combination with checkpoint inhibitors.

Thus, in some aspects the invention relates to a combination therapy of IS-SNA and checkpoint inhibitors. The IS-SNA may be administered in conjunction with a checkpoint inhibitor. The term “in conjunction with” or “co-administered” refers to a therapy which involves the delivery of the two therapeutics to a patient or subject. The two therapies may be delivered together in a single composition, at the same time, in separate compositions using the same or different routes of administration, or at different times using the same or different routes of administration.

In some embodiments, the IS-SNA and the checkpoint inhibitor are both administered to a subject. The timing of administration of both may vary. In some embodiments, it is preferred that the checkpoint inhibitor be administered subsequent to the administration of the IS-SNA. In some embodiments, the IS-SNA is administered to the subject prior to as well as either substantially simultaneously with or following the administration of the checkpoint inhibitor. The administration of the IS-SNA and the checkpoint inhibitor may also be mutually exclusive of each other so that at any given time during the treatment period, only one of these agents is active in the subject. Alternatively, and preferably in some instances, the administration of the two agents overlaps such that both agents are active in the subject at the same time.

In some embodiments, the IS-SNA is administered on a weekly or biweekly basis and the checkpoint inhibitor is administered more frequently (e.g., on a daily basis). However, if the dose of IS-SNA is reduced sufficiently, it is possible that the IS-SNA is administered as frequently as the checkpoint inhibitor, albeit at a reduced dose.

In some instances, the IS-SNA and/or the checkpoint inhibitor are administered substantially prior to or following a surgery to remove a tumor. As used herein, “substantially prior to or following” means at least six months, at least five months, at least four months, at least three months, at least two months, at least one month, at least three weeks, at least two weeks, at least one week, at least 5 days, or at least 2 days prior to or following the surgery to remove a tumor.

Similarly, the IS-SNA may be administered immediately prior to or following the administration of the checkpoint inhibitor (e.g., within 48 hours, within 24 hours, within 12 hours, within 6 hours, within 4 hours, within 3 hours, within 2 hours, within 1 hour, within 30 minutes or within 10 minutes of the administration), or substantially simultaneously with the checkpoint inhibitor (e.g., during the time the subject is receiving the checkpoint inhibitor).

In other embodiments of the invention, the IS-SNA is administered on a routine schedule. The checkpoint inhibitor may also be administered on a routine schedule, but alternatively, may be administered as needed. A “routine schedule” as used herein, refers to a predetermined designated period of time. The routine schedule may encompass periods of time which are identical or which differ in length, as long as the schedule is predetermined. For instance, the routine schedule may involve administration of the IS-SNA on a daily basis, every two days, every three days, every four days, every five days, every six days, a weekly basis, a bi-weekly basis, a monthly basis, a bi-monthly basis or any set number of days or weeks there-between, every two months, three months, four months, five months, six months, seven months, eight months, nine months, ten months, eleven months, twelve months, etc. Alternatively, the predetermined routine schedule may involve administration of the IS-SNA on a daily basis for the first week, followed by a monthly basis for several months, and then every three months after that. Any particular combination would be covered by the routine schedule as long as it is determined ahead of time that the appropriate schedule involves administration on a certain day.

Checkpoint proteins include but are not limited to PD-1, TIM-3, VISTA, A2AR, B7-H3, B7-H4, BTLA, CTLA-4, IDO, KIR and LAG3. CTLA-4, PD-1 and its ligands are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows them to gain control of the PD-1 pathway and switch off T cells expressing PD-1 that may enter the tumor microenvironment, thus suppressing the anticancer immune response. Pembrolizumab (formerly MK-3475 and lambrolizumab, trade name Keytruda) is a human antibody used in cancer immunotherapy. It targets the PD-1 receptor.

IDO, Indoleamine 2,3-dioxygenase, is a tryptophan catabolic enzyme, which suppresses T and NK cells, generates and activates Tregs and myeloid-derived suppressor cells, and promotes tumor angiogenesis. TIM-3, T-cell Immunoglobulin domain and Mucin domain 3, acts as a negative regulator of Th1/Tc1 function by triggering cell death upon interaction with its ligand, galectin-9. VISTA, V-domain Ig suppressor of T cell activation.

The checkpoint inhibitor may be a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101).

The IS-SNA is comprised of densely packed, radially oriented nucleic acids which stimulate an immune response, and in particular stimulate the toll-like receptors (TLR) such as TLR9. In some embodiments the IS-SNA is an agonist of a TLR (TLR agonist). A TLR agonist, as used herein is a nucleic acid molecule that interacts with and stimulates the activity of a TLR. The IS-SNA, in some embodiments, is a TLR-9 targeted Immunostimulatory Sperical Nucleic Acid.

Toll-like receptors (TLRs) are a family of highly conserved polypeptides that play a critical role in innate immunity in mammals. At least ten family members, designated TLR1-TLR10, have been identified. The cytoplasmic domains of the various TLRs are characterized by a Toll-interleukin 1 (IL-1) receptor (TIR) domain. Medzhitov R et al. (1998) Mol Cell 2:253-8. Recognition of microbial invasion by TLRs triggers activation of a signaling cascade that is evolutionarily conserved in Drosophila and mammals. The TIR domain-containing adaptor protein MyD88 has been reported to associate with TLRs and to recruit IL-1 receptor-associated kinase (IRAK) and tumor necrosis factor (TNF) receptor-associated factor 6 (TRAF6) to the TLRs. The MyD88-dependent signaling pathway is believed to lead to activation of NF-κB transcription factors and c-Jun NH2 terminal kinase (Jnk) mitogen-activated protein kinases (MAPKs), critical steps in immune activation and production of inflammatory cytokines. For a review, see Aderem A et al. (2000) Nature 406:782-87.

TLRs are believed to be differentially expressed in various tissues and on various types of immune cells. For example, human TLR7 has been reported to be expressed in placenta, lung, spleen, lymph nodes, tonsil and on plasmacytoid precursor dendritic cells (pDCs). Chuang T-H et al. (2000) Eur Cytokine Netw 11:372-8); Kadowaki N et al. (2001) J Exp Med 194:863-9. Human TLR8 has been reported to be expressed in lung, peripheral blood leukocytes (PBL), placenta, spleen, lymph nodes, and on monocytes. Kadowaki N et al. (2001) J Exp Med 194:863-9; Chuang T-H et al. (2000) Eur Cytokine Netw 11:372-8. Human TLR9 is reportedly expressed in spleen, lymph nodes, bone marrow, PBL, and on pDCs, and B cells. Kadowaki N et al. (2001) J Exp Med 194:863-9; Bauer S et al. (2001) Proc Natl Acad Sci USA 98:9237-42; Chuang T-H et al. (2000) Eur Cytokine Netw 11:372-8.

Nucleotide and amino acid sequences of human and murine TLR9 are known. See, for example, GenBank Accession Nos. NM_017442, AF259262, AB045180, AF245704, AB045181, AF348140, AF314224, NM_031178; and NP_059138, AAF72189, BAB19259, AAF78037, BAB19260, AAK29625, AAK28488, and NP_112455, the contents of all of which are incorporated herein by reference. Human TLR9 is reported to exist in at least two isoforms, one 1032 amino acids long and the other 1055 amino acids. Murine TLR9 is 1032 amino acids long. TLR9 polypeptides include an extracellular domain having a leucine-rich repeat region, a transmembrane domain, and an intracellular domain that includes a TIR domain.

As used herein, the term “TLR9 signaling” refers to any aspect of intracellular signaling associated with signaling through a TLR9. As used herein, the term “TLR9-mediated immune response” refers to the immune response that is associated with TLR9 signaling. A TLR9-mediated immune response is a response associated with TLR9 signaling. This response is further characterized at least by the production/secretion of IFN-γ and IL-12, albeit at levels lower than are achieved via a TLR8-mediated immune response.

The term “TLR9 agonist” refers to any agent that is capable of increasing TLR9 signaling (i.e., an agonist of TLR9). TLR9 agonists specifically include, without limitation, immunostimulatory oligonucleotides, and in particular CpG immunostimulatory oligonucleotides.

An “immunostimulatory oligonucleotide” as used herein is any nucleic acid (DNA or RNA) containing an immunostimulatory motif or backbone that is capable of inducing an immune response. An induction of an immune response refers to any increase in number or activity of an immune cell, or an increase in expression or absolute levels of an immune factor, such as a cytokine. Immune cells include, but are not limited to, NK cells, CD4+ T lymphocytes, CD8+ T lymphocytes, B cells, dendritic cells, macrophage and other antigen-presenting cells.

As used herein, the term “CpG oligonucleotides,” “immunostimulatory CpG nucleic acids” or “immunostimulatory CpG oligonucleotides” refers to any CpG-containing oligonucleotide that is capable of activating an immune cell. At least the C of the CpG dinucleotide is typically unmethylated. Immunostimulatory CpG oligonucleotides are described in a number of issued patents and published patent applications, including U.S. Pat. Nos. 6,194,388; 6,207,646; 6,218,371; 6,239,116; 6,339,068; 6,406,705; and 6,429,199.

In some embodiments, the CpG oligonucleotides are 4-100 nucleotides in length. In other embodiments, the CpG oligonucleotides are 4-90, 4-80, 4-70, 4-60, 4-50, 4-40, 4-30, 4-20, or 4-10 nucleotides in length.

In some embodiments the immunostimulatory oligonucleotides have a modified backbone such as a phosphorothioate (PS) backbone. In other embodiments the immunostimulatory oligonucleotides have a phosphodiester (PO) backbone. In yet other embodiments immunostimulatory oligonucleotides have a mixed PO and PS backbone. The CpG oligonucleotides may be A-class oligonucleotides, B-class oligonucleotides, or C-class oligonucleotides. “A-class” CpG immunostimulatory oligonucleotides have been described in published PCT application WO 01/22990. These oligonucleotides are characterized by the ability to induce high levels of interferon-alpha while having minimal effects on B cell activation. The A class CpG immunostimulatory nucleic acid may contain a hexamer palindrome GACGTC, AGCGCT, or AACGTT described by Yamamoto and colleagues. Yamamoto S et al. J Immunol 148:4072-6 (1992). Traditional A-class oligonucleotides have poly-G rich 5′ and 3′ ends and a palindromic center region. Typically the nucleotides at the 5′ and 3′ ends have stabilized internucleotide linkages and the center palindromic region has phosphodiester linkages (chimeric).

B class CpG immunostimulatory nucleic acids strongly activate human B cells but have minimal effects inducing interferon-α without further modification. Traditionally, the B-class oligonucleotides include the sequence 5′ TCN₁TX₁X₂CGX₃X₄ 3′, wherein X₁ is G or A; X₂ is T, G, or A; X₃ is T or C and X₄ is T or C; and N is any nucleotide, and N₁ and N₂ are nucleic acid sequences of about 0-25 N's each. B-class CpG oligonucleotides that are typically fully stabilized and include an unmethylated CpG dinucleotide within certain preferred base contexts are potent at activating B cells but are relatively weak in inducing IFN-α and NK cell activation. See, e.g., U.S. Pat. Nos. 6,194,388; 6,207,646; 6,214,806; 6,218,371; 6,239,116; and 6,339,068.

In one embodiment a B class CpG oligonucleotide is represented by at least the formula:

5′ X₁X₂CGX₃X₄ 3′

wherein X₁, X₂, X₃, and X₄ are nucleotides. In one embodiment X₂ is adenine, guanine, or thymine. In another embodiment X₃ is cytosine, adenine, or thymine.

In another embodiment the invention provides an isolated B class CpG oligonucleotide represented by at least the formula:

5′ N₁X₁X₂CGX₃X₄N₂ 3′

wherein X₁, X₂, X₃, and X₄ are nucleotides and N is any nucleotide and N₁ and N₂ are nucleic acid sequences composed of from about 0-25 N's each. In one embodiment X₁X₂ is a dinucleotide selected from the group consisting of: GpT, GpG, GpA, ApA, ApT, ApG, CpT, CpA, CpG, TpA, TpT, and TpG; and X₃X₄ is a dinucleotide selected from the group consisting of: TpT, ApT, TpG, ApG, CpG, TpC, ApC, CpC, TpA, ApA, and CpA. Preferably X₁X₂ is GpA or GpT and X₃X₄ is TpT. In other embodiments X₁ or X₂ or both are purines and X₃ or X₄ or both are pyrimidines or X₁X₂ is GpA and X₃ or X₄ or both are pyrimidines. In another preferred embodiment X₁X₂ is a dinucleotide selected from the group consisting of: TpA, ApA, ApC, ApG, and GpG. In yet another embodiment X₃X₄ is a dinucleotide selected from the group consisting of: TpT, TpA, TpG, ApA, ApG, GpA, and CpA. X₁X₂ in another embodiment is a dinucleotide selected from the group consisting of: TpT, TpG, ApT, GpC, CpC, CpT, TpC, GpT and CpG; X₃ is a nucleotide selected from the group consisting of A and T and X₄ is a nucleotide, but wherein when X₁X₂ is TpC, GpT, or CpG, X₃X₄ is not TpC, ApT or ApC.

In another preferred embodiment the CpG oligonucleotide has the sequence 5′ TCN₁TX₁X₂CGX₃X₄ 3′. The CpG oligonucleotides of the invention in some embodiments include X₁X₂ selected from the group consisting of GpT, GpG, GpA and ApA and X₃X₄ is selected from the group consisting of TpT, CpT and TpC.

The C class immunostimulatory nucleic acids contain at least two distinct motifs have unique and desirable stimulatory effects on cells of the immune system. Some of these ODN have both a traditional “stimulatory” CpG sequence and a “GC-rich” or “B-cell neutralizing” motif. These combination motif nucleic acids have immune stimulating effects that fall somewhere between those effects associated with traditional “class B” CpG ODN, which are strong inducers of B cell activation and dendritic cell (DC) activation, and those effects associated A-class CpG ODN which are strong inducers of IFN-α and natural killer (NK) cell activation but relatively poor inducers of B-cell and DC activation. Krieg A M et al. (1995) Nature 374:546-9; Ballas Z K et al. (1996) J Immunol 157:1840-5; Yamamoto S et al. (1992) J Immunol 148:4072-6. While preferred class B CpG ODN often have phosphorothioate backbones and preferred class A CpG ODN have mixed or chimeric backbones, the C class of combination motif immune stimulatory nucleic acids may have either stabilized, e.g., phosphorothioate, chimeric, or phosphodiester backbones, and in some preferred embodiments, they have semi-soft backbones.

The stimulatory domain or motif is defined by a formula: 5′ X₁DCGHX₂ 3′. D is a nucleotide other than C. C is cytosine. G is guanine. H is a nucleotide other than G.

X₁ and X₂ are any nucleic acid sequence 0 to 10 nucleotides long. X₁ may include a CG, in which case there is preferably a T immediately preceding this CG. In some embodiments DCG is TCG. X₁ is preferably from 0 to 6 nucleotides in length. In some embodiments X₂ does not contain any poly G or poly A motifs. In other embodiments the immunostimulatory nucleic acid has a poly-T sequence at the 5′ end or at the 3′ end. As used herein, “poly-A” or “poly-T” shall refer to a stretch of four or more consecutive A's or T's respectively, e.g., 5′ AAAA 3′ or 5′ TTTT 3′.

As used herein, “poly-G end” shall refer to a stretch of four or more consecutive G's, e.g., 5′ GGGG 3′, occurring at the 5′ end or the 3′ end of a nucleic acid. As used herein, “poly-G nucleic acid” shall refer to a nucleic acid having the formula 5′ X₁X₂GGGX₃X₄ 3′ wherein X₁, X₂, X₃, and X₄ are nucleotides and preferably at least one of X₃ and X₄ is a G.

Some preferred designs for the B cell stimulatory domain under this formula comprise TTTTTCG, TCG, TTCG, TTTCG, TTTTCG, TCGT, TTCGT, TTTCGT, TCGTCGT.

The second motif of the nucleic acid is referred to as either P or N and is positioned immediately 5′ to X₁ or immediately 3′ to X₂.

N is a B-cell neutralizing sequence that begins with a CGG trinucleotide and is at least 10 nucleotides long. A B-cell neutralizing motif includes at least one CpG sequence in which the CG is preceded by a C or followed by a G (Krieg A M et al. (1998) Proc Natl Acad Sci USA 95:12631-12636) or is a CG containing DNA sequence in which the C of the CG is methylated. As used herein, “CpG” shall refer to a 5′ cytosine (C) followed by a 3′ guanine (G) and linked by a phosphate bond. At least the C of the 5′ CG 3′ must be unmethylated. Neutralizing motifs are motifs which has some degree of immunostimulatory capability when present in an otherwise non-stimulatory motif, but, which when present in the context of other immunostimulatory motifs serve to reduce the immunostimulatory potential of the other motifs.

P is a GC-rich palindrome containing sequence at least 10 nucleotides long. As used herein, “palindrome” and, equivalently, “palindromic sequence” shall refer to an inverted repeat, i.e., a sequence such as ABCDEE′D′C′B′A′ in which A and A′, B and B′, etc., are bases capable of forming the usual Watson-Crick base pairs.

As used herein, “GC-rich palindrome” shall refer to a palindrome having a base composition of at least two-thirds G's and C's. In some embodiments the GC-rich domain is preferably 3′ to the “B cell stimulatory domain”. In the case of a 10-base long GC-rich palindrome, the palindrome thus contains at least 8 G's and C's. In the case of a 12-base long GC-rich palindrome, the palindrome also contains at least 8 G's and C's. In the case of a 14-mer GC-rich palindrome, at least ten bases of the palindrome are G's and C's. In some embodiments the GC-rich palindrome is made up exclusively of G's and C's.

In some embodiments the GC-rich palindrome has a base composition of at least 81% G's and C's. In the case of such a 10-base long GC-rich palindrome, the palindrome thus is made exclusively of G's and C's. In the case of such a 12-base long GC-rich palindrome, it is preferred that at least ten bases (83%) of the palindrome are G's and C's. In some preferred embodiments, a 12-base long GC-rich palindrome is made exclusively of G's and C's. In the case of a 14-mer GC-rich palindrome, at least twelve bases (86%) of the palindrome are G's and C's. In some preferred embodiments, a 14-base long GC-rich palindrome is made exclusively of G's and C's. The C's of a GC-rich palindrome can be unmethylated or they can be methylated.

In general this domain has at least 3 Cs and Gs, more preferably 4 of each, and most preferably 5 or more of each. The number of Cs and Gs in this domain need not be identical. It is preferred that the Cs and Gs are arranged so that they are able to form a self-complementary duplex, or palindrome, such as CCGCGCGG. This may be interrupted by As or Ts, but it is preferred that the self-complementarity is at least partially preserved as for example in the motifs CGACGTTCGTCG (SEQ ID NO: 2) or CGGCGCCGTGCCG (SEQ ID NO: 3). When complementarity is not preserved, it is preferred that the non-complementary base pairs be TG. In a preferred embodiment there are no more than 3 consecutive bases that are not part of the palindrome, preferably no more than 2, and most preferably only 1. In some embodiments the GC-rich palindrome includes at least one CGG trimer, at least one CCG trimer, or at least one CGCG tetramer.

Spherical nucleic acids (SNAs) are a class of well-defined macromolecules, formed by organizing nucleic acids radially around a nanoparticle core, i.e., an inorganic metallic core (Mirkin C A, Letsinger R L, Mucic R C, & Storhoff J J (1996), A DNA-based method for rationally assembling nanoparticles into macroscopic materials. Nature 382(6592):607-609.). These structures exhibit the ability to enter cells without the need for auxiliary delivery vehicles or transfection reagents by engaging class A scavenger receptors (SR-A) and lipid rafts (Patel P C, et al. (2010) Scavenger receptors mediate cellular uptake of polyvalent oligonucleotide-functionalized gold nanoparticles. Bioconjugate chemistry 21(12):2250-2256.). Once inside the cell, the nucleic acid components of traditional SNAs resist nuclease degradation, leading to longer intracellular lifetimes. Moreover, SNAs, due to their multi-functional chemical structures, have the ability to bind their targets in a multivalent fashion (Choi C H, Hao L, Narayan S P, Auyeung E, & Mirkin C A (2013) Mechanism for the endocytosis of spherical nucleic acid nanoparticle conjugates. Proceedings of the National Academy of Sciences of the United States of America 110(19):7625-7630; Wu X A, Choi C H, Zhang C, Hao L, & Mirkin C A (2014) Intracellular fate of spherical nucleic acid nanoparticle conjugates. Journal of the American Chemical Society 136(21):7726-7733).

It has been discovered herein that immunostimulatory oligonucleotides formulated as IS-SNA have enhanced cancer therapeutic properties. IS-SNAs have been developed according to the invention which incorporate a densely packed oligonucleotide shell around a solid and or lipid core. These unique molecules can be used to efficiently deliver the oligonucleotides and optionally other therapeutic or diagnostic reagents to a cell, and in particular to cells in an efficient manner, resulting in enhanced therapeutic responses. Molecules packaged in the SNAs will be taken up into cells via scavenger receptor-mediated endocytosis, resulting in efficient and fast endosomal accumulation.

The nanostructures of the invention are typically composed of nanoparticles having a core and a shell of oligonucleotides, which is formed by arranging CpG oligonucleotides such that they point radially outwards from the core. A hydrophobic (e.g. lipid) anchor group attached to either the 5′-or 3′-end of the oligonucleotide, depending on whether the oligonucleotides are arranged with the 5′-or 3′-end facing outward from the core preferably is used to embed the oligonucleotides to a lipid based nanoparticle. The anchor acts to drive insertion into the lipid nanoparticle and to anchor the oligonucleotides to the lipids.

In some embodiments at least 25, 50, 75, 100, 200, 300, 400, 500, 600, 700, 800, 900 or 1,000 immunostimulatory oligonucleotides of the oligonucleotide shell or any range combination thereof are on the exterior of the core. In some embodiments, the oligonucleotide shell has a density of 1-1,000, 5-1,000, 100-1,000, 500-1,000, 10-500, 50-250, or 50-300 oligonucleotides per SI-SNA.

In some embodiments, the immunostimulatory oligonucleotides of the oligonucleotide shell are structurally identical immunostimulatory oligonucleotides. In other embodiments, the immunostimulatory oligonucleotides of the oligonucleotide shell have at least two structurally different immunostimulatory oligonucleotides. In certain embodiments, the immunostimulatory oligonucleotides of the oligonucleotide shell have 2-50, 2-40, 2-30, 2-10 or 2-10 different nucleotide sequences.

In some embodiments, at least 60%, 70%, 80%, 90%, 95%, 96%, 97% 98% or 99% of the oligonucleotides are positioned on the surface of the nanostructure. An oligonucleotide shell is formed when at least 10% of the available surface area of the exterior surface of a liposomal core includes an immunostimulatory oligonucleotide. In some embodiments at least 20%, 30%, 40%, 50%, 60%, 70%, 80%, 90%, 95%, 96%, 97%, 98% or 99% or 100% of the available surface area of the exterior surface of the liposomal includes an immunostimulatory oligonucleotide. The immunostimulatory oligonucleotides of the oligonucleotide shell may be oriented in a variety of directions. In some embodiments the immunostimulatory oligonucleotides are oriented radially outwards.

In some embodiments, at least 10% of the immunostimulatory oligonucleotides in the oligonucleotide shell are attached to the nanoparticle through a lipid anchor group. The lipid anchor consists of a hydrophobic group that enables insertion and anchoring of the oligonucleotides or nucleic acids to the lipid membrane. In some embodiments, at least 20%, at least 30%, at least 40%, at least 50%, at least 60%, at least 70%, at least 80%, at least 90%, at least 95%, at least 99%, or 100% of the oligonucleotides in the oligonucleotide shell are attached to the lipid nanoparticle through a lipid anchor group. In some embodiments, the lipid anchor group is cholesterol. In other embodiments, the lipid anchor group is sterol, palmitoyl, dipalmitoyl, stearyl, distearyl, C16 alkyl chain, bile acids, cholic acid, taurocholic acid, deoxycholate, oleyl litocholic acid, oleoyl cholenic acid, glycolipids, phospholipids, sphingolipids, isoprenoids, such as steroids, vitamins, such as vitamin E, saturated fatty acids, unsaturated fatty acids, fatty acid esters or other lipids known in the art.

In some embodiments, the oligonucleotides have a linker between the oligonucleotide and the lipid anchor group. A non-limiting example of a linker is tetraethyleneglycol.

The nanostructure includes a core. The core may be a solid or a hollow core, such as a liposomal core. A solid core is a spherical shaped material that does not have a hollow center. The term spherical as used herein refers to a general shape and does not imply or is not limited to a perfect sphere or round shape. It may include imperfections.

Solid cores can be constructed from a wide variety of materials known to those skilled in the art including but not limited to: noble metals (gold, silver), transition metals (iron, cobalt) and metal oxides (silica). In addition, these cores may be inert, paramagnetic, or supramagentic. These solid cores can be constructed from either pure compositions of described materials, or in combinations of mixtures of any number of materials, or in layered compositions of materials. In addition, solid cores can be composed of a polymeric core such as amphiphilic block copolymers, hydrophobic polymers such as polystyrene, poly(lactic acid), poly(lactic co-glycolic acid), poly(glycolic acid), poly(caprolactone) and other biocompatible polymers known to those skilled in the art. The solid core preferably is surrounded by a lipid bilayer.

The core may alternatively be a hollow core, which has at least some space in the center region of a shell material. Hollow cores include liposomal cores. A liposomal core as used herein refers to a centrally located core compartment formed by a component of the lipids or phospholipids that form a lipid bilayer. “Liposomes” are artificial, self-closed vesicular structure of various sizes and structures, where one or several membranes encapsulate an aqueous core. Most typically liposome membranes are formed from lipid bilayers membranes, where the hydrophilic head groups are oriented towards the aqueous environment and the lipid chains are embedded in the lipophilic core. Liposomes can be formed as well from other amphiphilic monomeric and polymeric molecules, such as polymers, like block copolymers, or polypeptides. Unilamellar vesicles are liposomes defined by a single membrane enclosing an aqueous space. In contrast, oligo-or multilamellar vesicles are built up of several membranes. Typically, the membranes are roughly 4 nm thick and are composed of amphiphilic lipids, such as phospholipids, of natural or synthetic origin. Optionally, the membrane properties can be modified by the incorporation of other lipids such as sterols or cholic acid derivatives.

The lipid bilayer is composed of two layers of lipid molecules. Each lipid molecule in a layer is oriented substantially parallel to adjacent lipid bilayers, and two layers that form a bilayer have the polar ends of their molecules exposed to the aqueous phase and the non-polar ends adjacent to each other. The central aqueous region of the liposomal core may be empty or filled fully or partially with water, an aqueous emulsion, oligonucleotides, or other therapeutic or diagnostic agent such as an antimicrobial agent.

“Lipid” refers to its conventional sense as a generic term encompassing fats, lipids, alcohol-ether-soluble constituents of protoplasm, which are insoluble in water. Lipids usually consist of a hydrophilic and a hydrophobic moiety. In water lipids can self organize to form bilayers membranes, where the hydrophilic moieties (head groups) are oriented towards the aqueous phase, and the lipophilic moieties (acyl chains) are embedded in the bilayers core. Lipids can comprise as well two hydrophilic moieties (bola amphiphiles). In that case, membranes may be formed from a single lipid layer, and not a bilayer. Typical examples for lipids in the current context are fats, fatty oils, essential oils, waxes, steroid, sterols, phospholipids, glycolipids, sulpholipids, aminolipids, chromolipids, and fatty acids. The term encompasses both naturally occurring and synthetic lipids. Preferred lipids in connection with the present invention are: steroids and sterol, particularly cholesterol, phospholipids, including phosphatidyl, phosphatidylcholines and phosphatidylethanolamines and sphingomyelins. Where there are fatty acids, they could be about 12-24 carbon chains in length, containing up to 6 double bonds. The fatty acids are linked to the backbone, which may be derived from glycerol. The fatty acids within one lipid can be different (asymmetric), or there may be only 1 fatty acid chain present, e.g. lysolecithins. Mixed formulations are also possible, particularly when the non-cationic lipids are derived from natural sources, such as lecithins (phosphatidylcholines) purified from egg yolk, bovine heart, brain, liver or soybean.

The liposomal core can be constructed from one or more lipids known to those in the art including but not limited to: sphingolipids such as sphingosine, sphingosine phosphate, methylated sphingosines and sphinganines, ceramides, ceramide phosphates, 1-0 acyl ceramides, dihydroceramides, 2-hydroxy ceramides, sphingomyelin, glycosylated sphingolipids, sulfatides, gangliosides, phosphosphingolipids, and phytosphingosines of various lengths and saturation states and their derivatives, phospholipids such as phosphatidylcholines, lysophosphatidylcholines, phosphatidic acids, lysophosphatidic acids, cyclic LPA, phosphatidylethanolamines, lysophosphatidylethanolamines, phosphatidylglycerols, lysophosphatidylglycerols, phosphatidylserines, lysophosphatidylserines, phosphatidylinositols, inositol phosphates, LPI, cardiolipins, lysocardiolipins, bis(monoacylglycero) phosphates, (diacylglycero) phosphates, ether lipids, diphytanyl ether lipids, and plasmalogens of various lengths, saturation states, and their derivatives, sterols such as cholesterol, desmosterol, stigmasterol, lanosterol, lathosterol, diosgenin, sitosterol, zymosterol, zymostenol, 14-demethyl-lanosterol, cholesterol sulfate, DHEA, DHEA sulfate, 14-demethyl-14-dehydrlanosterol, sitostanol, campesterol, ether anionic lipids, ether cationic lipids, lanthanide chelating lipids, A-ring substituted oxysterols, B-ring substituted oxysterols, D-ring substituted oxysterols, side-chain substituted oxysterols, double substituted oxysterols, cholestanoic acid derivatives, fluorinated sterols, fluorescent sterols, sulfonated sterols, phosphorylated sterols, and polyunsaturated sterols of different lengths, saturation states, and their derivatives.

The oligonucleotides are positioned on the exterior of the core. An oligonucleotide that is positioned on the core is typically referred to as coupled to the core. Coupled may be direct or indirect. The oligonucleotides may be reversibly or irreversibly coupled to the core. Reversibly coupled compounds are associated with one another using a susceptible linkage. A susceptible linkage is one which is susceptible to separation under physiological conditions. For instance Watson crick base pairing is a susceptible linkage. Cleavable linkers are also susceptible linkages.

Thus the IS-SNA are useful in some aspects of the invention as a stand-alone therapy, a combination therapy or as a vaccine for the treatment of a subject having cancer. The IS-SNA can be administered with or without a checkpoint inhibitor or an antigen or other therapeutic for the treatment of cancer.

A subject having a cancer is a subject that has detectable cancerous cells. The cancer may be a malignant or non-malignant cancer. Cancers or tumors include but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, or colon carcinoma.

A subject shall mean a human or vertebrate animal including but not limited to a dog, cat, horse, cow, pig, sheep, goat, turkey, chicken, primate, e.g., monkey, and fish (aquaculture species), e.g. salmon. Thus, the invention can also be used to treat cancer and tumors in non-human subjects. Cancer is one of the leading causes of death in companion animals (i.e., cats and dogs).

As used herein, the term treat, treated, or treating when used with respect to a disorder such as cancer refers to a prophylactic treatment which increases the resistance of a subject to development of the disease or, in other words, decreases the likelihood that the subject will develop the disease as well as a treatment after the subject has developed the disease in order to fight the disease (e.g., reduce or eliminate the cancer) or prevent the disease from becoming worse.

The IS-SNA maybe modified to include a cancer antigen. Alternatively a cancer antigen may be administered in conjunction with the IS-SNA. The term antigen broadly includes any type of molecule which is recognized by a host immune system as being foreign. A cancer antigen as used herein is a compound, such as a peptide or protein, associated with a tumor or cancer cell surface and which is capable of provoking an immune response when expressed on the surface of an antigen presenting cell in the context of an MHC molecule. Cancer antigens can be prepared from cancer cells either by preparing crude extracts of cancer cells, for example, as described in Cohen, et al., 1994, Cancer Research, 54:1055, by partially purifying the antigens, by recombinant technology, or by de novo synthesis of known antigens. Cancer antigens include but are not limited to antigens that are recombinantly expressed, an immunogenic portion of, or a whole tumor or cancer. Such antigens can be isolated or prepared recombinantly or by any other means known in the art.

The IS-SNA may also be co-loaded with or administered in conjunction with an anti-cancer therapy. Anti-cancer therapies include cancer medicaments, radiation and surgical procedures. As used herein, a “cancer medicament” refers to a agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. For the purpose of this specification, cancer medicaments are classified as chemotherapeutic agents, immunotherapeutic agents, checkpoint inhibitors, cancer vaccines, hormone therapy, and biological response modifiers.

Additionally, the methods of the invention are intended to embrace the use of more than one cancer medicament along with the IS-SNA. As an example, where appropriate, the IS-SNA may be administered with both a chemotherapeutic agent, a checkpoint inhibitor, and an immunotherapeutic agent. Alternatively, the cancer medicament may embrace an immunotherapeutic agent and a cancer vaccine, or a chemotherapeutic agent and a cancer vaccine, or a chemotherapeutic agent, an immunotherapeutic agent and a cancer vaccine all administered to one subject for the purpose of treating a subject having a cancer or at risk of developing a cancer.

The chemotherapeutic agent may be selected from the group consisting of methotrexate, vincristine, adriamycin, cisplatin, non-sugar containing chloroethylnitrosoureas, 5-fluorouracil, mitomycin C, bleomycin, doxorubicin, dacarbazine, taxol, fragyline, Meglamine GLA, valrubicin, carmustaine and poliferposan, MMI270, BAY 12-9566, RAS famesyl transferase inhibitor, famesyl transferase inhibitor, MMP, MTA/LY231514, LY264618/Lometexol, Glamolec, CI-994, TNP-470, Hycamtin/Topotecan, PKC412, Valspodar/PSC833, Novantrone/Mitroxantrone, Metaret/Suramin, Batimastat, E7070, BCH-4556, CS-682, 9-AC, AG3340, AG3433, Incel/VX-710, VX-853, ZD0101, ISI641, ODN 698, TA 2516/Marmistat, BB2516/Marmistat, CDP 845, D2163, PD183805, DX8951f, Lemonal DP 2202, FK 317, Picibanil/OK-432, AD 32/Valrubicin, Metastron/strontium derivative, Temodal/Temozolomide, Evacet/liposomal doxorubicin, Yewtaxan/Paclitaxel, Taxol/Paclitaxel, Xeload/Capecitabine, Furtulon/Doxifluridine, Cyclopax/oral paclitaxel, Oral Taxoid, SPU-077/Cisplatin, HMR 1275/Flavopiridol, CP-358 (774)/EGFR, CP-609 (754)/RAS oncogene inhibitor, BMS-182751/oral platinum, UFT (Tegafur/Uracil), Ergamisol/Levamisole, Eniluracil/776C85/5FU enhancer, Campto/Levamisole, Camptosar/Irinotecan, Tumodex/Ralitrexed, Leustatin/Cladribine, Paxex/Paclitaxel, Doxil/liposomal doxorubicin, Caelyx/liposomal doxorubicin, Fludara/Fludarabine, Pharmarubicin/Epirubicin, DepoCyt, ZD1839, LU 79553/Bis-Naphtalimide, LU 103793/Dolastain, Caetyx/liposomal doxorubicin, Gemzar/Gemcitabine, ZD 0473/Anormed, YM 116, lodine seeds, CDK4 and CDK2 inhibitors, PARP inhibitors, D4809/Dexifosamide, Ifes/Mesnex/Ifosamide, Vumon/Teniposide, Paraplatin/Carboplatin, Plantinol/cisplatin, Vepeside/Etoposide, ZD 9331, Taxotere/Docetaxel, prodrug of guanine arabinoside, Taxane Analog, nitrosoureas, alkylating agents such as melphelan and cyclophosphamide, Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate, but it is not so limited.

The immunotherapeutic agent may be selected from the group consisting of Ributaxin, Herceptin, Quadramet, Panorex, IDEC-Y2B8, BEC2, C225, Oncolym, SMART M195, ATRAGEN, Ovarex, Bexxar, LDP-03, ior t6, MDX-210, MDX-11, MDX-22, OV103, 3622W94, anti-VEGF, Zenapax, MDX-220, MDX-447, MELIMMUNE-2, MELIMMUNE-1, CEACIDE, Pretarget, NovoMAb-G2, TNT, Gliomab-H, GNI-250, EMD-72000, LymphoCide, CMA 676, Monopharm-C, 4B5, ior egf.r3, ior c5, BABS, anti-FLK-2, MDX-260, ANA Ab, SMART 1D10 Ab, SMART ABL 364 Ab and ImmuRAIT-CEA, but it is not so limited.

The cancer vaccine may be selected from the group consisting of EGF, Anti-idiotypic cancer vaccines, Gp75 antigen, GMK melanoma vaccine, MGV ganglioside conjugate vaccine, Her2/neu, Ovarex, M-Vax, O-Vax, L-Vax, STn-KHL theratope, BLP25 (MUC-1), liposomal idiotypic vaccine, Melacine, peptide antigen vaccines, toxin/antigen vaccines, MVA-based vaccine, PACIS, BCG vacine, TA-HPV, TA-CIN, DISC-virus and ImmuCyst/TheraCys, but it is not so limited.

The use of IS-SNA in conjunction with immunotherapeutic agents such as monoclonal antibodies is able to increase long-term survival through a number of mechanisms including significant enhancement of ADCC, activation of natural killer (NK) cells and an increase in IFNo levels. The IS-SNA when used in combination with monoclonal antibodies serve to reduce the dose of the antibody required to achieve a biological result.

The formulations of the invention are administered in pharmaceutically acceptable solutions, which may routinely contain pharmaceutically acceptable concentrations of salt, buffering agents, preservatives, compatible carriers, adjuvants, and optionally other therapeutic ingredients.

For use in therapy, an effective amount of the IS-SNA can be administered to a subject by any mode that delivers the IS-SNA to the desired surface, e.g., mucosal, systemic. Administering the pharmaceutical composition of the present invention may be accomplished by any means known to the skilled artisan. Preferred routes of administration include but are not limited to oral, parenteral, intramuscular, intranasal, sublingual, intratracheal, inhalation, ocular, vaginal, and rectal. In some embodiments preferred routes include intravenous injection, intratumoral injection and subcutaneous.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The IS-SNA and optionally other therapeutics and/or antigens may be administered per se (neat) or in the form of a pharmaceutically acceptable salt. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or alkaline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.004-0.02% w/v).

The pharmaceutical compositions of the invention contain an effective amount of a IS-SNA and optionally antigens and/or other therapeutic agents optionally included in a pharmaceutically-acceptable carrier. The term pharmaceutically-acceptable carrier means one or more compatible solid or liquid filler, diluents or encapsulating substances which are suitable for administration to a human or other vertebrate animal. The term carrier denotes an organic or inorganic ingredient, natural or synthetic, with which the active ingredient is combined to facilitate the application. The components of the pharmaceutical compositions also are capable of being commingled with the compounds of the present invention, and with each other, in a manner such that there is no interaction which would substantially impair the desired pharmaceutical efficiency.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

### EXAMPLES

### Example 1. TLR9-Targeted Spherical Nucleic Acids Show Potent Antitumor Activity in Syngeneic Tumor Models as Monotherapy and in Combination with an Anti-PD-1 Antibody

### Results

### Experiment 1: Subcutaneous and Intratumoral Administration of IS-SNA (CT26 Tumor)

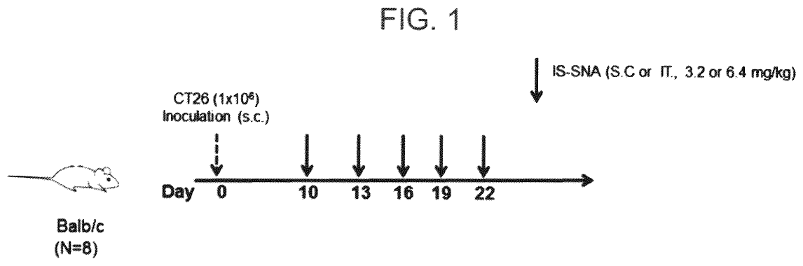

IS-SNA was intratumorally administered to the CT26 tumor (size ˜100 mm³) bearing Balb/c mice. IS-SNA was dosed at 3.2 or 6.4 mg/kg on days 10, 13, 16, 19 and 22 (FIG. 1). Tumor volumes were measured twice per week until the tumor size reaches 2000 mm³. The results indicate that both intratumoral and subcutaneous delivery of IS-SNA exhibited strong antitumor effects in a dose-dependent manner. The results also indicate the increased survival of the mice with increased IS-SNA dose.

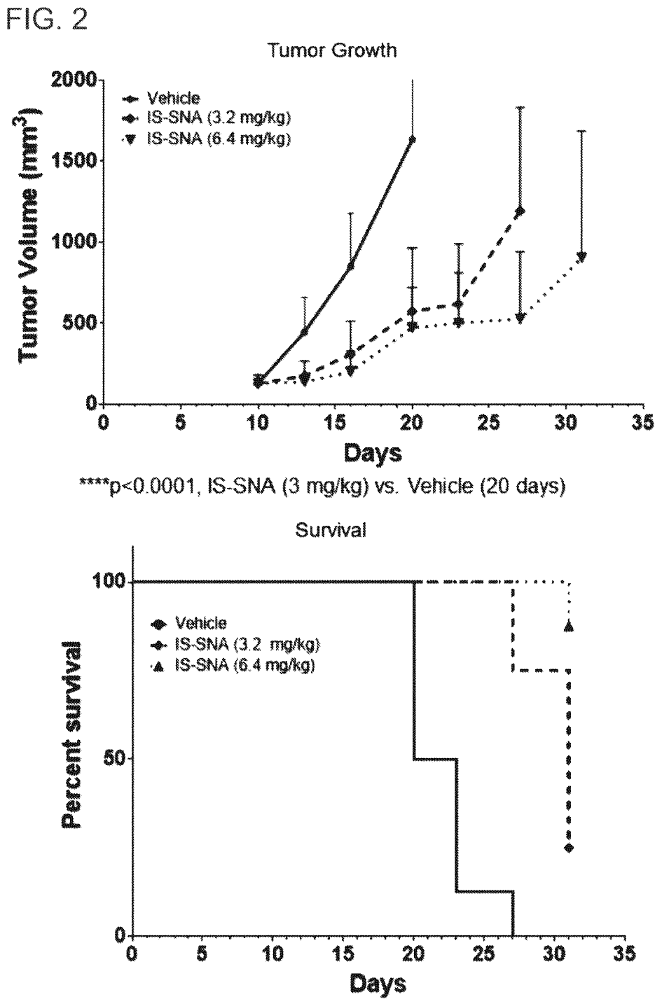

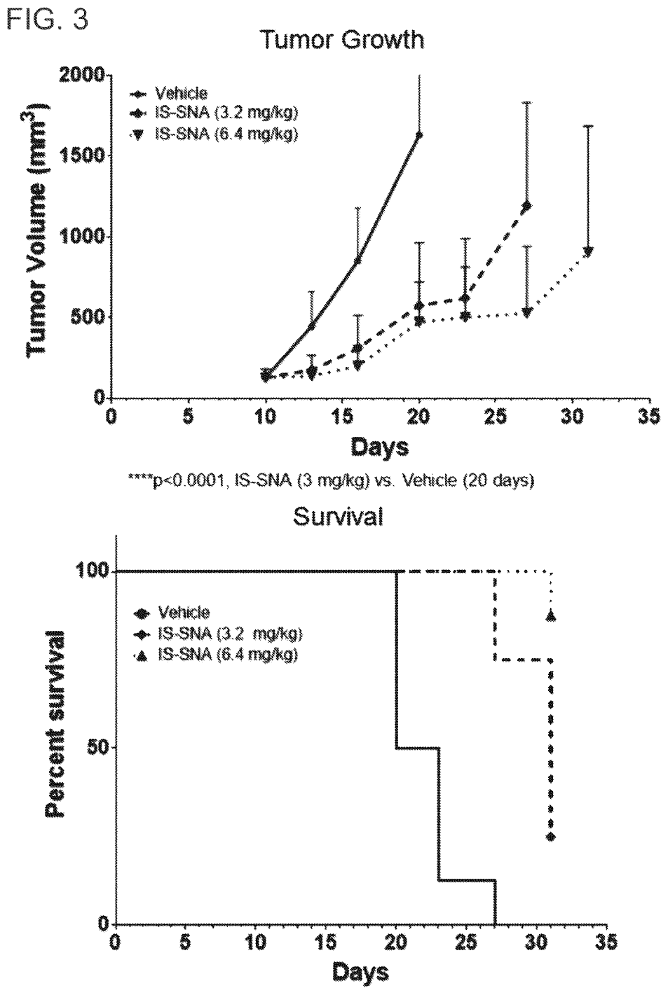

IS-SNA showed similar levels of antitumor effects for either subcutaneous (FIG. 2) or intratumoral delivery (FIG. 3).

### Experiment 2: Intratumoral Administration of IS-SNA (MC38 Tumor)

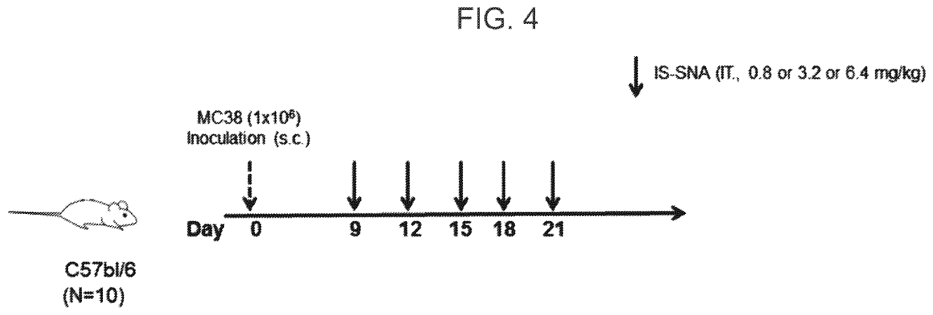

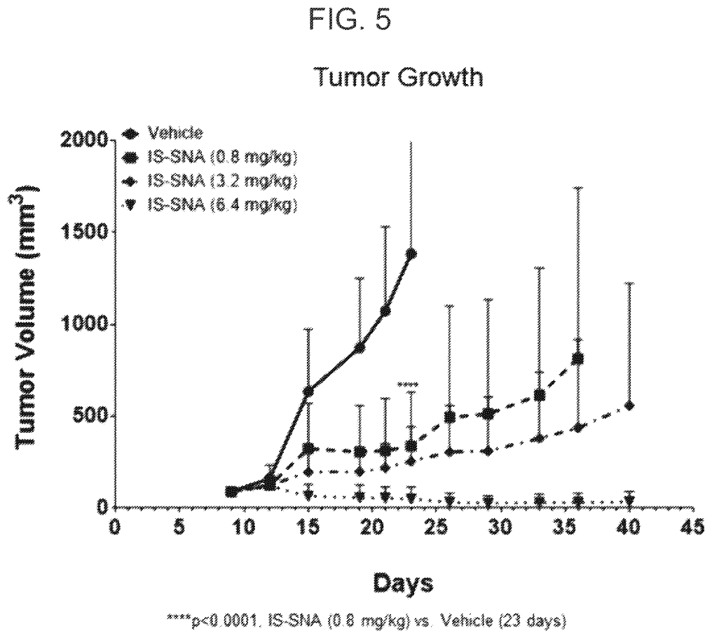

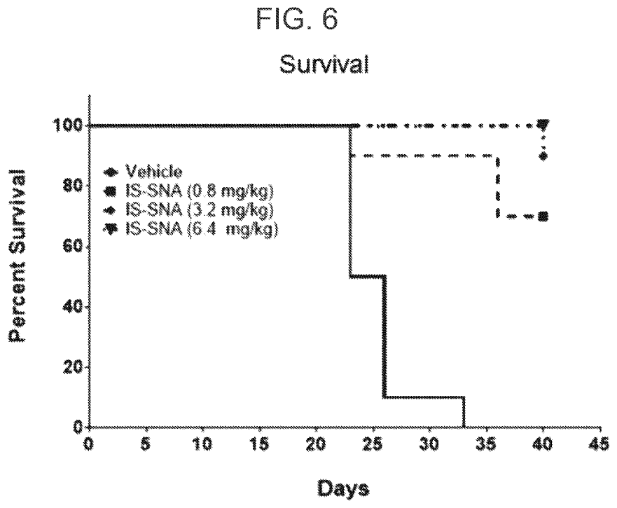

IS-SNA was intratumorally administered to the C57bl/6 mice bearing MC38 tumor of ˜100 mm³. IS-SNA was dosed at 0.8, 3.2 or 6.4 mg/kg on days 9, 12, 15, 18 and 21 (FIG. 4). Tumor volumes were measured twice per week until the tumor size reached 2000 mm³. The results indicate that IS-SNA exhibited potent antitumor effects in a dose dependent manner. IS-SNA was able to completely regress MC38 tumor growth at 6.4 mg/kg dose (FIG. 5). The results also indicate the increased survival of the mice in a dose-dependent manner (FIG. 6).

Experiment 3: IS-SNA Intravenously Administered in Combination with PD-1 (EMT-6 Tumor)

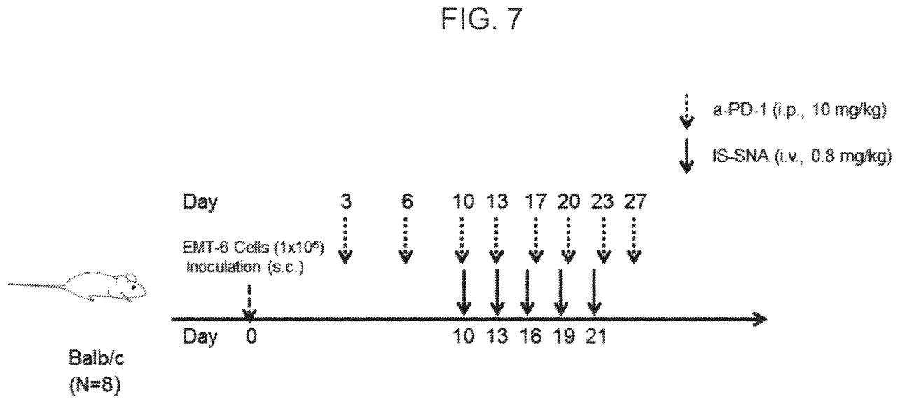

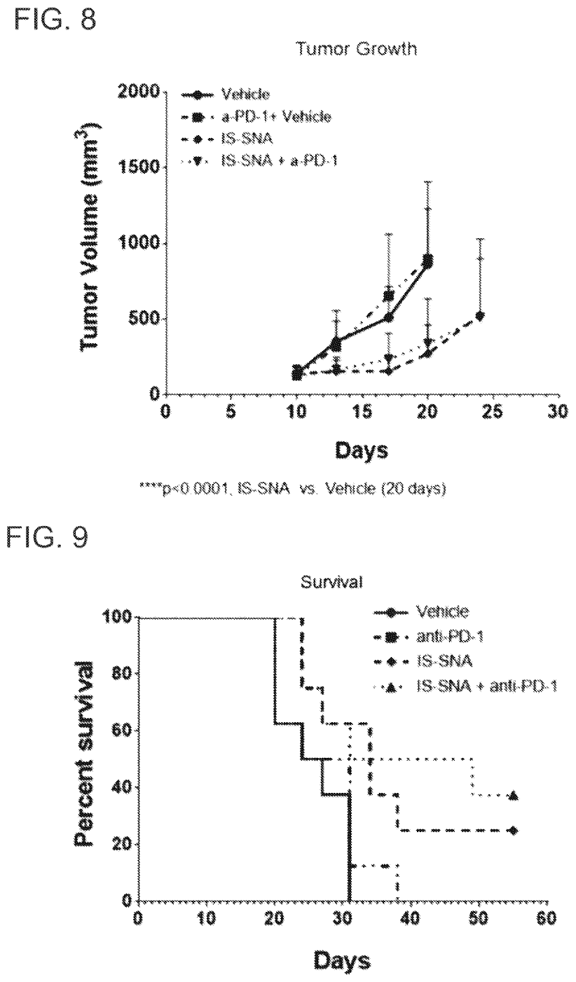

IS-SNA antitumor effects were monitored as a monotherapy and in combination with checkpoint inhibitor, a-PD-1, in Balb/c mice bearing ˜100 mm³ size tumors of EMT-6 breast cancer. IS-SNA was administered intravenously (IV) at 0.8 mg/kg on days 10, 13, 16, 19 and 21, and a-PD-1 was given intraperitoneally at 10 mg/kg on days 3, 6, 10, 13, 17, 20, 23 and 27 (FIG. 7). Tumor volumes were measured twice per week until the tumor size reached 2000mm³. The results indicate that intravenous administration of IS-SNA, both alone and in combination with a checkpoint inhibitor, can exert strong antitumor responses (FIG. 8). In addition, IS-SNA and a-PD-1 combination group has enhanced animal survival than IS-SNA alone, suggesting synergistic effects of combination in a-PD-1 resistant EMT-6 breast cancer model (FIG. 9).

Experiment 4: IS-SNA Subcutaneously Administered in Combination with PD-1 (EMT-6 Tumor)

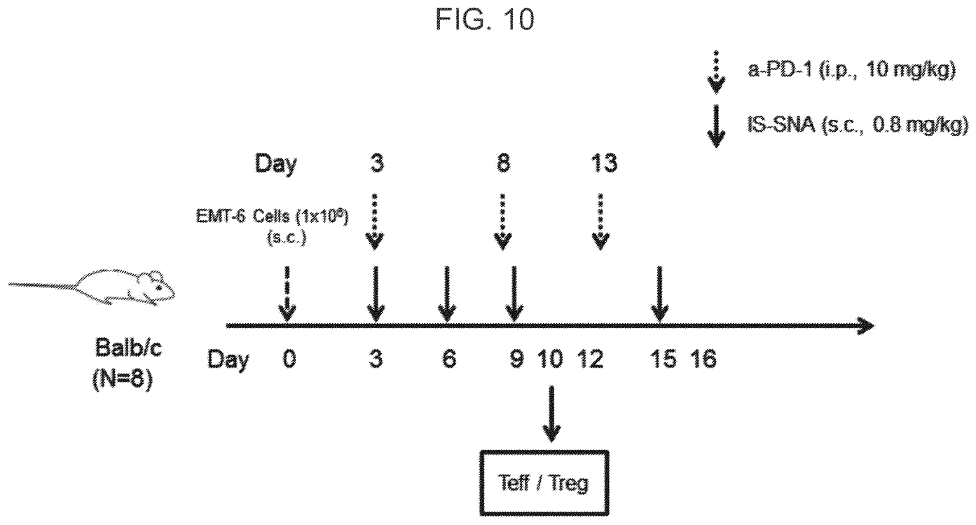

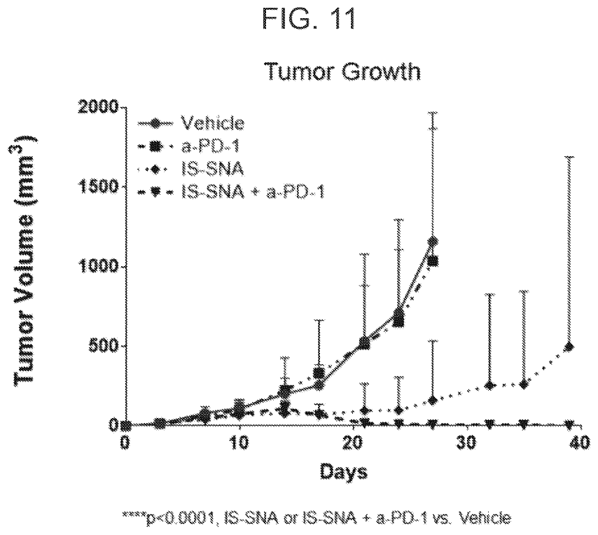

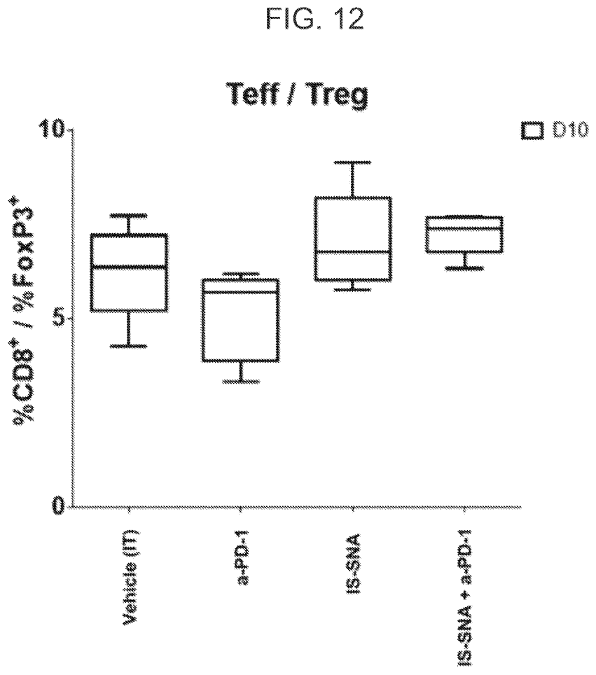

IS-SNA antitumor effects were monitored as a monotherapy and in combination with checkpoint inhibitor a-PD-1 in Balb/c mice bearing ˜4 mm³ size EMT-6 breast cancer tumors. IS-SNA was administered subcutaneously (peritumoral) around the tumor cell inoculation site at 0.8 mg/kg on days 3, 6, 9, 12 and 15, and a-PD-1 was given intraperitoneally at 10 mg/kg on days 3, 8 and 13 (FIG. 10). Tumor volumes were measured twice per week until the tumor size reached 2000 mm³. Ratios of T_{effectors}/T_{regulators} (T_{eff}/T_{reg}) were measured in draining lymph nodes of 5 animals on day 10 to probe the mechanistic understanding. The results suggest that subcutaneous administration of IS-SNA, both alone and in combination with checkpoint blockage, can exert strong antitumor responses. Combination of IS-SNA and a-PD-1 completely regressed the tumor growth in animals (FIG. 11). Mechanistic characterization results showed that mean values of T_{eff}/T_{reg} were higher for IS-SNA+a-PD-1 compared with IS-SNA alone suggesting higher antitumor effects of combination group was through the expected mechanism (FIG. 12).

Experiment 5: IS-SNA Subcutaneously Administered in Combination with PD-1 (B16F10 Tumor)

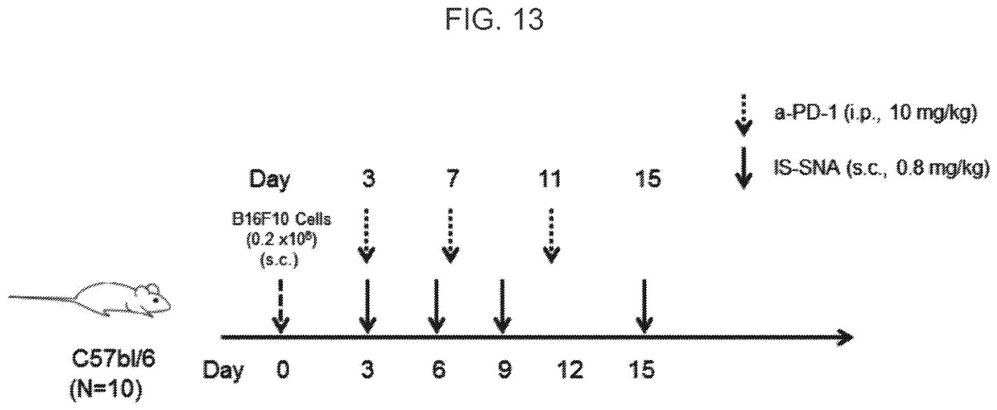

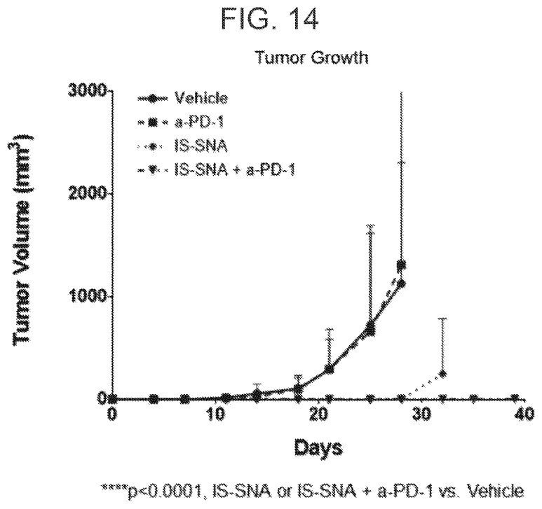

IS-SNA antitumor effects were monitored as a monotherapy and in combination with checkpoint inhibitor a-PD-1 in C57BL/6 mice bearing ˜4 mm³ size B16F10 melanoma tumors. IS-SNA was subcutaneously administered around the tumor cell inoculation site (peritumoral) at 0.8 mg/kg on days 3, 6, 9, 12 and 15, and a-PD-1 was given intraperitoneally at 10 mg/kg on days 3, 7, 11 and 15 (FIG. 13). Tumor volumes were measured twice per week until the tumor size reached 2000 mm³. The results suggest that subcutaneous administration of IS-SNA, both alone and in combination with checkpoint blockage, can exert potent antitumor responses. The combination of IS-SNA and a-PD-1 completely regressed the tumor growth in animals (FIG. 14).

### Materials and Methods

Oligonucleotide synthesis. Oligonucleotides were synthesized using automated solid support phosphoramidite synthesis. The IS-SNA sequence is 5-T*C*C*A*T*G*A*C*G*T*T*C*C*T*G*A*C*G*T*T-(SP18)-(SP18)-Cholesterol (SEQ ID NO: 1), *=‘PS’ substitution and SP18=Hexaethylene glycol spacer 18 molecule
Liposome synthesis. Liposomes were synthesized by extrusion of 1,2-dioleoyl-sn-glycero-3-phosphocholine (DOPC) hydrated in phosphate buffered saline solution (PBS) (137 mM NaCl, 10 mM phosphate, 2.7 mM KCl, pH 7.4, hyclone) using 47 mm diameter polycarbonate membranes with 50 nm pores (Sterlitech). Liposome diameters were measured using dynamic light scattering using a Malvern Zetasizer Nano (Malvern Instruments). DOPC concentration was determined using a phosphatidylcholine quantification kit (Sigma).
SNA synthesis (IS-SNA). To make SNAs, cholesterol-conjugated oligonucleotides were attached to the surface of the liposomes by mixing oligonucleotides to liposomes in a 100:1 ratio followed by incubation at room temperature for 4 h. Liposomes were then concentrated by TFF using a KrosFlo diafiltration system with 300-KDa dialysis membranes (Spectrum Labs). Liposome concentration was calculated using DOPC concentration, liposome diameter, and phosphatidylcholine head group area (0.71 nm²). Oligo concentration was determined with a UV spectrophotometer by dissolving liposomes in 100% methanol. This average loading was determined to be 100 oligonucleotides per liposome.
Mouse Tumor Models. For the CT26 model (experiment 1), 7 to 8-week-old female Balb/c mice (Charles River) were inoculated in the flank subcutaneously with 1×10⁶ CT26 tumor cells.

For the MC38 model (experiment 2), 7 to 8-week-old female C57BL/6 mice (Charles River) were inoculated in the flank subcutaneously with 1×10⁶ MC38 tumor cells.

For the EMT-6 model (experiments 3 and 4), 7 to 8-week-old Balb/c mice (Charles River) were inoculated in the flank subcutaneously with 1×10⁶ EMT-6 tumor cells.

For B16F10 model (experiment 5), 7 to 8-week-old female C57BL/6 mice (Charles River) were inoculated in the flank subcutaneously with 0.2×10⁶ B16F10 tumor cells. Tumor sizes were measured twice weekly in two dimension using a caliper, and the volumes presented in mm³ using the formula:

$$Tumor volume = ( {Width}^{2} \times Length ) / 2$$

Dosing schedules of IS-SNA and a-PD-1 (clone: RMP1-14, catalog: BE0146, isotype: Rat 2A3, Bioxcell) are shown in the schematic diagrams of the corresponding experiments. In the prevention models, IS-SNA was dosed starting on 3^{rd} day after tumor cell inoculation, whereas in established tumor models, dosing of IS-SNA was started when mean tumor volume of the groups reached 100 mm³ tumor sizes. In certain experiments, tumors and tumor-draining lymph nodes were harvested for the measurement of immune infiltrating cells. Statistical comparisons among groups were performed by ANOVA with Sidak's (Two-way ANOVA) post-hoc multiple comparisons using GraphPad Prism 6.05. Differences between groups were considered significant when p<0.05.

FACS Analysis. The immune infiltrate cells were characterized by FACS analysis from each collected sample. Briefly, the collected samples were processed by mechanical dissociation and prepared in 100 μL staining buffer (PBS, 0.2% BSA, 0.02% NaN₃). Then the antibodies directed against the chosen markers were added according to the procedure described by the supplier for each antibody.

The antibodies and their respective isotypes used for FACS analyses for the characterization of immune cells populations on mouse samples are listed in the tables below. The mixture was incubated for 20 to 30 minutes at room temperature in the dark, washed, and resuspended in 200 μL staining buffer. Samples were also processed with control isotype antibodies.

At the end of the incubation period, cells were washed with permeabilization buffer if necessary, centrifuged and resuspended in reference microbeads solution (PKH26, Ref P7458, Sigma, diluted at ½ in staining buffer). All samples were stored on ice and protected from light until FACS analysis. The stained cells were analyzed with a CyFlow® space flow cytometer (LSR II, BD Biosciences) equipped with 3 excitation lasers at wavelengths 405, 488 and 633 nm. FACS data was be acquired until either 10,000 mCD45+ events are recorded for each sample, or for a maximum duration of 2 minutes. All events were saved during acquisition.

TABLE 1
Antibodies used for analysis of myeloid-derived suppressor cells

| Reference | Target | Fluorochrome | Vendor |
|---|---|---|---|
| AM05612FC-N | CD274 (=PDL-1) | FITC | Acris/Interchim |
| 555843 | IgG2a | FITC | BD Biosciences |
| 553063 | CD3 | PE | BD Biosciences |
| 553972 | IgG1 | PE | BD Biosciences |
| 130-107-917 | Ly-6G | PerCP Vio770 | Miltenyi Biotec |
| 130-104-620 | REA Control (S) | PerCP Vio770 | Miltenyi Biotec |
| 25-5920-82 | INOS = NOS2 | PE-Cy7 | ebioscience |
| 552784 | IgG2a | PE-Cy7 | BD Biosciences |
| 563890 | CD45 | BV421 | BD Biosciences |
| 562603 | IgG2 | BV421 | BD Biosciences |
| 130-102-207 | Ly-6C | VioGreen | Miltenyi Biotec |
| 130-103-096 | IgG2a | VioGreen | Miltenyi Biotec |
| IC5868A | Arg1 | APC | R&D Systems |
| IC016A | IgG | APC | R&D Systems |
| 557657 | CD11b | APCCy7 | BD Biosciences |
| 552773 | IgG2b | APC-Cy7 | BD Biosciences |
| 130-090-477 | Inside Stain Kit | — | Miltenyi Biotec |

TABLE 2
Antibodies used for analysis of T cells

| Reference | Target | Fluorochrome | Vendor |
|---|---|---|---|
| 130-102-249 | PD-1 | FITC | Miltenyi Biotec |
| 553988 | IgG2b | FITC | BD Biosciences |
| 130-093-014 | FoxP3 | PE | Miltenyi Biotec |
| A07796 | IgG1 | PE | Beckman Coulter |
| 553036 | CD8a | PerCP | BD Biosciences |
| 553933 | IgG2a | PerCP | BD Biosciences |
| 552880 | CD25 | PE-Cy7 | BD Biosciences |
| 552869 | IgG1 | PE-Cy7 | BD Biosciences |
| 561389 | CD3 | V450 | BD Biosciences |
| 560457 | IgG2 | V450 | BD Biosciences |
| 130-102-444 | CD4 | VioGreen | Miltenyi Biotec |
| 130-102-659 | IgG2b | VioGreen | Miltenyi Biotec |
| 557659 | CD45 | APC-Cy7 | BD Biosciences |
| 552773 | IgG2b | APC-Cy7 | BD Biosciences |
| 130-102-340 | IFNg | APC | Miltenyi Biotec |
| 400412 | IgG1 | APC | biolegend |
| 130-093-142 | intracellular + Kit fox | | Miltenyi Biotec |

### References

1. A. M. Krieg, S. M. Efler, M. Wittpoth, M. J. Al Adhami, and H. L. Davis, ‘Induction of Systemic Th1-Like Innate Immunity in Normal Volunteers Following Subcutaneous but Not Intravenous Administration of Cpg 7909, a Synthetic B-Class Cpg Oligodeoxynucleotide Tlr9 Agonist’, J Immunother, 27 (2004), 460-71.
2. T. Okazaki, and T. Honjo, ‘The Pd-1-Pd-L Pathway in Immunological Tolerance’, Trends Immunol, 27 (2006), 195-201.
3. L. Pusztai, A. Ladanyi, B. Szekely, and M. Dank, ‘[Immunotherapy Opportunities in Breast Cancer]’, Magy Onkol, 60 (2016), 34-40.

### Example 2. TLR9-Targeted Spherical Nucleic Acids Induce Immune Responses in Monkeys and Anti-Tumor Immunity with an Anti-PD-1 Antibody in Mice Abstract

TLR9 agonists have been clinically evaluated for anti-tumor activity without much success. Spherical nucleic acids (SNAs) are novel agents based on dense spherical arrangement of oligonucleotides on a nanoparticle core, and overcome limitations of linear therapeutic oligonucleotides. TLR9 agonist SNAs increased cellular uptake and TLR9 activation in vitro compared with a linear oligonucleotide. In vivo, in mice and monkeys, SNAs induced higher TH1-type cytokines compared with a linear oligonucleotide. In murine tumor models, SNAs inhibited tumor growth and prolonged mouse survival. SNA and anti-PD-1 combination enhanced antitumor effects compared with either agent alone. SNA treated mice tumor tissue and draining lymph nodes showed increased cytotoxic T cells, and reduced Tregs and monocytic MDSC. Tumor re-challenge demonstrated tumor-specific immunological memory. These studies support TLR9 agonist SNAs as promising cancer immunotherapy as monotherapy and in combination with checkpoint inhibitors.

### Introduction

Recognition of pathogens and danger signals by the innate immune system is dependent upon pattern recognition receptors (PRR). Toll-like receptors (TLRs) are one of the classes of PRR. In humans, eleven TLRs, TLR1-11, have been identified. TLR9 is expressed in the endosomal compartments of human B cells and plasmacytoid dendritic cells (pDC). TLR9 recognizes bacterial and synthetic oligonucleotides (oligos) containing unmethylated CpG dinucleotides present in specific sequence contexts, referred to as CpG motifs (1-5). TLR9 stimulation by CpG oligonucleotides results in the production of T_{H}1-type innate and adaptive immune responses (6, 7). TLR9 agonists are classified into A-, B-, and C-class on the basis of sequence characteristics and specific immunostimulatory profiles they produce (8). All three types of TLR9 agonists have been extensively evaluated in preclinical (8-10) and clinical studies for cancer and infectious diseases (11).

The potential of TLR9 agonists to stimulate both innate and adaptive immune responses has captured the attention of the oncology community, and over three dozen clinical trials have been performed in cancer patients using TLR9 agonists. CpG 7909 (also known as ODN 2006, PF-3512676, and ProMune), which belongs to the B-class of TLR9 agonists, was most extensively studied (12). The TLR9 agonists evaluated to date, including CpG 7909, neither produced sufficient anti-tumor responses as a monotherapy nor improved efficacy when combined with other approved anticancer agents (12, 13) because of their poor cellular uptake.

SNAs are three-dimensional arrangements of nucleic acids, with densely packed oligonucleotides radially arranged on a central nanoparticle core (14, 15). The SNA platform is highly adaptable and can be used with a variety of nucleic acid classes including immunostimulatory and immunoregulatory oligonucleotides, antisense oligonucleotides, siRNA, and miRNA (16). Additionally, SNAs can be designed to include peptides, proteins, or targeting antibodies along with oligonucleotides on the nanoparticle (17-19). The central nanoparticle core functions as a structural element to form the SNA and can be composed of various materials including gold, silica, or a lipid bilayer (16). Unlike in other commonly used oligonucleotide delivery systems, such as encapsulation in cationic lipids, polymers, or liposomes, oligonucleotides on SNA are exposed externally and readily available for interaction with their targets, including transmembrane receptors such as TLR9. SNAs have been shown to be taken up by cells via scavenger receptors and delivered into the endosomes where TLR9 is expressed (20-22).

Taking advantage of SNA properties, TLR9 agonist oligonucleotides were formulated (Table 3) as SNAs around a neutral DOPC lipid core and their immunostimulatory profiles were assessed in vitro and in vivo in mice and non-human primates (NHPs), and antitumor efficacy in murine tumor models. TLR9 agonist SNAs showed specific activation of TLR9 in cell-based assays, induced T_{H}1-type cytokines in vitro and in vivo, and promoted anti-tumor immunity in murine tumor models both as a monotherapy and in combination with an anti-PD-1 checkpoint inhibitor (CPI). SNAs promoted antitumor immunity by increasing cytotoxic T-cells and reducing T-regulatory cells and monocytic myeloid-derived suppressor cells (mMDSCs) in the tumor microenvironment (TME) and draining lymph node (DLN) of SNA treated mice.

TABLE 3
Oligonucleotide sequences of SNAs and linear oligonucleotides. From top to
bottom, the compounds correspond to SEQ ID NOs: 4, 5, 6, 7, and 8.

| Name of compound | Oligonucleotide Sequence (5′→3′)* | Selectivity |
|---|---|---|
| SNA1 | TCGTCGTTTTGTCGTTTTGTCGTT-(SP18)₂-TEG-cholesterol | Human |
| Linear oligo 2 | TCGTCGTTTTGTCGTTTTGTCGTT | Human |
| SNA3 | TCCATGACGTTCCTGACGTT-(SP18)₂-TEG-cholesterol | Mouse |
| Linear oligo 4 | TCCATGACGTTCCTGACGTT | Mouse |
| Control SNA5 | TGCTGCTTTTGTGCTTTTGTGCTT-(SP18)₂-TEG-cholesterol | N/A |

*All sequences contain a phosphorothioate backbone; SP18 stands for spacer-18 or hexaethyleneglycol linker; TEG stands for tetraethyleneglycol linker; underline indicates CpG. For uptake studies fluorescein labeled SNA1 and linear oligo 2 were used, which were synthesized with a fluorescein label on the 3′-terminal thymidine.

### Results

### Increased Cellular Uptake of SNA Compared With Linear Oligonucleotide

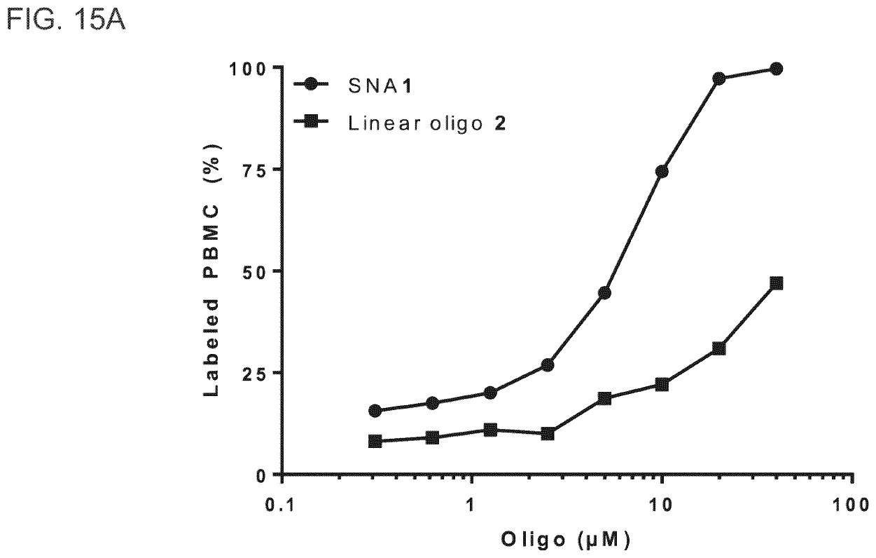

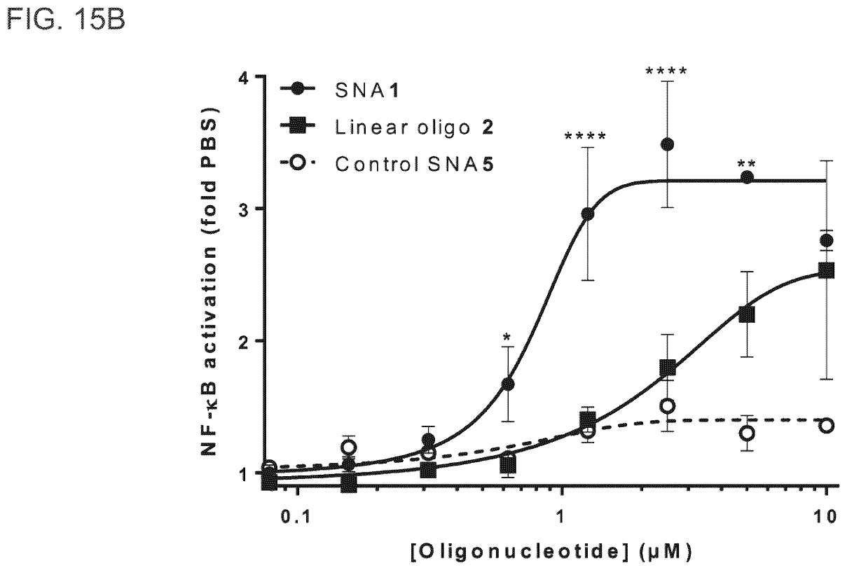

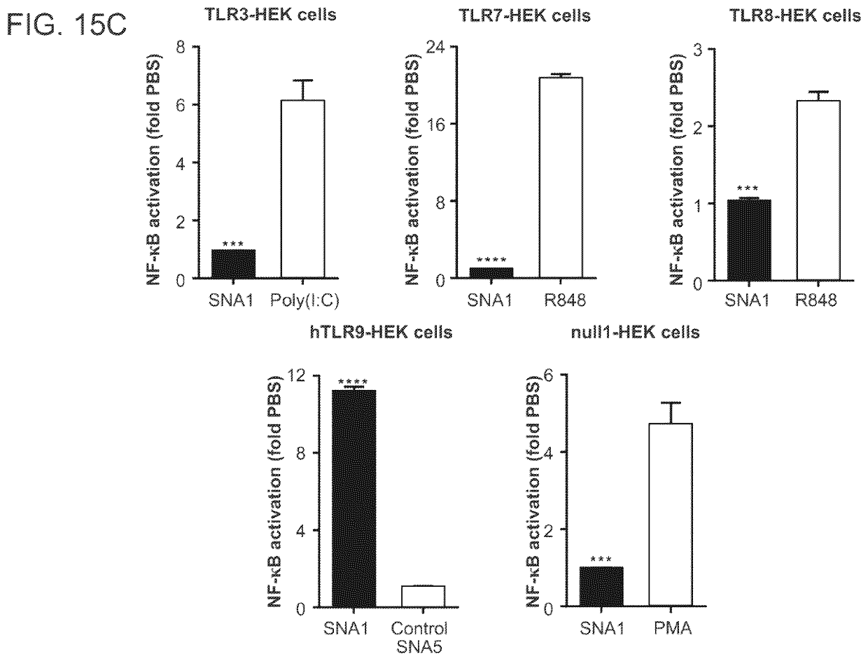

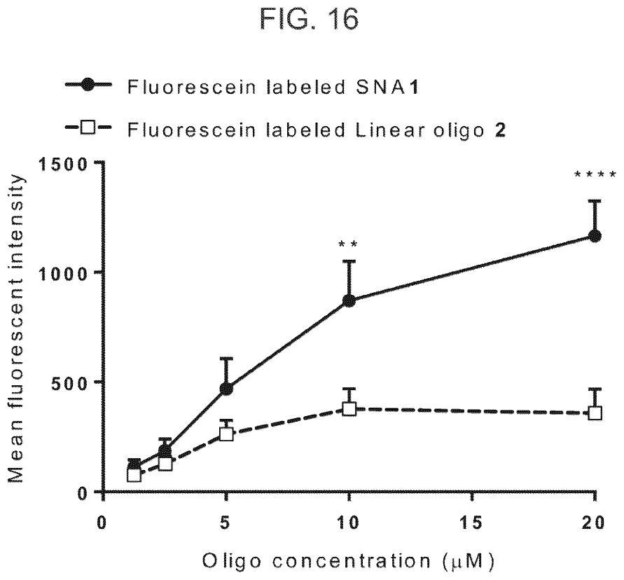

Cellular uptake of SNA and a linear oligonucleotide that is not in SNA format was studied by incubating human peripheral blood mononuclear cells (hPBMC) with fluorescently labeled SNA1 or linear oligo 2. As measured by flow cytometry, a larger fraction of PBMCs were fluorescein-positive after treatment with fluorescently labeled SNA1 than linear oligo 2 (FIG. 15A). Additionally, the mean fluorescent intensity of SNA-treated cells was greater, indicating that each cell took up a greater number of oligonucleotides when delivered as SNA format than as linear oligo (FIG. 16).

### Greater TLR9 Activation by SNA Compared With a Linear Oligonucleotide

TLR9 activation by SNA1 and linear oligo 2 was evaluated in HEK293 cells stably transfected with human TLR9. After four hours of incubation, TLR9 activation was about 2-fold greater with SNA1 than linear oligo 2 at a concentration of 1.25 μM (FIG. 15B). The measured EC₅₀ were 0.88 and 2.59 μM for SNA1 and linear oligo 2, respectively. The higher TLR9 activation with SNA1 compared with linear oligo 2 are consistent with increased cellular uptake of SNA as observed in the previous experiment.

### Specificity of TLR9 Agonist SNAs

To confirm that the stimulation by SNA was TLR9 specific, HEK293 reporter cells stably transfected with no TLR (null) or with human TLR3, TLR7, or TLR8, which recognize RNA-based nucleic acids, or TLR9, was used. Only HEK cells expressing TLR9 are stimulated by SNA1 (FIG. 15C). Control SNA5 in which CpG dinucleotides are replaced with GpC dinucleotides failed to activate TLR9, suggesting CpG dinucleotides in the SNA are required for efficient interaction and stimulation of TLR9. The HEK cells expressing TLR3, TLR7 or TLR8 are activated by their respective ligands, but not SNA1 (FIG. 15C) suggesting SNA1 does not stimulate these specific TLRs. Incubation of TLR null cells with SNA1 did not show any activation, further confirming that the stimulation by SNA1 is TLR9 specific.

### Cytokine Induction by TLR9 Agonist SNAs in Mouse and Human Primary Cell Cultures

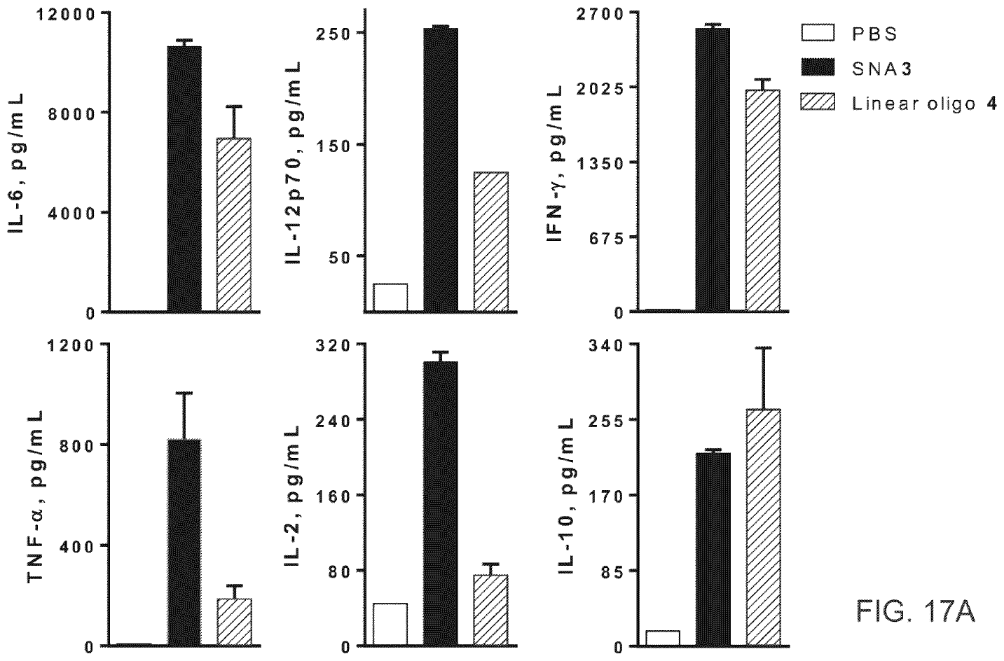

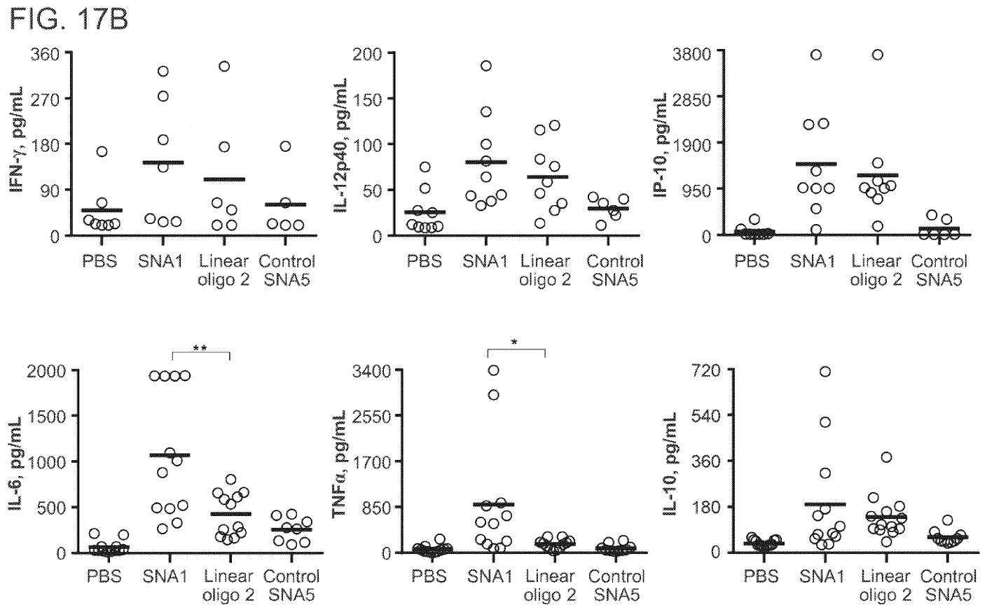

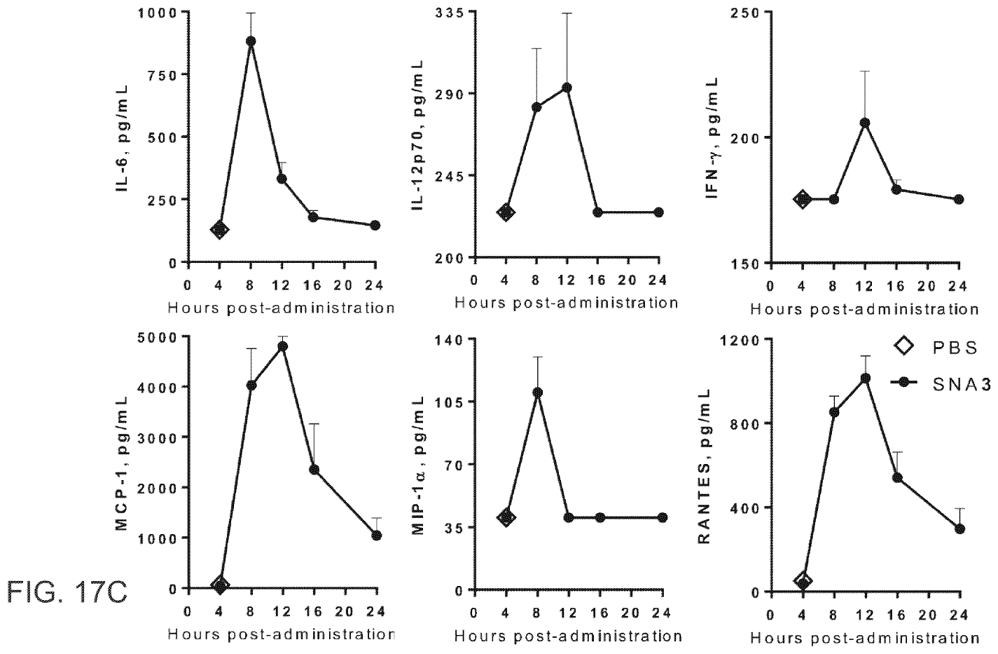

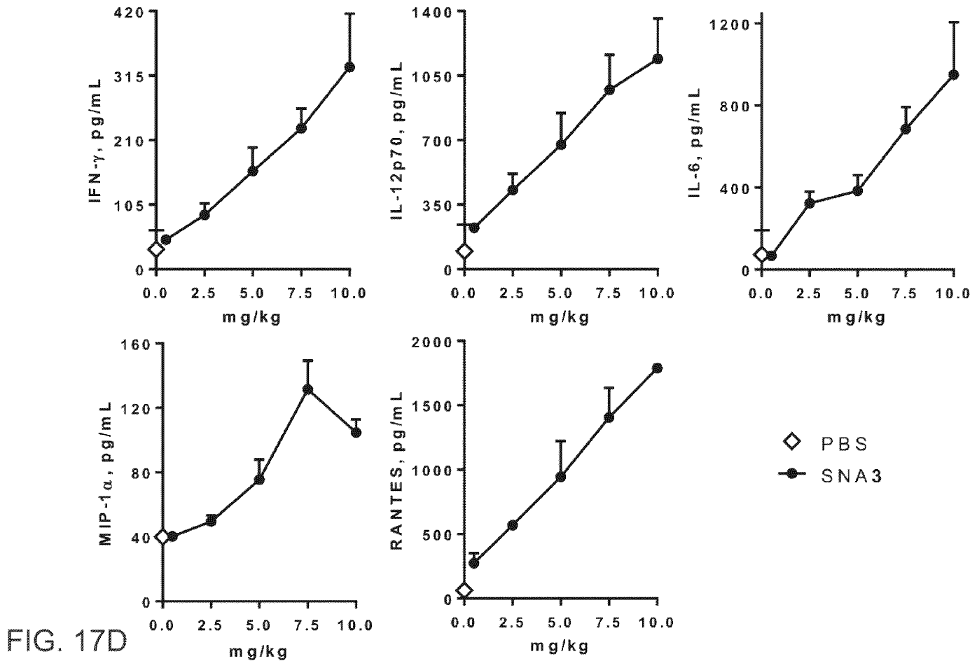

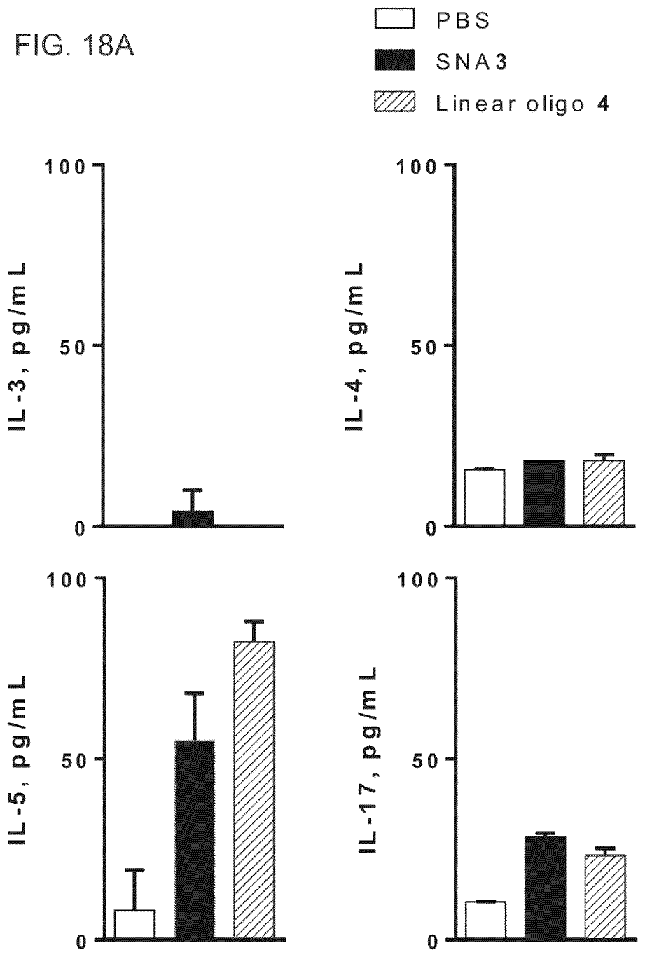

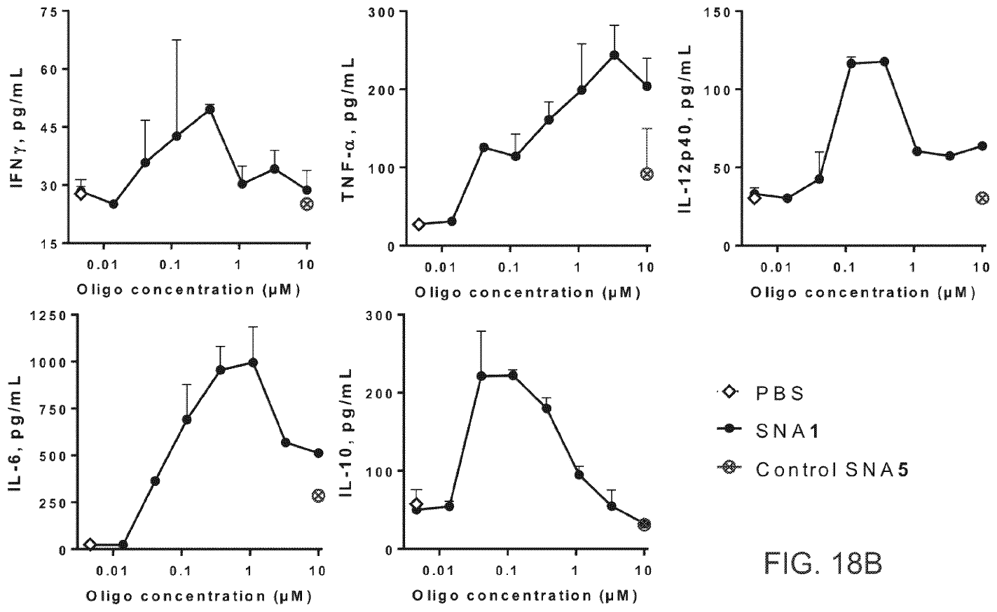

Having established greater cellular uptake and TLR9 specific activation of SNA agonists in cell lines, the cytokine profiles induced by TLR9 agonists in mouse splenocytes were then studied. When primary mouse splenocytes were incubated overnight with SNA3 or linear oligo 4, an increase in the levels of T_{H}1-type cytokines was observed, IL-2, IL-6, IL-12, IFN-γ, TNF-α, and IL-10 in the cell culture supernatants with both compounds (FIG. 17A). No or minimal T_{H}2-type cytokines such as IL-3, IL-4, IL-5, or IL-17 were observed (FIG. 18A). In general, a higher level of T_{H}1-type cytokines, except IL-10, was induced with SNA3 than linear oligo 4 in mouse splenocytes (FIG. 17A).

Similarly, experiments were carried out with human-specific SNA1 and linear oligo 2 in multiple healthy human volunteer PBMC cultures. In general, higher levels of T_{H}1-type cytokines, IL-6, IL-12, IFN-γ, TNF-α, IP-10, and IL-10 were induced in primary hPBMCs with SNA1 compared with linear oligo 2 (FIG. 17B). Control SNA5 showed background levels of cytokine induction similar to PBS control (FIG. 17B). Further, the cytokine induction in human PBMCs was dependent on the concentration of SNA1 used (FIG. 18B).

### Cytokine Induction by SNAs in Vivo in Mice

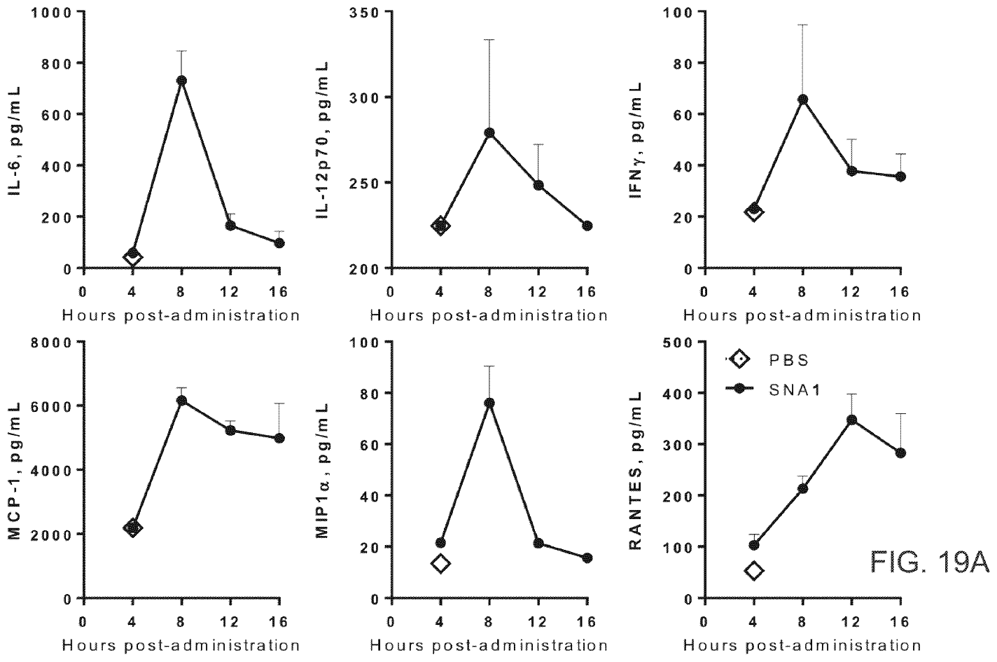

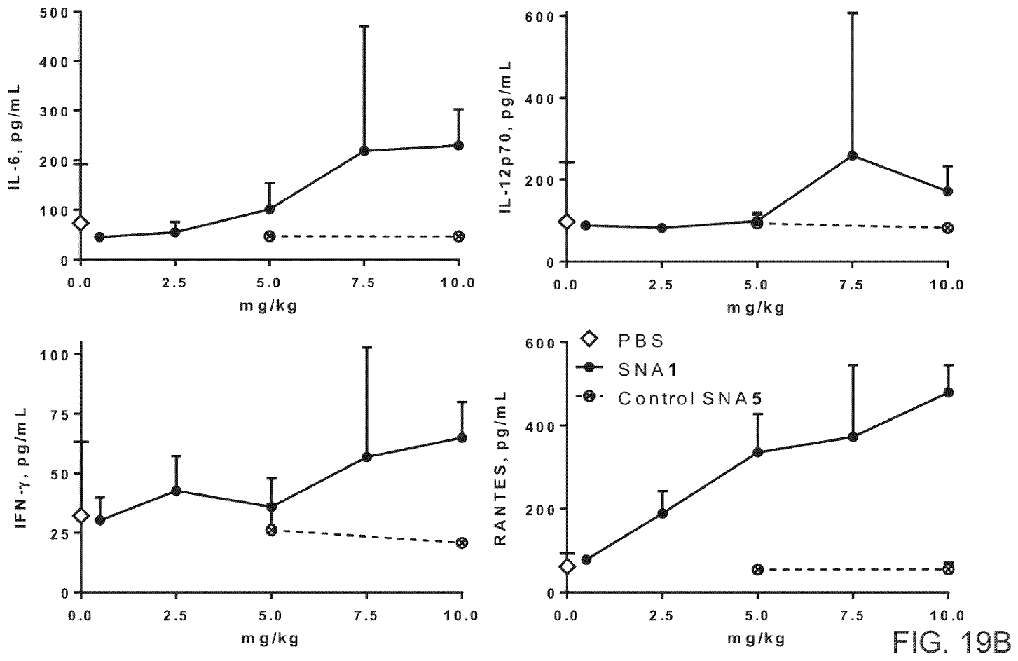

Next, the level, kinetics, and type of systemic cytokine induction following subcutaneous administration of SNA3 and linear oligo 4 to C57BL/6 mice was assessed. Both SNA3 and linear oligo 4 induced a systemic T_{H}1-type cytokine response in mice. The peak serum cytokine response to linear oligo TLR9 agonists occurred between 2 and 6 hr post administration as has been reported previously (23-25). However, the peak cytokine response to SNA3 occurred between 8 and 12 hr post administration (FIG. 17C). A similar time course of cytokine induction in mice was observed with human TLR9 selective SNA1 (FIG. 19A). SNA3 induced T_{H}1-type cytokines, IL-6, IL-12, and IFN-γ, and chemokines, MIP-1α, MCP-1 and RANTES, and the induction was dependent on the dose of SNA3 administered (FIG. 17D). SNA1 also showed dose-dependent cytokine induction in mice though to a lower extent as expected (FIG. 19B). Control SNA5 in which CpG dinucleotides are replaced with GpC dinucleotides did not stimulate a cytokine response (FIG. 19B), indicating that the presence of CpG dinucleotides is required for TLR9-mediated cytokine induction.

### Immune Response Profiles of TLR9 Agonist SNA in Vivo in Non-Human Primates

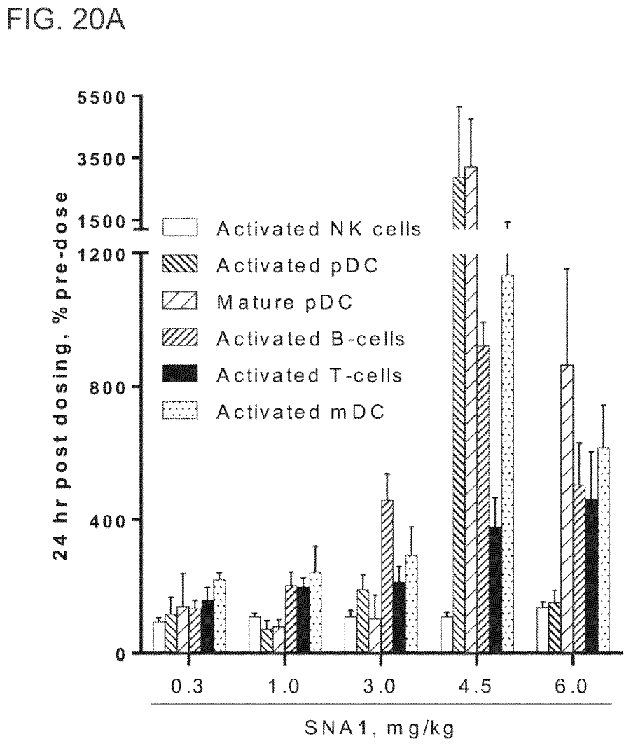

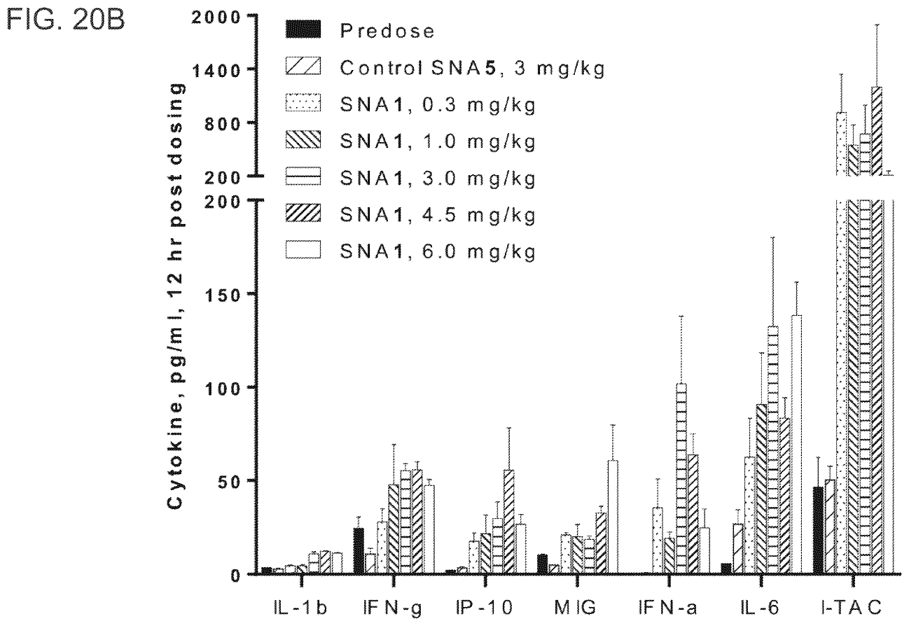

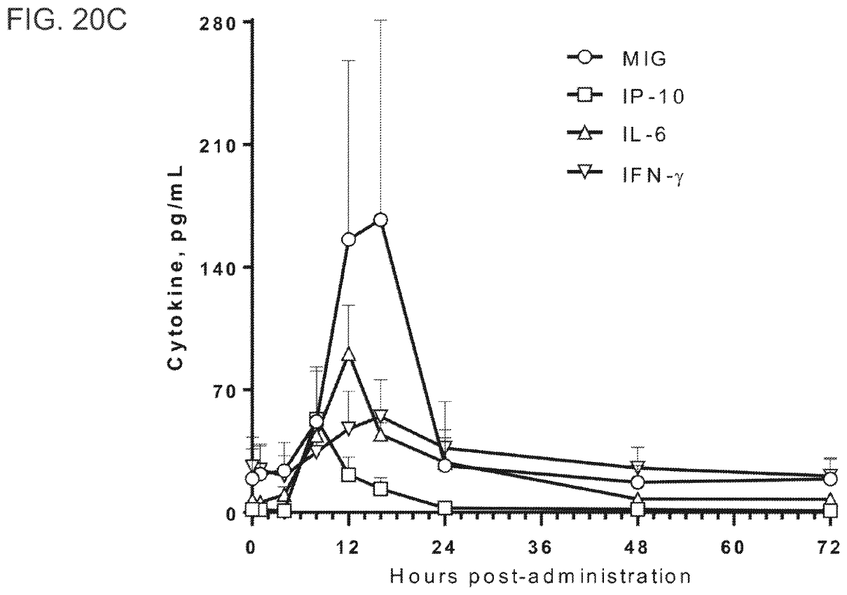

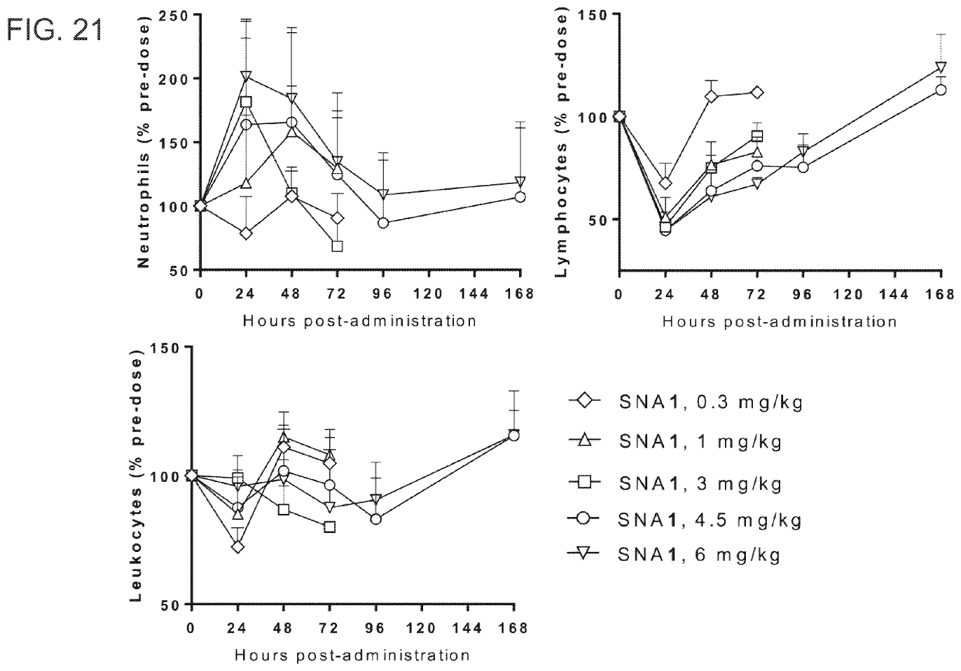

As the expression of TLR9 is different in rodents and primates (26-29), immune response profiles of SNA1 in vivo in NHPs were evaluated. Subcutaneous administration of SNA1 in cynomolgus monkeys induced dose-dependent increases in both B cell and pDC activation, and pDC maturation at 24 hr post SNA administration (FIG. 20A). SNA1 also showed activation of NK cells, T cells, and mDCs at the same time point (FIG. 20A). SNA1 administration led to a dose-dependent serum cytokine induction (FIG. 20B). However, the peak concentrations varied from 8 to 16 hr depending on the cytokine type and also the dose of SNA administered (FIG. 20C). In NHPs, a T_{H}1-type cytokine profile was observed as seen in in vitro mouse and human primary cell cultures and in vivo mouse studies. In addition, transient changes in the levels of circulating blood cell populations were observed at all dose levels studied. Circulating blood cell populations returned to pre-dose levels within 72-96 hr following SNA administration (FIG. 21) as has been reported with other TLR9 agonissts in primates (30, 31).

### Tumor Immunotherapy With TLR9 Agonist SNAs

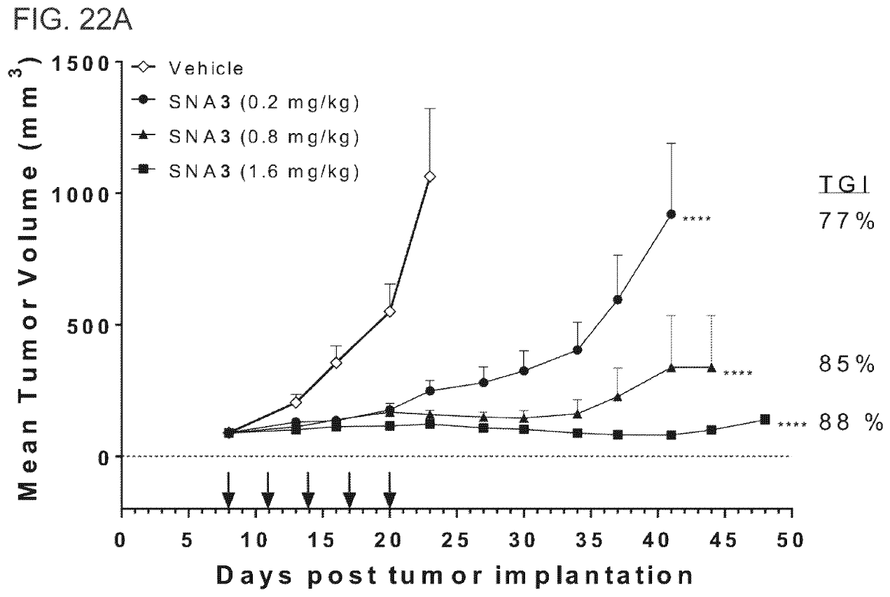

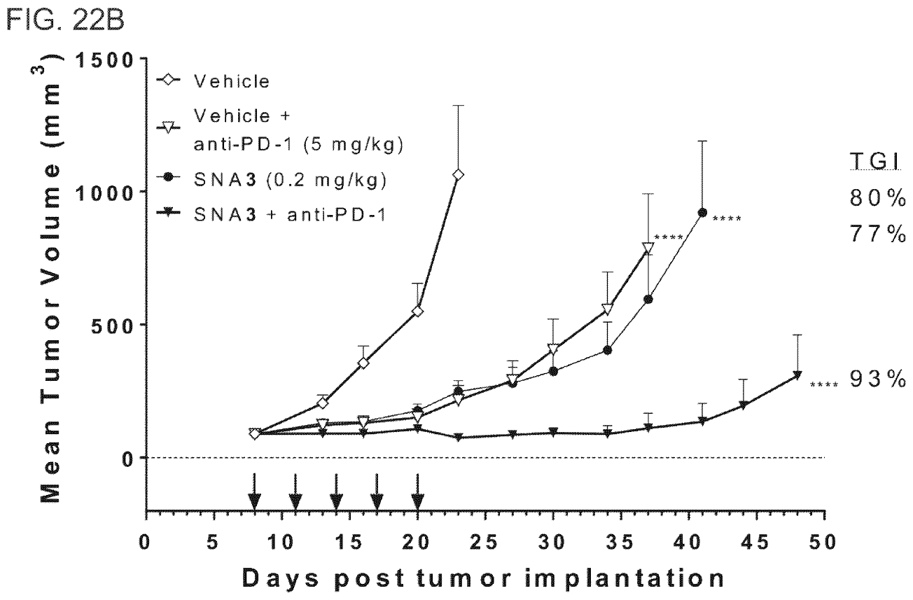

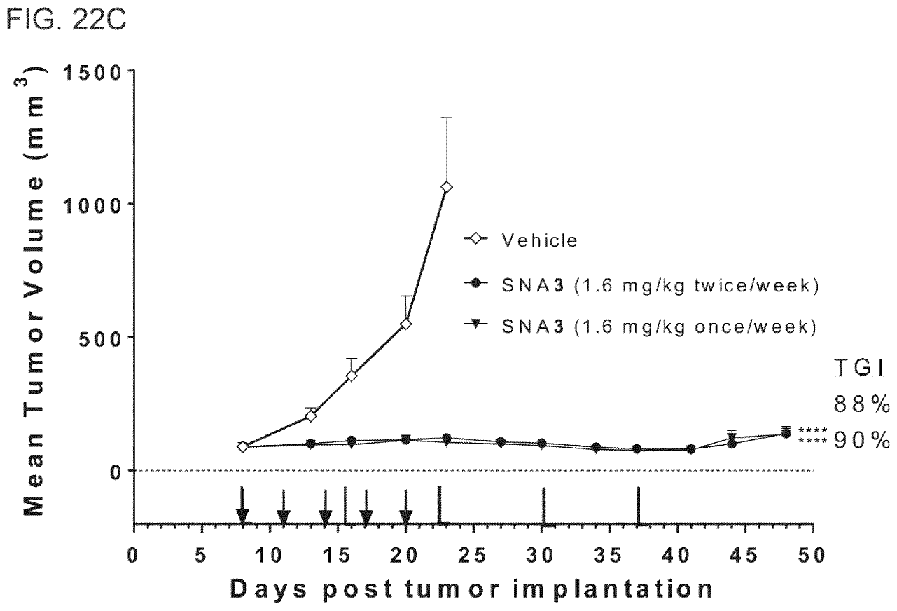

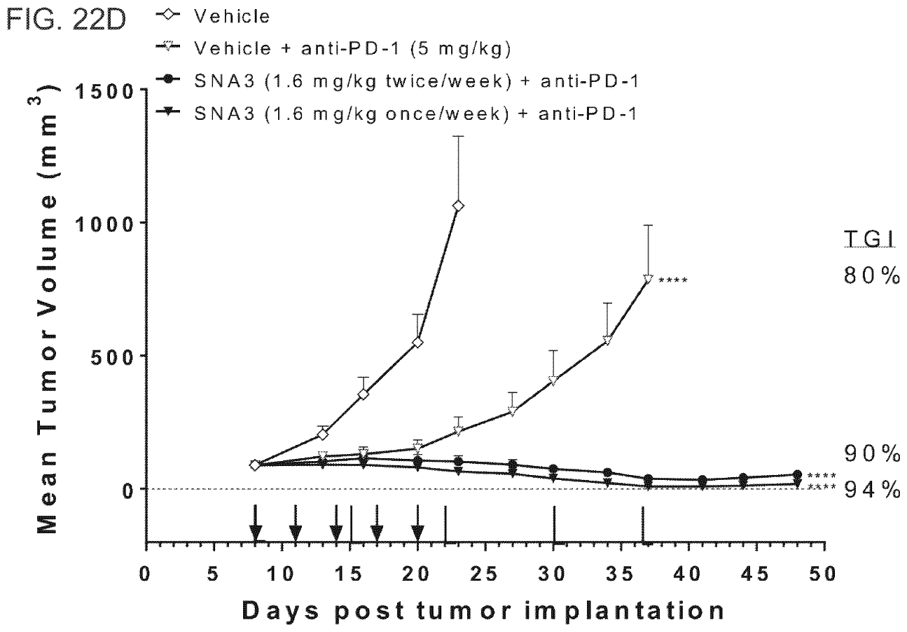

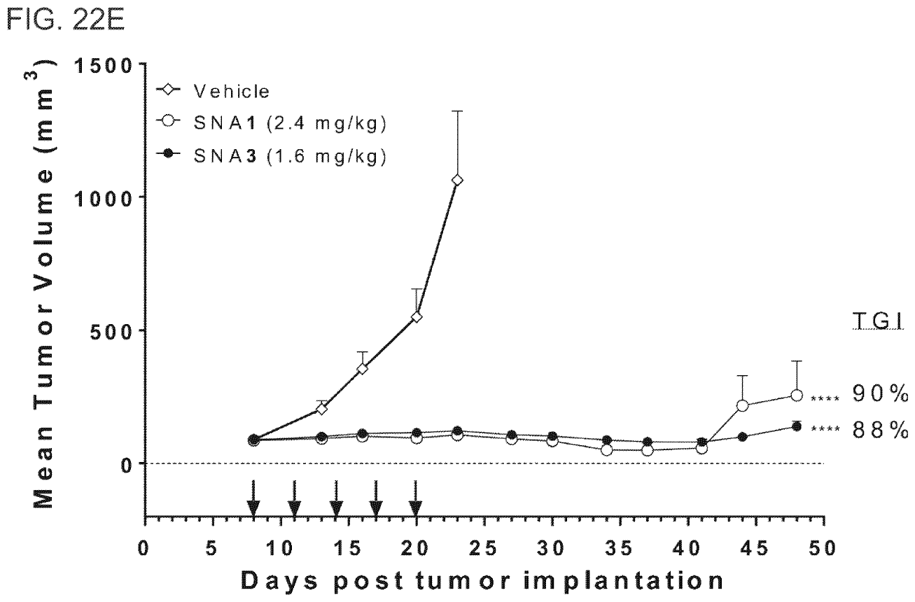

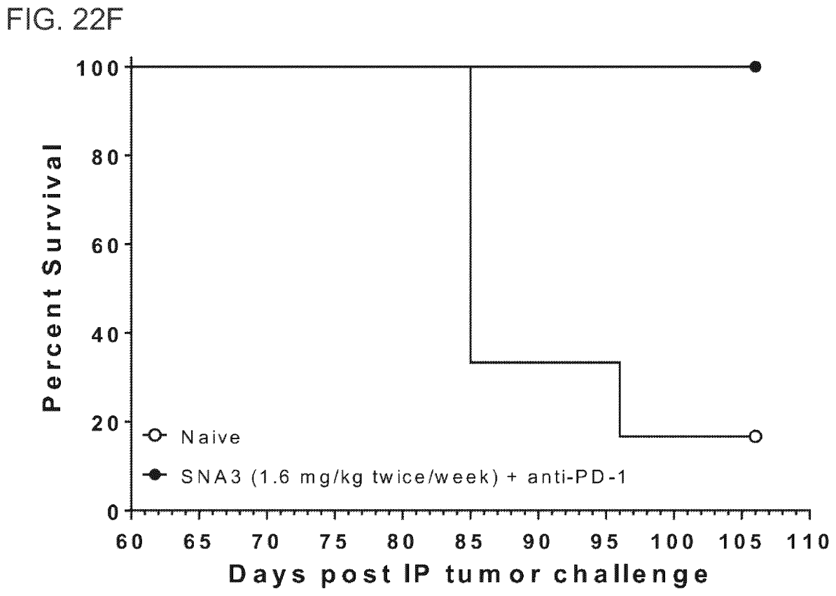

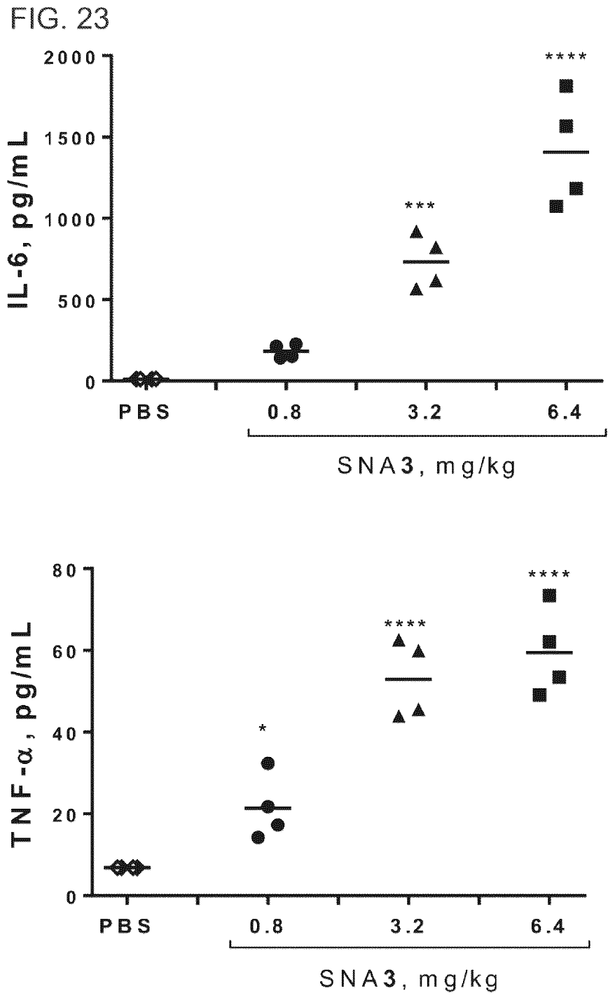

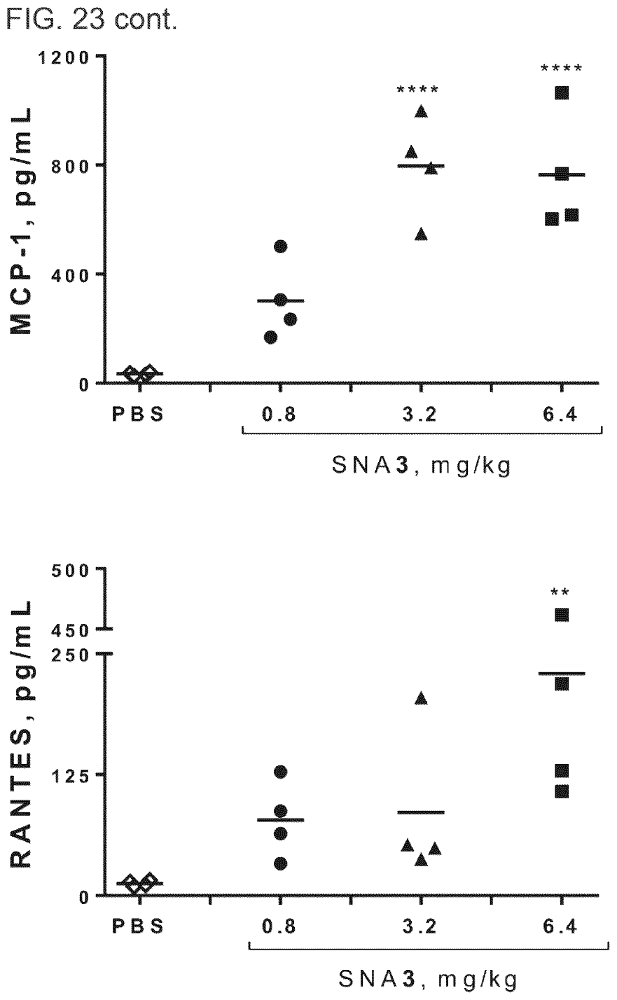

Having seen strong, sustained T_{H}1-type cytokine induction by SNA1 and SNA3 in vivo in NHPs and rodents, respectively, the efficacy of SNA3 in murine tumor models was assessed. Mice bearing MC38 colorectal tumors were injected intratumorally with 0.2, 0.8, and 1.6 mg/kg of SNA3 twice weekly for a total of five times beginning when the mean tumor volume (MTV) reached about 100 mm³. There was a statistically significant dose-dependent tumor growth inhibition (TGI) at all three dose levels (FIG. 22A). At the highest dose, an 88% TGI was observed. Concomitant with TGI, a dose-dependent increase in mouse survival was observed in SNA3-treated groups compared with mice in vehicle group. Median survival was about 40 days in the lowest dose group and >50 days in the two higher dose groups compared with 23 days for the vehicle group. These results demonstrate that TLR9 agonist SNA shows potent TGI and prolongs mice survival. To assess innate immune cytokine induction by SNA following intratumoral administration, the serum cytokine response to SNA3 in tumor bearing mice at 4 hours following the first dose administration in a separate study was measured. A dose-dependent T_{H}1-type cytokine induction in the serum of tumor bearing mice was observed (FIG. 23).

### Tumor Immunotherapy With TLR9 Agonist SNAs in Combination With Anti-PD-1

Tumor therapy has benefited greatly in recent years due to the availability of CPIs (32), which function by reducing inhibition of immune responses, thereby allowing expansion of anti-tumor immune responses. Unfortunately, CPIs are only effective in 10-30% of patients (33, 34), so there is a strong need for combination therapies to enhance CPI efficacy. Combination of an immunostimulatory TLR9 agonist SNA, which promotes immune responses, with CPIs, which support expansion of immune responses, is a rational approach to synergize the mechanisms of these two therapeutic approaches. The combination of SNA3 at 0.2 mg/kg dose intratumorally and an anti-PD-1 antibody at 5 mg/kg dose intraperitoneally were administered in the MC38 colorectal tumor model. Both agents were administered twice a week for a total of five times starting when the MTV was about 100 mm³. Synergy of SNA and anti-PD-1 combination treatment was observed, with up to 93% TGI compared with 77% and 80% TGI for SNA3 and anti-PD-1 monotherapies, respectively (FIG. 22B). Median survival of mice in the combination treatment was >50 days compared with about 40 days in both monotherapy groups or about 23 days in vehicle group.

In the above experiments SNA3 was administered twice a week for five times. Next, the impact of SNA dosing schedule on tumor growth was assessed by administering 1.6 mg/kg dose of SNA3 once or twice a week for five times either as a monotherapy or in combination with anti-PD-1 in the mouse MC38 colorectal tumor model. Once weekly dosing schedule of SNA monotherapy showed similar TGI and mice survival as that of twice weekly treatment groups (FIG. 22C). Once weekly and twice weekly dosing of SNA+anti-PD-1 combination therapy were also assessed. Since 88-90% TGI was achieved with 1.6 mg/kg SNA monotherapy, there were only small additional gains to 90-94% TGI in the combination therapy groups. As seen with SNA monotherapy, once weekly dosing of SNA+anti-PD-1 combination therapy showed similar TGI to twice weekly dosing of SNA+anti-PD-1 combination therapy (FIG. 22D).

Since human TLR9 agonists are known to engage mouse TLR9, antitumor effects of human and mouse selective SNAs 1 and 3, respectively, in the MC38 tumor model were compared. The dose levels for this study were selected based on serum cytokine dose response studies for the two compounds in mice (FIG. 17D and FIG. 19B). Based on these studies, a 50% higher dose was anticipated to be appropriate for SNA1 compared with SNA3. The MC38 tumor model study was carried out at a dose of 2.4 mg/kg and 1.6 mg/kg of human (SNA1) and mouse (SNA3) selective SNAs, respectively. As expected, SNA1 and SNA3 produced similar TGI and mouse survival to one another as monotherapies (FIG. 22E) and in combination with anti-PD-1. These results demonstrate the anti-tumor efficacy of SNA1 as well as the utility of the SNA structure with different oligonucleotide sequences.

As mice in several treatment groups survived through the end of the study (day 50), the surviving mice were rechallenged in the twice weekly SNA3 (1.6 mg/kg)+anti-PD-1 treatment group (see FIG. 22D) with MC38 tumor cells intraperitoneally. As a control, a group of naïve mice were challenged in an identical manner. All naïve mice in the control group showed tumor growth and 5 of 6 were sacrificed due to tumor burden within 39 days from the day of tumor inoculation, whereas no mouse showed tumor growth in the previously treated group, suggesting a strong tumor-specific memory response in the treated group (FIG. 22F).

### SNA Monotherapy in the EMT6 Breast Cancer Model

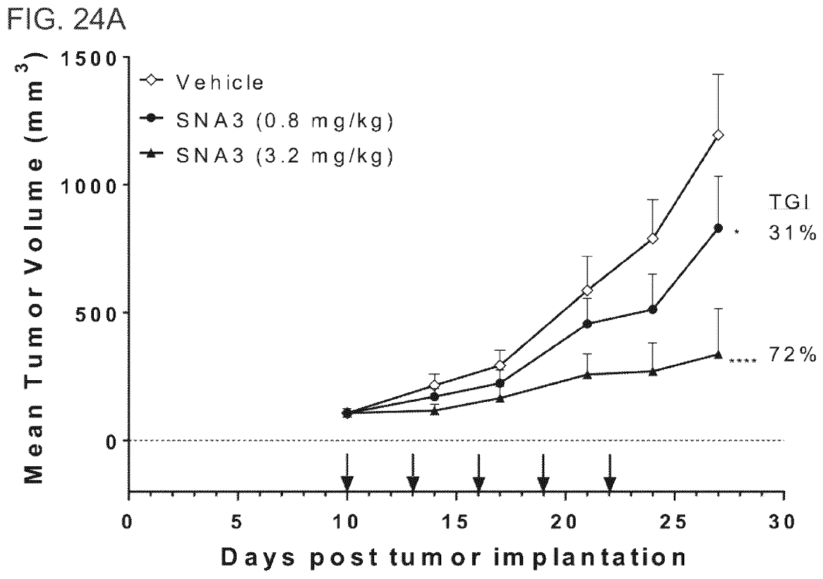

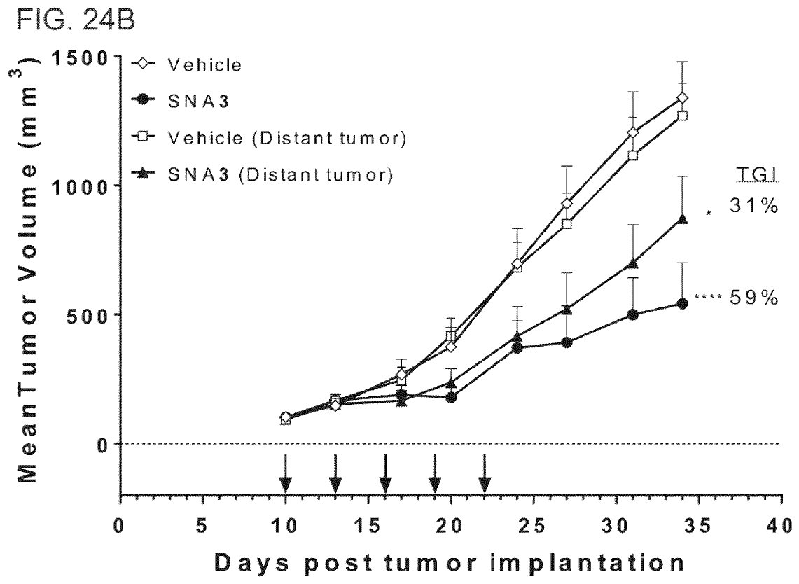

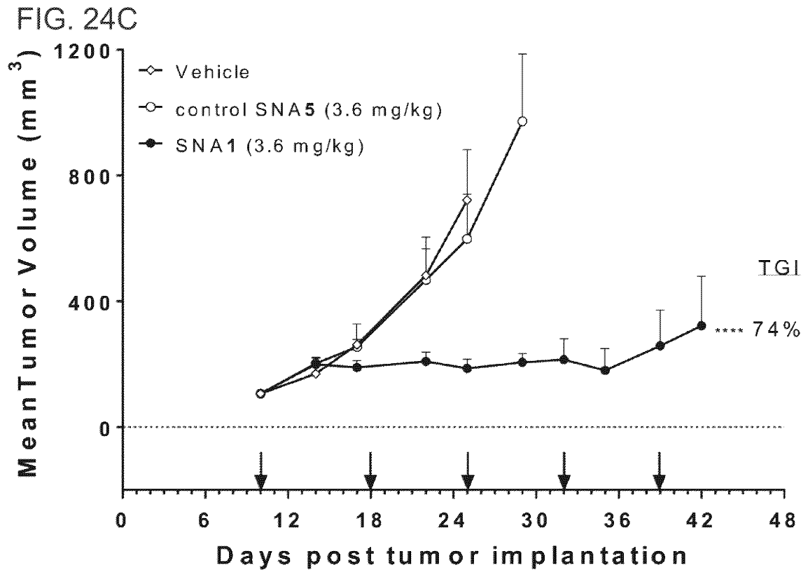

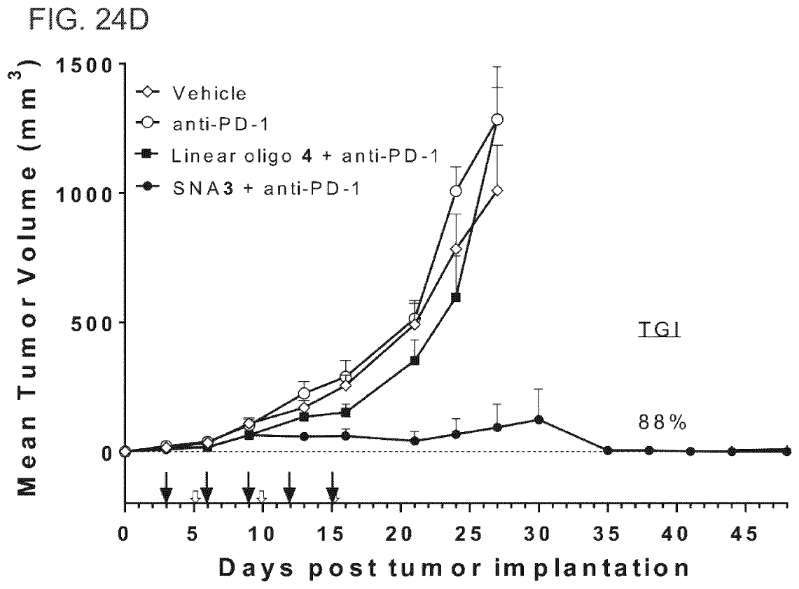

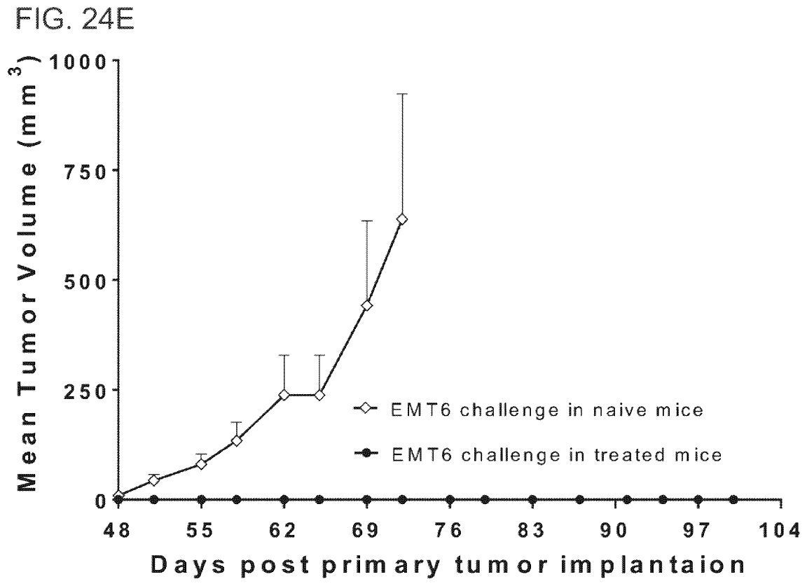

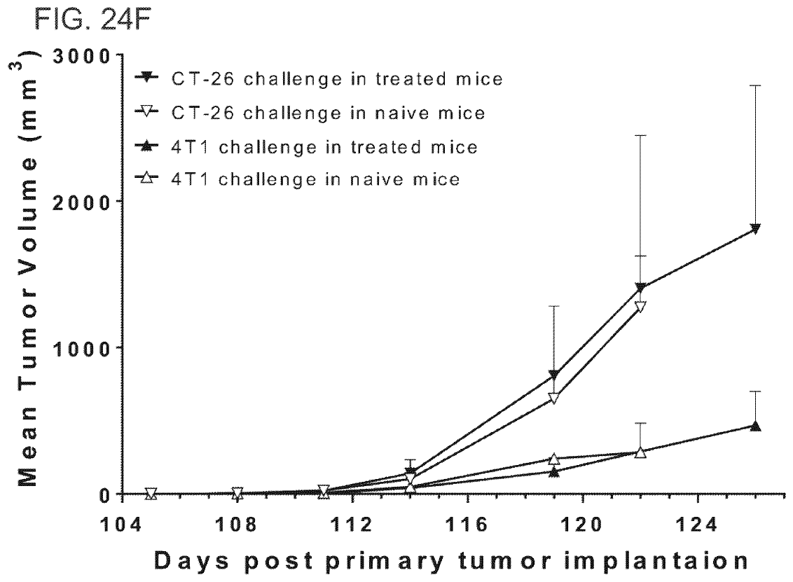

The efficacy of TLR9 agonist SNAs was next assessed in a tumor model that is insensitive to anti-PD-1 antibody treatment (34), the murine EMT6 breast cancer model. Mice were inoculated with EMT6 tumor cells on day 0. Beginning 10 days after tumor inoculation when the MTV was 100 mm³, SNA3 was administered at 0.8 and 3.2 mg/kg doses subcutaneously every three days for a total of 5 times. As in the MC38 model, in the EMT6 breast cancer model also SNA treatment resulted in dose-dependent statistically significant TGI (FIG. 24A). Further, inhibition of tumor growth by SNA3 resulted in prolonged survival of mice. The mice in vehicle group showed a median survival of 33.5 days and the median survival of mice in 0.8 and 3.2 mg/kg SNA3 dose groups was 39 and >50 days, respectively.

The anti-tumor effect of SNA therapy was then assessed on contralateral tumors. Mice were inoculated with EMT6 tumors on both flanks on day 0 and treatment began on day 10 when MTV reached 100 mm³. SNA3 was administered at 3.2 mg/kg peritumorally by subcutaneous injection near the tumor on one flank, and the tumor growth of the tumors on both flanks was monitored. Treatment with SNA3 monotherapy resulted in significant TGI of the tumors on both flanks (FIG. 24B).

Additionally, the efficacy of human-specific SNA1 and negative control SNA5 in the EMT6 model was studied. Mice bearing 100 mm³ EMT6 tumors were injected intratumorally with SNA1 or control SNA5 at 3.6 mg/kg once weekly for 5 weeks. As seen with SNA3, mice treated with SNA1 monotherapy (FIG. 24C) exhibited statistically significant TGI that was not observed with control SNA5. SNA1 monotherapy also resulted in a concomitant increase in survival of tumor-bearing mice with all mice surviving >42 days, whereas median survival of the mice treated with vehicle and control SNA5 were 31.5 and 35.5 days, respectively.

### Combination Therapy With Anti-PD-1 in EMT6 Model

The effect of SNA3 and anti-PD-1 combination in the EMT6 tumor model was next studied. SNA3 or linear oligo 4 was administered subcutaneously at a dose of 0.8 mg/kg every three days for a total of five times starting on day 3 following tumor inoculation. Anti-PD-1 was administered either alone or in combination with SNA3 or linear oligo 4intraperitoneally at a dose of 10 mg/kg every five days for a total of three times starting on day 5 following tumor inoculation. Anti-PD-1 alone did not show TGI compared with vehicle (FIG. 24D). Previous reports have shown that the EMT6 tumor was resistant to anti-PD-1 treatment and the present observations are consistent with these studies (35). Linear oligo 4 combined with anti-PD-1 had minimal impact on TGI. Whereas, SNA3 combined with anti-PD-1 resulted in complete regression of the tumor in 7 of 8 mice (FIG. 24D) and the mice survived >44 days.

On day 44, the surviving mice in SNA3+anti-PD-1 treatment group were re-challenged with EMT6 tumor cells in the opposite flank along with a group of naïve mice as control. Naïve mice in the control group developed tumors as expected. By contrast, the mice previously treated with SNA3+anti-PD-1 did not show tumor growth and survived up to day 104 (FIG. 24E), indicating that a tumor-specific adaptive memory response had been established in these mice following SNA3+anti-PD-1 treatment. On day 104, the surviving mice were challenged with heterologous tumor cells, either CT26 colorectal or 4T1 breast tumor cells. These heterologous tumors grew as in the case of naïve control mice (FIG. 24F), indicating that the SNA+anti-PD-1 treatment led to tumor-specific adaptive immune responses against EMT6 tumors, but not the heterologous CT26 and 4T1 tumors.

### SNA Treatment of Tumor-Bearing Mice Alters Regulatory and Effector T-Cell Responses

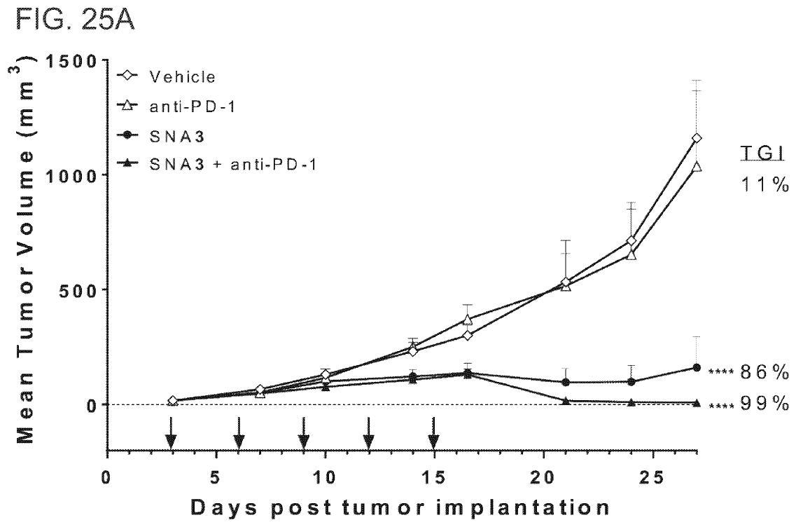

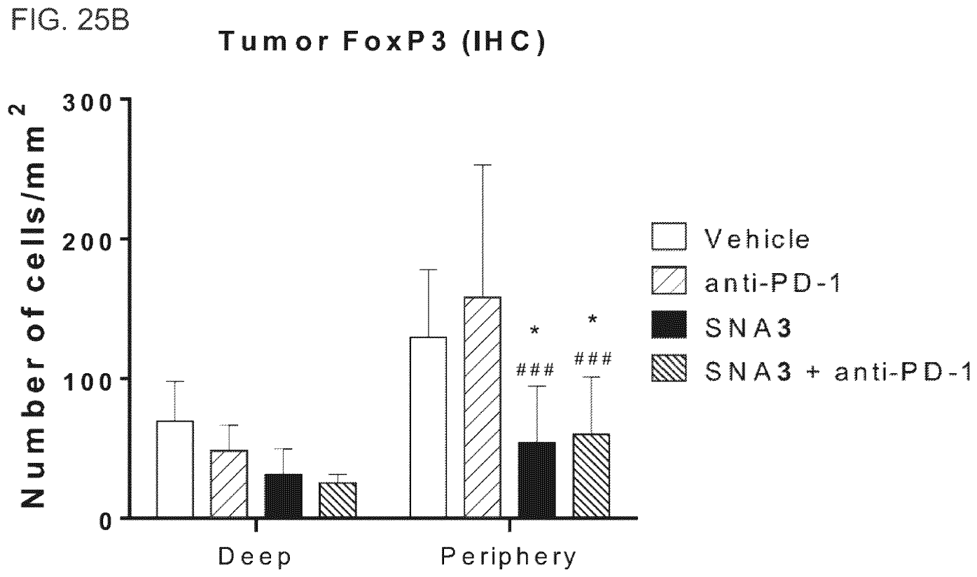

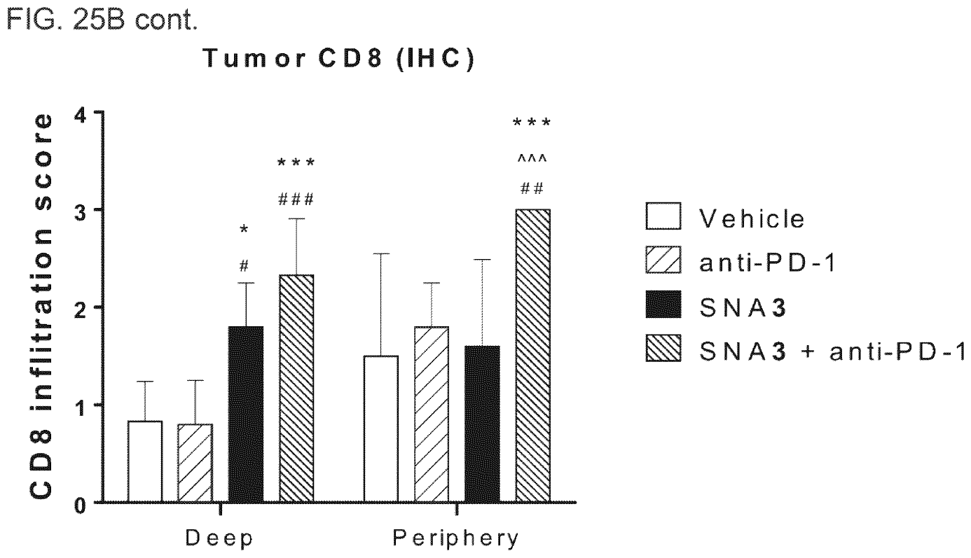

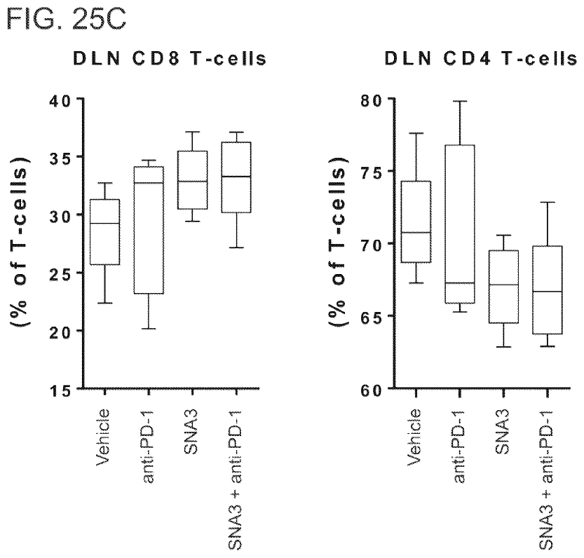

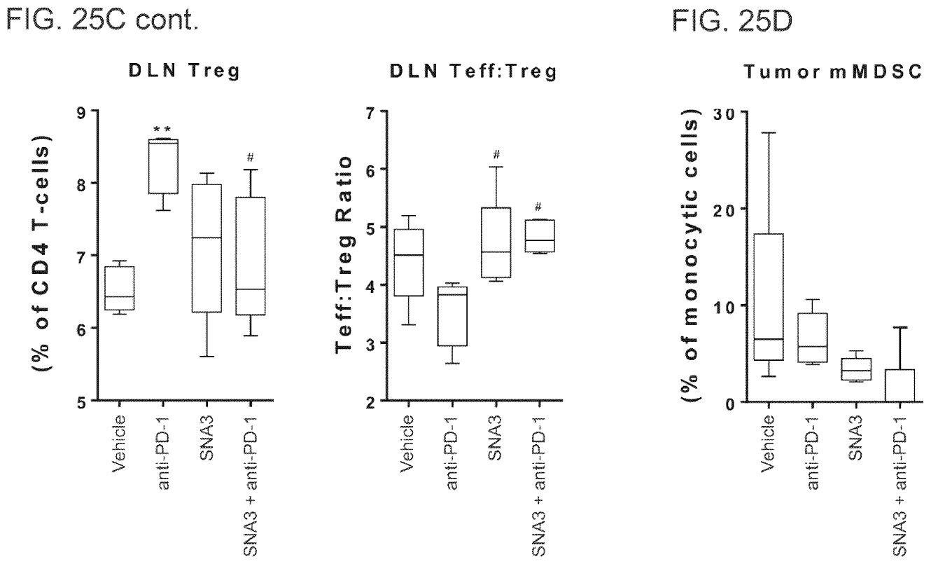

To understand the mechanism behind the anti-tumor immunity induced by SNA and the combination of SNA and anti-PD-1, the T cell responses in TME and in the DLN in the EMT6 tumor model were examined. On day 10 following tumor inoculation (one day following the third dose of SNA), mice bearing EMT6 tumors were sacrificed for immunological assessment (FIGS. 25A-25D). FoxP3 regulatory T cells (Treg) and CD8 effector T cells (Teff) were measured in the tumors by immunohistochemistry and in the DLN by flow cytometry. It was observed that SNA monotherapy, which showed TGI (FIG. 25A), decreased Treg in the peripheral tumor and increased Teff in the deep tumor (FIG. 25B), and increased the Teff:Treg ratio in the DLN (FIG. 25C). Anti-PD-1 monotherapy, which was ineffective at inhibiting EMT6 tumor growth, induced no changes in T cell levels in TME, but led to an increase in Treg cells and a decrease in Teff:Treg ratio in the DLN. The combination of SNA with anti-PD-1, which exhibited the strongest TGI, reduced peripheral tumor Treg, increased peripheral and deep tumor Teff, prevented or reversed the increased DLN Treg that is induced by anti-PD-1 alone, and increased the DLN Teff:Treg ratio. These data suggest a clear correlation between Teff and Treg levels and inhibition of tumor growth in combination therapy with SNA and anti-PD-1. In addition, the level of mMDSC in these tumors was examined. Trends toward reduced mMDSC in tumors following SNA monotherapy and further reduction following combination therapy with SNA and anti-PD-1 were observed (FIG. 25D).

### Intravenous Administration of SNA in the EMT6 Tumor Bearing Mice

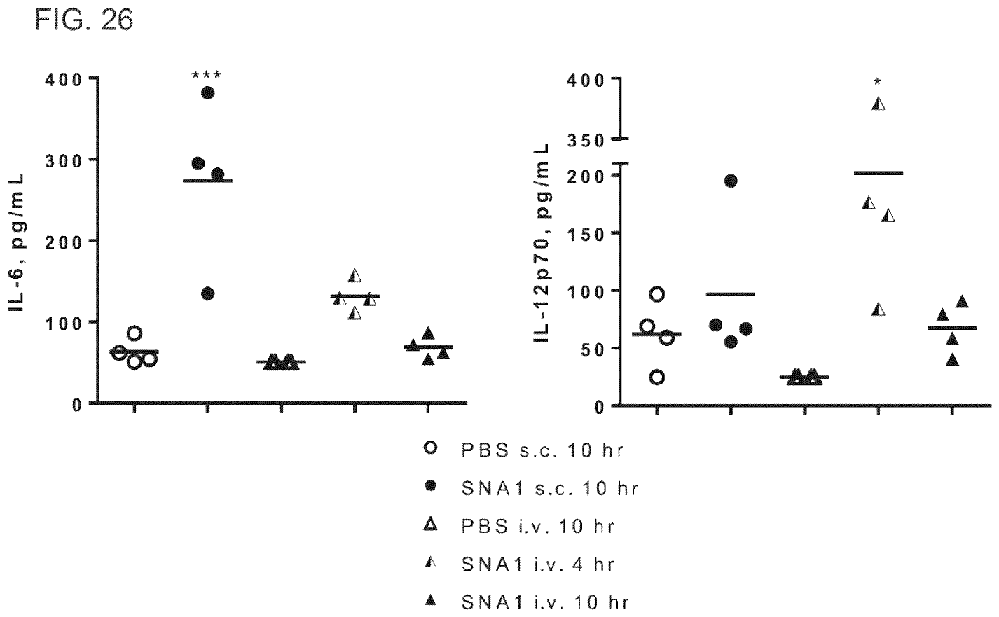

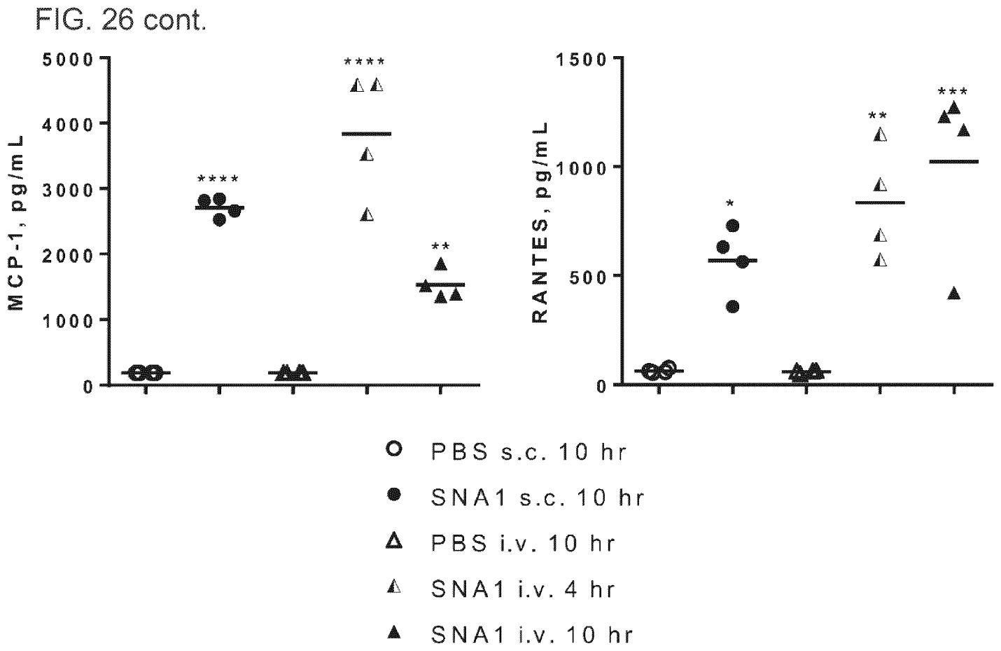

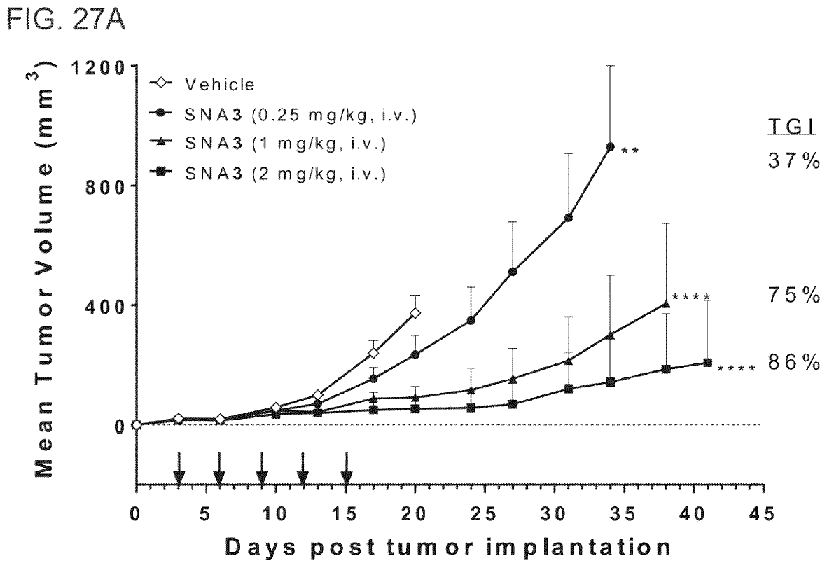

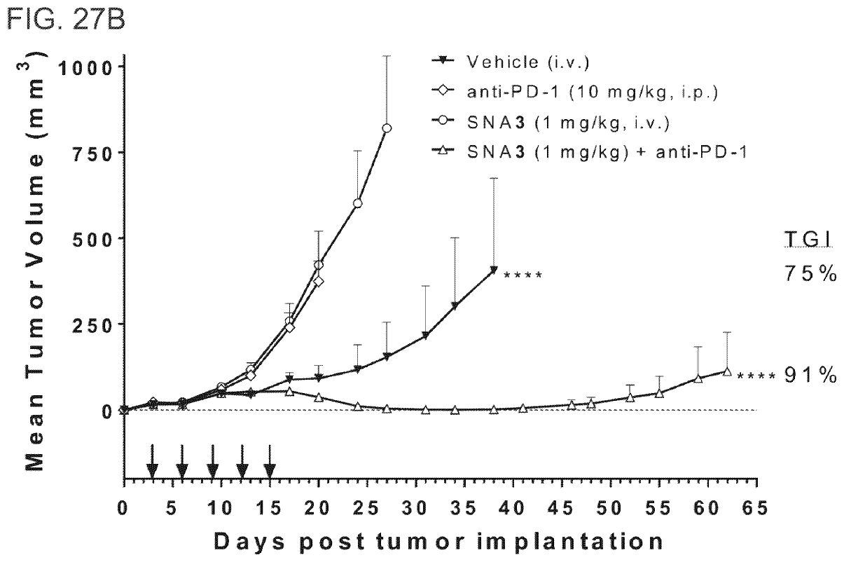

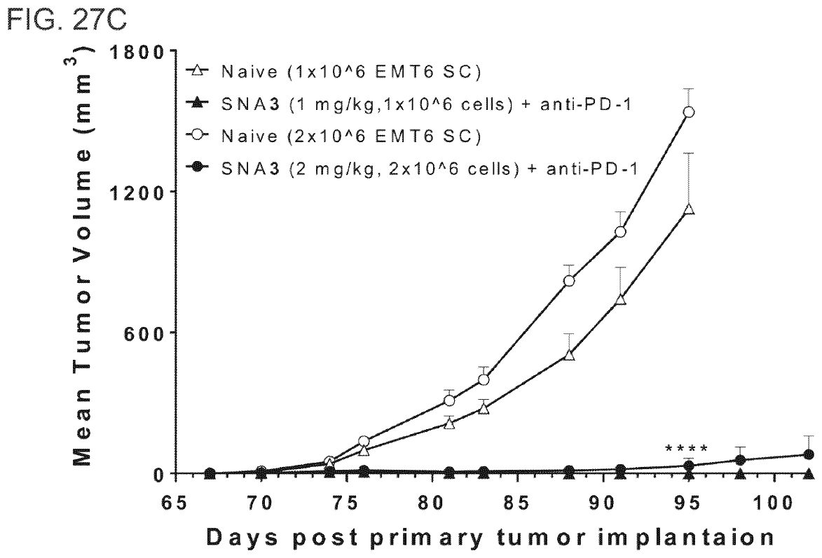

Intravenous dosing of the TLR9 agonist CpG 7909 in healthy volunteers did not induce a cytokine response (31). As an initial step, serum cytokine induction in mice treated with SNA subcutaneously or intravenously was compared. Similar cytokine profiles were observed, although the cytokine response occurs earlier (4 vs. 10 hr) when the SNA1 was administered intravenously (FIG. 26). Then it was asked whether TLR9 agonist SNA would show antitumor effects when administered intravenously in mice bearing EMT6 tumors. Intravenous administration of SNA3 (0.25, 1, or 2 mg/kg) either alone (FIG. 27A) or in combination with intraperitoneally administered anti-PD-1 led to a dose-dependent TGI (FIG. 27B). In addition, mouse survival increased concomitantly with TGI. Mice in both vehicle and anti-PD-1 monotherapy groups showed similar median survivals of 34 days. At 0.25 mg/kg SNA3, the median survival was 42 days as a monotherapy and 58.5 days when combined with anti-PD-1. At 1 and 2 mg/kg dose levels, the median survival was >63 days both as a monotherapy and in combination with anti-PD-1. These results demonstrate that TLR9 agonist SNAs are effective following IV administration either as monotherapy or in combination with anti-PD-1.

To further evaluate if intravenous administration of SNA would also lead to tumor-specific long-term memory responses, surviving mice from anti-PD-1 combination therapy groups of SNA3 (1 and 2 mg/kg groups) were subsequently challenged with 1× or 2× EMT6 tumor cells, respectively. Regardless of the tumor cell number used for rechallenge, the tumor was rejected and showed no tumor growth (FIG. 27C).

### Discussion

TLR9 agonists have been shown to promote innate and adaptive immune responses, including B cell proliferation, Ig production, T_{H}1-type cytokine induction, and surface marker activation. Based on the specific immune response profiles induced by different classes of TLR9 agonists, they have been extensively evaluated in preclinical and clinical studies as treatments for cancers, asthma and allergies, infectious diseases, and as vaccine adjuvants (13). The B-class TLR9 agonists, CpG 7909, ISS 1018, IMO-2055, and MGN1703 have been evaluated as potential cancer therapy in clinical trials as monotherapy and in combination with peptides, monoclonal antibodies, radiotherapy, and chemotherapy (13, 36-38). However, no clinical benefit was observed either as monotherapy or in combination with anticancer agents underscoring the need for more potent TLR9 agonists.

SNAs are a novel class of agents in which oligonucleotides are densely packed on a nanoparticle leading to a three-dimensional arrangement of oligonucleotides compared with linear oligonucleotides. The SNAs have been shown to facilitate increased cellular uptake and resist nuclease degradation (17, 39). Therefore, known TLR9 agonists have been selected, such as linear oligo 2 and 4 that have been extensively studied in tumor models and/or clinical trials and create SNA structures (SNA1 and SNA3, respectively) to establish broad therapeutic utility of SNAs in immuno-oncology applications.

The current studies clearly demonstrated that an oligonucleotide presented in an SNA format (SNA1) is more efficiently taken up by immune cells in systemic circulation than the same oligonucleotide that is not in SNA format (linear oligo). These results are consistent with earlier observations of greater uptake of SNA into RAW 264.7 cells (17) and show efficient uptake of SNAs by primary cells. Moreover, SNA1 stimulated TLR9 selectively and more potently than the linear oligonucleotide in cell lines. The increased TLR9 activation can be ascribed to increased i) cellular uptake and ii) nuclease stability of oligonucleotides in SNA format compared to linear oligonucleotides. SNAs have been shown to exhibit greater nuclease stability than linear oligonucleotides as a result of increased negative charge density and salt gradient around the nanoparticle structure leading to decreased accessibility and activity of nucleases to oligonucleotides in SNA (39).

In primary mouse splenocytes and human PBMCs, SNA3 and SNA1, respectively, induce T_{H}1, but not T_{H}2,-type cytokine secretion. The cytokine induction is time and SNA dose dependent. In both rodent and human primary cells, SNAs induce relatively higher levels of cytokines compared with the linear oligonucleotide. No or background levels of cytokine secretion is observed with control SNA in which CpG dinucleotides are replaced with GpC dinucleotides. These results establish that the CpG oligonucleotides in SNA format selectively interact with TLR9 and induce TLR9-mediated immune responses more efficiently than linear CpG oligonucleotides.

Beyond in vitro studies, it has beendemonstrated that the SNAs induce TLR9-mediated immune responses in vivo in mice and in NHPs. A single dose of SNA in mice lead to T_{H}1-type systemic cytokine induction (40) and these results are consistent with the in vitro studies as well. In addition, SNAs show slower and more durable cytokine induction profiles in mice compared with linear oligonucleotide of the same sequence. Linear CpG oligonucleotides have been shown to induce peak levels of cytokines within 4-8 hr post administration which return to pre-dose levels by 12-16 hr depending on the type of cytokine induced (23-25). By contrast, SNAs have shown a slower kinetics of cytokine induction with peak levels at 10-16 hr post administration which return to pre-dose levels by 20-24 hr or sometimes longer than 24 hr, depending on the cytokine type. It is hypothesized that the slower kinetics of cytokine induction by SNAs could be a result of the nanoparticle structure that leads to slower passage through lymphatics to draining lymph nodes compared with linear oligonucleotides. In addition to subcutaneous route of administration, intramuscular, intravenous, and nasal routes of administration have been studied and similar T_{H}1-type cytokine profiles in mice have been observed.

TLR9 is expressed more widely in rodents (B cells, pDCs, macrophages, monocytes, and mDCs) than in primates (B cells and pDCs) (27, 29). As a proof of concept, the present studies demonstrate that acute administration of SNAs in cynomolgus monkeys induced dose-dependent immune responses without any adverse events. The SNA doses administered in NHP were well tolerated without significant local injection site reactions and changes in clinical parameters (monitored clinical observations are listed in Materials and Methods section). SNA administration led to activation of NK cells, B cells, T cells, mDCs, and pDCs and maturation of pDC populations in the circulation within 24 hr of treatment. The immune cell activation was dose-dependent and peaked at 4.5 mg/kg dose, and then blunted at the highest dose of 6 mg/kg. These results are consistent with previous reports that TLR9 agonists produce bell shaped dose response curves (8, 41, 42) as the immune regulatory circuits are activated following threshold level of inflammatory response induction (10, 43). IP-10 has been shown to be the most reliable biomarker for TLR9 activation in primates (44). Consistent with this observation, SNA induced rapid and robust dose-dependent IP-10 induction in NHPs. SNA administration to NHPs also results in transient hematological changes in systemic circulation as determined by lymphocyte, leukocyte, monocyte, and eosinophil decreases and neutrophil increase within 24 hr. As expected, these hematological changes return to pre-dose levels in the next day or two. These hematological changes in the peripheral blood are also consistent with reported results for other TLR9 agonists in primates and for recombinant cytokines in humans (45-47). These results demonstrate that SNAs engage TLR9 and induce potent TLR9-mediated immune responses without any adverse events in rodents and NHPs.

In murine tumor models, administration of TLR9 agonist SNAs induce dose-dependent reductions in tumor growth and increase in survival in the MC38 colorectal and EMT6 breast cancer models. Both murine-and human-specific SNAs are active in mice, but as expected the mouse-specific SNA is active at lower dose levels.

CPI are a class of therapeutics that function by blocking certain immune-inhibitory proteins, allowing anti-tumor immune responses to develop or expand. CPI therapy has flourished in recent years with FDA-approved drugs targeting CTLA-4, PD-L1 and PD-1 checkpoint proteins, and other targets are under development. Yet a considerable number of patients relapse following treatment or do not respond to CPI treatment at all (33, 34). Several studies have shown that the tumor escape from CPI treatment could be a result of exhausted effector T cells, functionally impaired antigen-presenting cells (APCs), and/or infiltration of tumor supporting cell types such as MDSCs (48). TLR9 agonists are known to produce rapid innate as well as long-term adaptive immune responses. TLR9 agonists have been shown to produce a broad activation of immune cells including APCs and CD4 and CD8 T cells, and suppress Treg and MDSCs in TME (49-51). Therefore, the use of TLR9 agonist SNA could be a rational approach to combine with CPI to effectively treat a larger patient population. Consistent with the expected mechanism, SNAs show synergy with anti-PD-1 in both anti-PD-1 sensitive (MC38) and insensitive (EMT6) tumor models with increased TGI and mice survival.

The administration of SNA to tumor bearing mice show rapid dose-dependent innate immune responses as determined by cytokine induction in serum, which are required for bridging the adaptive immune responses in the presence of tumor-associated antigens released from the dying tumors. Mice bearing MC38 or EMT6 tumors that are treated with TLR9 agonist SNAs are not susceptible to re-challenge with the same tumor cells, indicating that SNA treatment induces the formation of immunological memory against the treated tumor cells. However, challenge with heterologous tumor cell lines CT-26 or 4T1 results in tumor growth, confirming that the immunological memory response is tumor-specific.

Mechanistically, in the anti-PD-1 insensitive EMT6 tumor model, SNA treatment led to an increased ratio of T-effector cells to T-regulatory cells, both in the TME and DLN. Although anti-PD-1 monotherapy increased T-regulatory cells, this effect is overcome in the combination treatment with TLR9 agonist SNA. Further, a reduction in Tregs and mMDSCs in TME/DLN following SNA treatment could support the increased antitumor effectiveness observed in the combination treatment groups. The anti-tumor activity of SNA following intratumoral, subcutaneous, or intravenous routes of administration is evident from the current tumor model studies. These studies demonstrate the nanoparticle-based SNAs can be utilized by a variety of routes of administration in humans.

Taken together, the current results demonstrate that TLR9 agonist SNAs are taken up by primary immune cells and activate TLR9 to a greater extent than a TLR9 agonist of the same sequence that is not in SNA format (linear oligo) in vitro and in vivo in mice and non-human primates. TLR9 agonist SNA shows dose-dependent tumor growth inhibition and prolongs survival of tumor bearing mice as monotherapy and enhances anti-PD-1 effectiveness in combination treatment following subcutaneous, intratumoral, and intravenous routes of administration. The mode of action of TLR9 agonist SNAs either alone or in combination with CPI is through rapid innate immune responses followed by induction of tumor-specific adaptive immune responses, increased infiltration of lymphocytes, increased effector cell population, and decreased Tregs as well as mMDSCs in TME and/or DLN. In contrast to the failures of linear TLR9 agonists for cancer immunotherapy in the past, the studies reported here strongly support the use of TLR9 agonist SNA as a potential candidate for the treatment of cancers as a monotherapy and in combination with CPI.

### Materials and Methods

### DNA Synthesis and Purification

Cholesterol-conjugated CpG and GpC oligonucleotides were used for SNA synthesis. Cholesterol-CpG and GpC oligonucleotides were synthesized in 5′-to 3′-direction and the linear CpG oligonucleotides were synthesized in 3′-to 5′-direction using ≢-cyanoethyl2phosphoramidite chemistry on appropriate solid supports. Syntheses were carried out on 0.2to 2.2 mmole scale on ÄKTA oligopilot plus 100 synthesizer (GE Healthcare). The required 3′-and 5′-phosphoramidites of dA, dC, dG, T, spacer-18 (hexaethyleneglycol), and TEG-cholesterol were obtained from ChemGenes Corporation (Wilmington, MA). Phenylacetyl disulfide (PADS) was used as an oxidizing agent to obtain phosphorothioate backbone. After the synthesis, oligonucleotides were cleaved from the solid support and deprotected by standard protocols using ammonia solution, purified by RP-HPLC, and concentrations were measured using the UV absorbance at 260 nm (Cary 100 Bio UV-Visible Spectrophotometer). All the oligonucleotides synthesized were characterized by MALDI-TOF mass spectrometry (Brucker Autoflex III) for molecular mass and AE-HPLC for purity. The purity of the oligonucleotides used in the studies ranged from 90% to 98% (see Table 4 for oligonucleotide characterization data). Oligonucleotides with fluorescein label on the 3′-terminal T were synthesized using the protocols described above. The compounds were tested for endotoxin by the Kinetic Turbidimetric assay and the levels of endotoxin were <1 endotoxin unit/mg.

TABLE 4
Analytical data of oligonucleotides and SNAs used in the study. Compound #1
corresponds to SEQ ID NO: 4, Compound #2 corresponds to SEQ ID NO: 5, Compound #3
corresponds to SEQ ID NO: 6, Compoud #4 corresponds to SEQ ID NO: 7, and Compound
#5 corresponds to SEQ ID NO: 8.

| | | Oligonucleotide | | | Num |
|---|---|---|---|---|---|
| Compound | | Mass | Ob- | SNA | Mean |
| # | Sequence (5′→3′)* | Calculated | served | PDI | (nm) |
| 1 | TCGTCGTTTTGTCGTTTTGTCGTT-(SP18)₂-TEG- | 9143 | 9140 | 0.201 | 27.7 |
| | cholesterol | | | | |
| 2 | TCGTCGTTTTGTCGTTTTGTCGTT | 7698 | 7694 | N/A | N/A |
| 3 | TCCATGACGTTCCTGACGTT-(SP18)₂-TEG- | 7809 | 7808 | 0.163 | 28.3 |
| | cholesterol | | | | |
| 4 | TCCATGACGTTCCTGACGTT | 6364 | 6365 | N/A | N/A |
| 5 | TGCTGCTTTTGTGCTTTTGTGCTT-(SP18)₂-TEG- | 9143 | 9140 | 0.211 | 25.9 |
| | cholesterol | | | | |

*All sequences contain a phosphorothioate backbone; SP18 stands for spacer-18 or hexaethyleneglycol linker; TEG stands for tetraethyleneglycol linker; underline indicates CpG. N/A-not applicable.

### SNA Synthesis

All steps to synthesize SNAs were performed in a sterile environment, and reagents used were endotoxin free. SNAs were synthesized by adding 30-fold molar excess of cholesterol-conjugated oligonucleotides to 21±2 nm DOPC liposomes in 1×PBS and incubated overnight at 4° C. to obtain about 30 oligonucleotides per liposome. SNA size was measured by DLS using a Zetasizer Nano ZS (Malvern Instruments, Malvern, UK).

### Fluorescently Labeled Oligonucleotide Synthesis and Uptake

Oligonucleotide synthesis was performed as described above, but with a fluorescein label on the 3′-terminal thymidine. SNA synthesis was performed as described above, but the 3′-cholesterol oligonucleotides with a fluorescein label on the 3′-terminal thymidine were loaded onto 50 nm DOPC liposomes at a ratio of 100 oligonucleotides per liposome.

### Reporter Cell Lines

HEK-Blue reporter cells (null1, hTLR3, hTLR7, hTLR8, hTLR9) were obtained from InvivoGen (San Diego, CA) and cultured according to the supplier's instructions. Cells were treated with TLR agonist SNAs, linear oligonucleotides, or control GpC SNAs as indicated in the text for 24 hours with no media change except where indicated otherwise; for shorter treatments of the agonist, the cell culture media was removed at the time points, cells were washed with complete media, and then fresh complete media was added. As positive controls, hTLR3-HEK-Blue cells were treated with 85 nM low molecular weight poly I:C (InvivoGen), hTLR7-HEK-Blue and hTLR8-HEK-Blue were treated with 1 μM R848, and null1-HEK-Blue were treated with 10 μg/mL PMA (InvivoGen). At 24 hours following addition of agonist, TLR activation was quantified using the QUANTI-Blue™ reporter assay (InvivoGen) according to the supplier's instructions.

### Primary Cell Isolation, Culture, and Cytokine Analysis

Primary mouse splenocytes were obtained from C57BL/6 mice. Primary human PBMC were processed using Ficoll (Ficoll-Paque® PREMIUM Medium (1.078 g/ml Density Max.); GE Healthcare) gradient density centrifugation method from buffy coat fractions obtained from healthy volunteers by Zen-Bio (Research Triangle Park, NC) and shipped overnight at ambient temperature. Both mouse splenocytes and hPBMC were used fresh (i.e. unfrozen). Primary cells were treated with TLR9 agonist compounds overnight. Cytokine levels were measured in cell culture supernatant using mouse or human multiplex cytokine arrays (Quansys, Logan, UT).

### Mouse Serum Cytokine Analysis

In vivo mouse serum cytokine studies were carried out at Avastus Preclinical Services (Cambridge, MA) according to the Avastus approved IACUC protocols. Female, 6-week old C57BL/6 mice were injected subcutaneously with TLR9 agonist compounds. At the indicated time, or at 10 hours if unspecified, whole blood was obtained and processed to obtain serum. Cytokine levels were measured in the mouse serum using mouse multiplex cytokine arrays as described above (Quansys).

Non-Human Primate studies

Non-human primate studies were performed at MPI Research (Mattawan, MI) according to MPI Research approved IACUC protocols. Each treatment group consisted of two male and two female cynomolgus monkeys, age 2-4 years, weighing 2-4 kg. Compounds were administered subcutaneously on day 1. Blood was drawn pre-dose and at the indicated time points for analysis by flow cytometry, hematology, and serum cytokine analysis. After collection of the final blood samples, animals were monitored for an additional ≥14 days prior to treatment with an additional dose or compound. Clinical monitoring of the study animals was performed at least twice daily and included, but was not limited to, evaluation of the skin, fur, eyes, ears, nose, oral cavity, thorax, abdomen, external genitalia, limbs and feet, respiratory and circulatory effects, autonomic effects such as salivation, nervous system effects including tremors, convulsions, reactivity to handling, and unusual behavior.

For hematology, blood was drawn pre-dose and at 24, 48, 72, and in some cases 96 and 168 hr post-dose. Hematological blood cell counts and differential was performed at MPI Research.

For flow cytometry, blood was drawn pre-dose and 24 hr post-dose and was used fresh. Flow cytometry was performed at FlowMetric (Doylestown, PA) using a BD FACS Aria instrument and assessed CD3+ T lymphocytes, CD3+ CD69+ activated T lymphocytes, CD3+ CD4+ helper T lymphocytes, CD3+ CD8+ cytotoxic T lymphocytes, CD3− CD16+ natural killer (NK) cells, CD3− CD16+ CD69+ activated natural killer (NK) cells, CD3− CD20+ B lymphocytes, CD3− CD20+ CD86+ activated B lymphocytes, CD3/8/14/20− HLADR+ CD11c− CD123+ Plasmacytoid dendritic cells (pDC), CD3/8/14/20-HLADR+ CD11c− CD123+ CD86+ Activated pDC, CD3/8/14/20− HLADR+ CD11c− CD123+ CD83+ Mature pDC, CD3/8/14/20− HLADR+ CD11c+ CD123− Myeloid dendritic cells (mDC), CD3/8/14/20− HLADR+ CD11c+ CD123− CD83+ Activated mDC.

Blood was drawn pre-dose and at 1, 2, 4, 8, 12, 16, 24, 48, 72, and 168 hr post-dose and processed to obtain serum. Serum cytokine levels were assessed at Boston University Analytical Instrumentation Core (Boston, MA) using a Monkey Magnetic 29-Plex Panel (ThermoFisher, Waltham, MA).

### MC38 Tumor Model

MC38 tumor studies were carried out at Crown Biosciences (Kannapolis, NC) according to Crown Biosciences approved IACUC protocols. MC38 tumor cells (1×10⁶ cells) were inoculated in the right flank of 7-8 week old female C57BL/6 mice. Treatment began once the average tumor volume reached 100 mm³ on approximately day 9 or 10. SNA was administered by intratumoral injection at the indicated dose level every 3 days for a total of 5 doses, except in the indicated studies where dosing was performed weekly for a total of 5 doses. Anti-PD-1 (Bio X Cell, West Lebanon, NH) was administered intraperitoneally at 5 mg/kg on the same days as SNA.

MC38 tumor cells (1×10⁶ cells) were inoculated for intraperitoneal challenge in naïve mice (n=6) or mice previously treated with SNA3 (1.6 mg/kg twice weekly)+anti-PD-1 (n=4) at 62 days following the initial tumor inoculation.

### EMT6 Tumor Model

EMT6 tumor studies were carried out at Oncodesign (Dijon, France) according to Oncodesign approved IACUC protocols. EMT6 tumor cells (1×10⁶ cells) were inoculated in the right flank of 6-7 week old female BALB/C mice. Treatment began three days after tumor inoculation at which time the average tumor volume was about 15 mm³ or when the average tumor volume reached 100 mm³ on day 10 after tumor inoculation. SNA was administered at the indicated dose level subcutaneously around the periphery of the tumor (peritumoral) every 3 days for a total of 5 doses. For combination studies, anti-PD-1 was administered intraperitoneally at 10 mg/kg every 5 days for a total of 3 doses beginning on day 5.

In experiments with intratumoral dosing, treatment began when the average tumor volume reached 100 mm³ on day 10 after tumor inoculation. SNA was administered by intratumoral injection at the indicated dose level every 7 days for a total of 5 doses.

In experiments with intravenous dosing, treatment began three days after tumor inoculation. SNA was administered by intravenous bolus injection into the caudal vein at 1-2 mg/kg as indicated every 3 days for a total of 5 doses.

For re-challenge experiments, mice previously treated with SNA3+ anti-PD-1 or naïve mice were inoculated in the flank with 1×10⁶ EMT6, CT26, or 4T1 tumor cells.

### Immunohistochemistry

Immunohistochemistry was performed at Biodoxis Laboratories (Romainville, France). FoxP3 staining was performed on 5 μm thick slices of formalin-fixed tumor samples. The number of FoxP3 positive cells per mm² of tumor was counted. CD8 staining was performed on cryopreserved tumor samples. CD8 infiltration was scored on a 0-4 scale, with zero indicating 0, one indicating 1-5, two indicating 6-10, three indicating 11-20, and four indicating >20 CD8 cells per 20× microscopy field.

### Flow Cytometry

Flow cytometry was performed at Oncodesign. Fresh, dissociated draining lymph node cells were stained with the following antibodies or isotype controls. T-cell panel: PD-1, FoxP3, CD4, IgG2b (Miltenyi Biotec, San Diego, CA), IgG2b, CD8a, IgG2a, CD25, IgG1, CD3, IgG2, CD45 (BD Biosciences, San Jose, CA), IgG1 (Beckman Coulter, Brea, CA). MDSC panel: CD274/PD-L1 (Acris/Interchim, Montluçon, France), IgG2a, CD3, IgG1, IgG2a, CD45, IgG2, CD11b, IgG2b (BD Biosciences), Ly-6G, REA Control S, Ly-6C, IgG2a, Inside Stain Kit (Miltenyi Biotec), iNOS/NOS2 (eBioscience, San Diego, CA), Arg1, IgG (R&D Systems, Minneapolis, MN). For each sample 10,000 CD45+ events were recorded using a CyFlow® Space flow cytometer. After gating on live leukocytes, each sub-population was displayed as percentage of the parental population.

### References

1. T. Tokunaga, H. Yamamoto, S. Shimada, H. Abe, T. Fukuda, Y. Fujisawa, Y. Furutani, O. Yano, T. Kataoka, T. Sudo, N. Makiguchi, T. Suganuma, Antitumor activity of deoxyribonucleic acid fraction from Mycobacterium bovis BCG. I. Isolation, physicochemical characterization, and antitumor activity. J Natl Cancer Inst 72, 955-962 (1984).
2. Y. Sato, M. Roman, H. Tighe, D. Lee, M. Corr, M. D. Nguyen, G. J. Silverman, M. Lotz, D. A. Carson, E. Raz, Immunostimulatory DNA sequences necessary for effective intradermal gene immunization. Science 273, 352-354 (1996).
3. A. M. Krieg, A. K. Yi, S. Matson, T. J. Waldschmidt, G. A. Bishop, R. Teasdale, G. A. Koretzky, D. M. Klinman, CpG motifs in bacterial DNA trigger direct B-cell activation. Nature 374, 546-549 (1995).
4. H. Hemmi, O. Takeuchi, T. Kawai, T. Kaisho, S. Sato, H. Sanjo, M. Matsumoto, K. Hoshino, H. Wagner, K. Takeda, S. Akira, A Toll-like receptor recognizes bacterial DNA. Nature 408, 740-745 (2000).
5. S. Bauer, C. J. Kirschning, H. Hacker, V. Redecke, S. Hausmann, S. Akira, H. Wagner, G. B. Lipford, Human TLR9 confers responsiveness to bacterial DNA via species-specific CpG motif recognition. Proc Natl Acad Sci U S A 98, 9237-9242 (2001).
6. D. M. Klinman, A. K. Yi, S. L. Beaucage, J. Conover, A. M. Krieg, CpG motifs present in bacteria DNA rapidly induce lymphocytes to secrete interleukin 6,interleukin 12, and interferon gamma. Proc Natl Acad Sci U S A 93, 2879-2883 (1996).
7. A. M. Krieg, Therapeutic potential of Toll-like receptor 9 activation. Nat Rev Drug Discov 5, 471-484 (2006).
8. J. Vollmer, R. Weeratna, P. Payette, M. Jurk, C. Schetter, M. Laucht, T. Wader, S. Tluk, M. Liu, H. L. Davis, A. M. Krieg, Characterization of three CpG oligodeoxynucleotide classes with distinct immunostimulatory activities. Eur J Immunol 34, 251-262 (2004).
9. D. Verthelyi, K. J. Ishii, M. Gursel, F. Takeshita, D. M. Klinman, Human peripheral blood cells differentially recognize and respond to two distinct CPG motifs. J Immunol 166, 2372-2377 (2001).
10. J. D. Marshall, K. Fearon, C. Abbate, S. Subramanian, P. Yee, J. Gregorio, R. L. Coffman, G. Van Nest, Identification of a novel CpG DNA class and motif that optimally stimulate B cell and plasmacytoid dendritic cell functions. J Leukoc Biol 73, 781-792 (2003).
11. J. Scheiermann, D. M. Klinman, Clinical evaluation of CpG oligonucleotides as adjuvants for vaccines targeting infectious diseases and cancer. Vaccine 32, 6377-6389 (2014).
12. Y. M. Murad, T. M. Clay, H. K. Lyerly, M. A. Morse, CPG-7909 (PF-3512676, ProMune): toll-like receptor-9 agonist in cancer therapy. Expert Opin Biol Ther 7, 1257-1266 (2007).
13. A. M. Krieg, CpG still rocks! Update on an accidental drug. Nucleic Acid Ther 22, 77-89 (2012).
14. C. A. Mirkin, R. L. Letsinger, R. C. Mucic, J. J. Storhoff, A DNA-based method for rationally assembling nanoparticles into macroscopic materials. Nature 382, 607-609 (1996).
15. J. I. Cutler, E. Auyeung, C. A. Mirkin, Spherical nucleic acids. J Am Chem Soc 134, 1376-1391 (2012).
16. S. N. Barnaby, T. L. Sita, S. H. Petrosko, A. H. Stegh, C. A. Mirkin, Therapeutic applications of spherical nucleic acids. Cancer Treat Res 166, 23-50 (2015).
17. A. F. Radovic-Moreno, N. Chernyak, C. C. Mader, S. Nallagatla, R. S. Kang, L. Hao, D. A. Walker, T. L. Halo, T. J. Merkel, C. H. Rische, S. Anantatmula, M. Burkhart, C. A. Mirkin, S. M. Gryaznov, Immunomodulatory spherical nucleic acids. Proc Natl Acad Sci U S A 112, 3892-3897 (2015).
18. P. C. Patel, D. A. Giljohann, D. S. Seferos, C. A. Mirkin, Peptide antisense nanoparticles. Proc Natl Acad Sci U S A 105, 17222-17226 (2008).
19. K. Zhang, L. Hao, S. J. Hurst, C. A. Mirkin, Antibody-linked spherical nucleic acids for cellular targeting. J Am Chem Soc 134, 16488-16491 (2012).
20. P. C. Patel, D. A. Giljohann, W. L. Daniel, D. Zheng, A. E. Prigodich, C. A. Mirkin, Scavenger receptors mediate cellular uptake of polyvalent oligonucleotide-functionalized gold nanoparticles. Bioconjug Chem 21, 2250-2256 (2010).
21. C. H. Choi, L. Hao, S. P. Narayan, E. Auyeung, C. A. Mirkin, Mechanism for the endocytosis of spherical nucleic acid nanoparticle conjugates. Proc Natl Acad Sci U S A 110, 7625-7630 (2013).
22. X. A. Wu, C. H. Choi, C. Zhang, L. Hao, C. A. Mirkin, Intracellular fate of spherical nucleic acid nanoparticle conjugates. J Am Chem Soc 136, 7726-7733 (2014).
23. S. W. Dow, L. G. Fradkin, D. H. Liggitt, A. P. Willson, T. D. Heath, T. A. Potter, Lipid-DNA complexes induce potent activation of innate immune responses and antitumor activity when administered intravenously. J Immunol 163, 1552-1561 (1999).
24. M. Whitmore, S. Li, L. Huang, LPD lipopolyplex initiates a potent cytokine response and inhibits tumor growth. Gene Ther 6, 1867-1875 (1999).
25. F. Sakurai, T. Terada, K. Yasuda, F. Yamashita, Y. Takakura, M. Hashida, The role of tissue macrophages in the induction of proinflammatory cytokine production following intravenous injection of lipoplexes. Gene Ther 9, 1120-1126 (2002).
26. D. Jarrossay, G. Napolitani, M. Colonna, F. Sallusto, A. Lanzavecchia, Specialization and complementarity in microbial molecule recognition by human myeloid and plasmacytoid dendritic cells. Eur J Immunol 31, 3388-3393 (2001).
27. V. Hornung, S. Rothenfusser, S. Britsch, A. Krug, B. Jahrsdorfer, T. Giese, S. Endres, G. Hartmann, Quantitative expression of toll-like receptor 1-10 mRNA in cellular subsets of human peripheral blood mononuclear cells and sensitivity to CpG oligodeoxynucleotides. J Immunol 168, 4531-4537 (2002).
28. A. D. Edwards, S. S. Diebold, E. M. Slack, H. Tomizawa, H. Hemmi, T. Kaisho, S. Akira, C. Reis e Sousa, Toll-like receptor expression in murine DC subsets: lack of TLR7 expression by CD8 alpha+DC correlates with unresponsiveness to imidazoquinolines. Eur J Immunol 33, 827-833 (2003).
29. M. Rehli, Of mice and men: species variations of Toll-like receptor expression. Trends Immunol 23, 375-378 (2002).
30. E. R. Kandimalla, L. Bhagat, Y. Li, D. Yu, D. Wang, Y. P. Cong, S. S. Song, J. X. Tang, T. Sullivan, S. Agrawal, Immunomodulatory oligonucleotides containing a cytosine-phosphate-2′-deoxy-7-deazaguanosine motif as potent toll-like receptor 9 agonists. Proc Natl Acad Sci U S A 102, 6925-6930 (2005).
31. A. M. Krieg, S. M. Efler, M. Wittpoth, M. J. Al Adhami, H. L. Davis, Induction of systemic TH1-like innate immunity in normal volunteers following subcutaneous but not intravenous administration of CPG 7909, a synthetic B-class CpG oligodeoxynucleotide TLR9 agonist. J Immunother 27, 460-471 (2004).
32. A. E. Vilgelm, D. B. Johnson, A. Richmond, Combinatorial approach to cancer immunotherapy: strength in numbers. J Leukoc Biol 100, 275-290 (2016).
33. S. L. Topalian, F. S. Hodi, J. R. Brahmer, S. N. Gettinger, D. C. Smith, D. F. McDermott, J. D. Powderly, R. D. Carvajal, J. A. Sosman, M. B. Atkins, P. D. Leming, D. R. Spigel, S. J. Antonia, L. Horn, C. G. Drake, D. M. Pardoll, L. Chen, W. H. Sharfman, R. A. Anders, J. M. Taube, T. L. McMiller, H. Xu, A. J. Korman, M. Jure-Kunkel, S. Agrawal, D. McDonald, G. D. Kollia, A. Gupta, J. M. Wigginton, M. Sznol, Safety, activity, and immune correlates of anti-PD-1 antibody in cancer. N Engl J Med 366, 2443-2454 (2012).
34. M. A. Postow, M. K. Callahan, J. D. Wolchok, Immune Checkpoint Blockade in Cancer Therapy. J Clin Oncol 33, 1974-1982 (2015).
35. M. J. Gray, J. Gong, M. M. Hatch, V. Nguyen, C. C. Hughes, J. T. Hutchins, B. D. Freimark, Phosphatidylserine-targeting antibodies augment the anti-tumorigenic activity of anti-PD-1 therapy by enhancing immune activation and downregulating pro-oncogenic factors induced by T-cell checkpoint inhibition in murine triple-negative breast cancers. Breast Cancer Res 18, 50 (2016).
36. J. W. Friedberg, J. L. Kelly, D. Neuberg, D. R. Peterson, J. L. Kutok, R. Salloum, T. Brenn, D. C. Fisher, E. Ronan, V. Dalton, L. Rich, D. Marquis, P. Sims, P. G. Rothberg, J. Liesveld, R. I. Fisher, R. Coffman, T. Mosmann, A. S. Freedman, Phase II study of a TLR-9 agonist (1018 ISS) with rituximab in patients with relapsed or refractory follicular lymphoma. Br J Haematol 146, 282-291 (2009).
37. E. Chan, E. L. Kwak, J. Hwang, M. Heiskala, G. de La Bourdonnaye, M. Mita, Open-label phase 1b study of FOLFIRI plus cetuximab plus IMO-2055 in patients with colorectal cancer who have progressed following chemotherapy for advanced or metastatic disease. Cancer Chemother Pharmacol 75, 701-709 (2015).
38. B. Wittig, M. Schmidt, W. Scheithauer, H. J. Schmoll, MGN1703, an immunomodulator and toll-like receptor 9 (TLR-9) agonist: from bench to bedside. Crit Rev Oncol Hematol 94, 31-44 (2015).
39. D. A. Giljohann, D. S. Seferos, A. E. Prigodich, P. C. Patel, C. A. Mirkin, Gene regulation with polyvalent siRNA-nanoparticle conjugates. J Am Chem Soc 131, 2072-2073 (2009).
40. D. M. Klinman, Use of CpG oligodeoxynucleotides as immunoprotective agents. Expert Opin Biol Ther 4, 937-946 (2004).
41. A. Krug, S. Rothenfusser, V. Hornung, B. Jahrsdorfer, S. Blackwell, Z. K. Ballas, S. Endres, A. M. Krieg, G. Hartmann, Identification of CpG oligonucleotide sequences with high induction of IFN-alpha/beta in plasmacytoid dendritic cells. Eur J Immunol 31, 2154-2163 (2001).
42. K. Abel, Y. Wang, L. Fritts, E. Sanchez, E. Chung, P. Fitzgerald-Bocarsly, A. M. Krieg, C. J. Miller, Deoxycytidyl-deoxyguanosine oligonucleotide classes A, B, and C induce distinct cytokine gene expression patterns in rhesus monkey peripheral blood mononuclear cells and distinct alpha interferon responses in TLR9-expressing rhesus monkey plasmacytoid dendritic cells. Clin Diagn Lab Immunol 12, 606-621 (2005).
43. M. M. Whitmore, A. Iparraguirre, L. Kubelka, W. Weninger, T. Hai, B. R. Williams, Negative regulation of TLR-signaling pathways by activating transcription factor-3. J Immunol 179, 3622-3630 (2007).
44. V. A. Stewart, S. McGrath, A. M. Krieg, N. S. Larson, E. Angov, C. L. Smith, T. G. Brewer, D. G. Heppner, Jr., Activation of innate immunity in healthy Macaca mulatta macaques by a single subcutaneous dose of GMP CpG 7909: safety data and interferon-inducible protein-10 kinetics for humans and macaques. Clin Vaccine Immunol 15, 221-226 (2008).
45. M. Kwissa, H. I. Nakaya, H. Oluoch, B. Pulendran, Distinct TLR adjuvants differentially stimulate systemic and local innate immune responses in nonhuman primates. Blood 119, 2044-2055 (2012).
46. W. E. Aulitzky, H. Tilg, W. Vogel, W. Aulitzky, M. Berger, G. Gastl, M. Herold, C. Huber, Acute hematologic effects of interferon alpha, interferon gamma, tumor necrosis factor alpha and interleukin 2. Ann Hematol 62, 25-31 (1991).
47. M. J. Robertson, D. Pelloso, R. Abonour, R. A. Hromas, R. P. Nelson, Jr., L. Wood, K. Cornetta, Interleukin 12 immunotherapy after autologous stem cell transplantation for hematological malignancies. Clin Cancer Res 8, 3383-3393 (2002).
48. S. Spranger, Mechanisms of tumor escape in the context of the T-cell-inflamed and the non-T-cell-inflamed tumor microenvironment. Int Immunol 28, 383-391 (2016).
49. A. A. Ashkar, K. L. Rosenthal, Toll-like receptor 9, CpG DNA and innate immunity. Curr Mol Med 2, 545-556 (2002).
50. Y. M. Murad, T. M. Clay, CpG oligodeoxynucleotides as TLR9 agonists: therapeutic applications in cancer. BioDrugs 23, 361-375 (2009).
51. C. Zoglmeier, H. Bauer, D. Norenberg, G. Wedekind, P. Bittner, N. Sandholzer, M. Rapp, D. Anz, S. Endres, C. Bourquin, CpG blocks immunosuppression by myeloid-derived suppressor cells in tumor-bearing mice. Clin Cancer Res 17, 1765-1775 (2011).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

What is claimed is:

1. An immunostimulatory spherical nucleic acid (IS-SNA), comprising a core having an oligonucleotide shell comprised of immunostimulatory oligonucleotides positioned on the exterior of the core.

2.-83. (canceled)

84. An immunostimulatory spherical nucleic acid (IS-SNA),
wherein the IS-SNA comprises a core and an oligonucleotide shell,
wherein the core is a liposomal core comprised of neutral lipids and the oligonucleotide shell is comprised of immunostimulatory oligonucleotides,
wherein the immunostimulatory oligonucleotides are CpG oligonucleotides comprising the sequence 5′-TCGTCGTTTTGTCGTTTTGTCGTT-3′ (SEQ ID NO: 5),
wherein the CpG oligonucleotides are each conjugated to a cholesterol via a linker comprising two hexaethylene glycols,
wherein the cholesterol anchors the CpG oligonucleotides to the lipids of the liposomal core,
wherein the immunostimulatory oligonucleotides are positioned on the exterior of the core such that the immunostimulatory oligonucleotides are oriented radially outwards, and
wherein each internucleoside linkage of the CpG oligonucleotides is a phosphorothioate internucleoside linkage.

85. The IS-SNA of claim 84, wherein the linker further comprises an additional oligoethylene glycol.

86. The IS-SNA of claim 84, wherein at least 25 immunostimulatory oligonucleotides are on the exterior of the core.

87. The IS-SNA of claim 84, wherein 25 to 50 immunostimulatory oligonucleotides are on the exterior of the core.

87. The IS-SNA of claim 84, wherein the core is comprised of one type of lipid, or wherein the core is comprised of 2-10 different lipids.

88. The IS-SNA of claim 84, wherein the neutral lipid is 1,2-dioleoyl-sn-glycero-3-phosphocholine (DOPC).

89. The IS-SNA of claim 84, wherein the core is about 20 nm in diameter.

90. A pharmaceutical composition, comprising the IS-SNA of claim 84.

91. A method for treating cancer comprising:
administering to a subject having cancer in an effective amount to treat the cancer an immunostimulatory spherical nucleic acid (IS-SNA) comprising a core and an oligonucleotide shell,
wherein the core is a liposomal core comprised of neutral lipids and the oligonucleotide shell is comprised of immunostimulatory oligonucleotides,
wherein the immunostimulatory oligonucleotides are CpG oligonucleotides comprising the sequence 5′-TCGTCGTTTTGTCGTTTTGTCGTT-3′ (SEQ ID NO: 5),
wherein the CpG oligonucleotides are each conjugated to a cholesterol via a linker comprising two hexaethylene glycols,
wherein the cholesterol anchors each CpG oligonucleotide to the lipids of the liposomal core,
wherein the immunostimulatory oligonucleotides are positioned on the exterior of the core such that the immunostimulatory oligonucleotides are oriented radially outwards, and
wherein each internucleoside linkage of the CpG oligonucleotides is an internucleoside phosphorothioate linkage.

92. The method of claim 91, wherein the linker further comprises an additional oligoethylene glycol.

93. The method of claim 91, wherein the IS-SNA is administered to the subject weekly for 4-12 weeks.

94. The method of claim 91, further comprising administering to the subject a checkpoint inhibitor.

95. The method of claim 91, wherein the immunostimulatory oligonucleotide in the IS-SNA increases the ratio of T-effector cells to T-regulatory cells in the subject relative to a linear immunostimulatory oligonucleotide not bound to an IS-SNA.

96. The method of claim 91, wherein the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma, bladder cell carcinoma, small cell lung cancer, non-small cell lung cancer, or colon carcinoma.

97. The method of claim 91, wherein the cancer is a skin cancer.

98. The method of claim 91, wherein the core is comprised of one type of neutral lipid, or wherein the core is comprised of 2-10 different neutral lipids.

99. The method of claim 91, wherein the neutral lipid is 1,2-dioleoyl-sn-glycero-3-phosphocholine (DOPC).

100. The method of claim 91, wherein the IS-SNA is administered via an intravenous injection, intratumoral injection or subcutaneous route of administration.

101. The method of claim 91, wherein the cancer is squamous cell carcinoma or liver cancer.
